SEQUENCE LISTING (76 sequences)

SEQ ID NO: 1 - protein, 131 aa, Homo sapiens
ARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKD

SEQ ID NO: 2 - protein, 118 aa, Homo sapiens
ACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKD

SEQ ID NO: 3 - protein, 132 aa, Cynocephalus sp.
AKSEDLYPNPKGSACSRIWQSPRFIARKRGFTVKMHCYVTNSTFSIVSWLRKRETDKEPQQVNLEQGHMHQTQNSSVTTLIIQDIRFEDNGIYFCQQECSKTSEVYRGCGTELRVMGFSTLAQLKQRNTLKD

SEQ ID NO: 4 - protein, 330 aa, Homo sapiens
ASTKGPSVFPLAPSSKSTSGGTAALGCLVKDYFPEPVTVSWSSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 107 aa, Homo sapiens [sequence omitted]

SEQ ID NO: 6 - protein, 18 aa, synthetic construct
MKWVTFISLLFLFSSAYS

SEQ ID NO: 7 - protein, 116 aa, synthetic construct
QSVEESGGRLVTPGTPLTLTCTASGFTINDYHMNWVRQAPGKGLEWIGIISSNGDTYYASWAKGRFTISRTSTTVDLKMTSLTTEDTATYFCARDDRDRSYAFDPWGPGTLVTVSS

SEQ ID NO: 8 - protein, 110 aa, synthetic construct
ALVMTQTPSSVSAAVGGTVTINCRASEDIERYLAWYQQKPGQRPKLLIYGASTLASGVPSRFKGSGSGTEYTLTISGVQPDDAATYYCHQGDSSRNVVNVFGGGTEVVVK

SEQ ID NO: 9 - protein, 5 aa, synthetic construct
DYHMN

SEQ ID NO: 10 - protein, 7 aa, synthetic construct
GFTINDY

SEQ ID NO: 11 - protein, 16 aa, synthetic construct
IISSNGDTYYASWAKG

SEQ ID NO: 12 - protein, 5 aa, synthetic construct
SSNGD

SEQ ID NO: 13 - protein, 11 aa, synthetic construct
DDRDRSYAFDP

SEQ ID NO: 14 - protein, 11 aa, synthetic construct
RASEDIERYLA

SEQ ID NO: 15 - protein, 7 aa, synthetic construct
GASTLAS

SEQ ID NO: 16 - protein, 12 aa, synthetic construct
HQGDSSRNVVNV

SEQ ID NO: 17 - protein, 115 aa, synthetic construct
QSLEESGGGLVTPGGTLTLTCTVSGLSLSSNAINWVRQAPGKGLEWIGMIYSGGRIEYASWAKGRFTISKTSTTVDLKITSPTTEDTATYFCAKGMSPSAHSSDIWGPGTLVTVS

SEQ ID NO: 18 - protein, 110 aa, synthetic construct
AYDMTQTPASVEVAVGGTVTIKCQASQSISDNLSWYQQKPGQPPKLLIYDASDLASGVPSRFSGSGSGTEFTLTISDLEPADAATYYCQQDWISSNVDNTFGGGTEVVVK

SEQ ID NO: 19 - protein, 5 aa, synthetic construct
SNAIN

SEQ ID NO: 20 - protein, 7 aa, synthetic construct
GLSLSSN

SEQ ID NO: 21 - protein, 16 aa, synthetic construct
MIYSGGRIEYASWAKG

SEQ ID NO: 22 - protein, 5 aa, synthetic construct
YSGGR

SEQ ID NO: 23 - protein, 11 aa, synthetic construct
GMSPSAHSSDI

SEQ ID NO: 24 - protein, 11 aa, synthetic construct
QASQSISDNLS

SEQ ID NO: 25 - protein, 7 aa, synthetic construct
DASDLAS

SEQ ID NO: 26 - protein, 12 aa, synthetic construct
QQDWISSNVDNT

SEQ ID NO: 27 - protein, 116 aa, synthetic construct
QSVKESGGGLFKPTDTLTLTCTVSGIDLNNYAMNWVRQAPGKGLDWIGLIGASGSTYYADWAKGRFTISKTSTTVDLKMTSLTTEDTATYFCAIGISATAHTSNIWGPGTLVAVSL

SEQ ID NO: 28 - protein, 110 aa, synthetic construct
ALVMTQTPSSVSAAVGGTVTINCQASQNIDSNLAWYQQKPGQPPKLLIYDASALASGVPSRFKGSGSGTEYTLTISGVEPDDAATYYCQQDDSSSNVDNPFGGGTEVVVK

SEQ ID NO: 29 - protein, 5 aa, synthetic construct
NYAMN

SEQ ID NO: 30 - protein, 7 aa, synthetic construct
GIDLNNY

SEQ ID NO: 31 - protein, 16 aa, synthetic construct
LIGASGSTYYADWAKG

SEQ ID NO: 32 - protein, 5 aa, synthetic construct
GASGS

SEQ ID NO: 33 - protein, 11 aa, synthetic construct
GISATAHTSNI

SEQ ID NO: 34 - protein, 11 aa, synthetic construct
QASQNIDSNLA

SEQ ID NO: 35 - protein, 7 aa, synthetic construct
DASALAS

SEQ ID NO: 36 - protein, 12 aa, synthetic construct
QQDDSSSNVDNP

SEQ ID NO: 37 - protein, 120 aa, synthetic construct
QSVRETGGGLVQPGGSLKLSCKASGFTINIDYWISWVRQAPGKGLEWIGIIDPGDGTTYFAIWVSGRFTISFDNTQNTVDLQMNSLTAADTATYFCVREFSASAYQYDLWGPGTLVTVSS

SEQ ID NO: 38 - protein, 110 aa, synthetic construct
DLVMTQTPSSVSAAVGGTVSINCRASEDIENYLAWYQQKPGQPPKLLIYRASTLSSGVSSRFSGSGSGTEFTLTISGVQPDDAATYYCQQGYRNSNVDNPFGGGTEMVVK

SEQ ID NO: 39 - protein, 6 aa, synthetic construct
IDYWIS

SEQ ID NO: 40 - protein, 8 aa, synthetic construct
GFTINIDY

SEQ ID NO: 41 - protein, 17 aa, synthetic construct
IIDPGDGTTYFAIWVSG

SEQ ID NO: 42 - protein, 6 aa, synthetic construct
DPGDGT

SEQ ID NO: 43 - protein, 11 aa, synthetic construct
EFSASAYQYDL

SEQ ID NO: 44 - protein, 11 aa, synthetic construct
RASEDIENYLA

SEQ ID NO: 45 - protein, 7 aa, synthetic construct
RASTLSS

SEQ ID NO: 46 - protein, 12 aa, synthetic construct
QQGYRNSNVDNP

SEQ ID NO: 47 - protein, 120 aa, synthetic construct
QSVKETGGGLVQPGGSLTLSCKASGFTINIDYWISWVRQAPGKGLEWIGIIDPGDGTTYFAIWVSGRFTISTDNAQNTVDLQMNSLTAADTATYFCAREFSASAYQYDLWGPGTLVTVSS

SEQ ID NO: 48 - protein, 110 aa, synthetic construct
ALVMTQTPSSVSAAVGGTVSINCQASEDIESYLTWYQQKPGQPPKLLMYRASTLSSGVSSRFSASGSGTEFTLTISGVQPDDAATYYCQQGYRNKDVDNPFGGGTEVVVK

SEQ ID NO: 49 - protein, 6 aa, synthetic construct
IDYWIS

SEQ ID NO: 50 - protein, 8 aa, synthetic construct
GFTINIDY

SEQ ID NO: 51 - protein, 17 aa, synthetic construct
IIDPGDGTTYFAIWVSG

SEQ ID NO: 52 - protein, 6 aa, synthetic construct
DPGDGT

SEQ ID NO: 53 - protein, 11 aa, synthetic construct
EFSASAYQYDL

SEQ ID NO: 54 - protein, 11 aa, synthetic construct
QASEDIESYLT

SEQ ID NO: 55 - protein, 7 aa, synthetic construct
RASTLSS

SEQ ID NO: 56 - protein, 12 aa, synthetic construct
QQGYRNKDVDNP

SEQ ID NO: 57 - protein, 120 aa, synthetic construct
QSVRETGGGLVQPGGSLTLSCEASEFTINIDYWISWVRQAPGKGLEWIGIIDPGDGTTYFAIWVSGRFTITRNTNLNTVDLKMTSLTEADTATYFCAREVSASAYQYDLWGPGTLVTVSS

SEQ ID NO: 58 - protein, 110 aa, synthetic construct
ALVMTQTPSSVSADVGGTVSINCQASEDIESYLAWYQQKPGQPPKLLIYFTSNLASGVPSRFKGSGSGTEYTLTISGVQPDDAATYYCQQGYRNSDVHNPFGGGTEVVVK

SEQ ID NO: 59 - protein, 6 aa, synthetic construct
IDYWIS

SEQ ID NO: 60 - protein, 8 aa, synthetic construct
EFTINIDY

SEQ ID NO: 61 - protein, 17 aa, synthetic construct
IIDPGDGTTYFAIWVSG

SEQ ID NO: 62 - protein, 6 aa, synthetic construct
DPGDGT

SEQ ID NO: 63 - protein, 11 aa, synthetic construct
EVSASAYQYDL

SEQ ID NO: 64 - protein, 11 aa, synthetic construct
QASEDIESYLA

SEQ ID NO: 65 - protein, 7 aa, synthetic construct
FTSNLAS

SEQ ID NO: 66 - protein, 12 aa, synthetic construct
QQGYRNSDVHNP

SEQ ID NO: 67 - protein, 125 aa, synthetic construct
MARLALSPVPSHWMVALLLLLSAEPVPAARSEDRYRNPKGFSTLAQLKQRNTLKDGIIMIQTLLIILFIIVPIFLLLDKDDSKAGMEEDHTYEGLDIDQTATYEDIVTLRTGEVKWSVGEHPGQE

SEQ ID NO: 68 - protein, 229 aa, synthetic construct
MARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLDKDDSKAGMEEDHTYEGLDIDQTATYEDIVTLRTGEVKWSVGEHPGQE

SEQ ID NO: 69 - protein, 119 aa, synthetic construct
EQQLLESGGGLVQPGGSLRLSCAVSGLSLSSNAINWVRQAPGKGLEWIGMIYSGGRIEYASWAKGRFTISKDSSKNTVYLQMNSLRAEDTAVYFCAKGMSPSAHSSDIWGPGTLVTVSS

SEQ ID NO: 70 - protein, 110 aa, synthetic construct
EGVMTQSPATLSVSPGERATLSCQASQSISDNLSWYQQKPGQPPRLLIYDASDLASGVPARFSGSGSGTEFTLTISSLQSEDFAVYYCQQDWISSNVDNTFGGGTKVEIK

SEQ ID NO: 71 - protein, 121 aa, synthetic construct
EQQLVETGGGLIQPGGSLRLSCAASGFTINIDYWISWVRQAPGKGLEWIGIIDPGDGTTYFAIWVSGRFTISFDNSKNTVYLQMNSLRAEDTAVYFCVREFSASAYQYDLWGPGTLVTVSS

SEQ ID NO: 72 - protein, 110 aa, synthetic construct
ELVMTQSPATLSVSPGERATLSCRASEDIENYLAWYQQKPGQPPRLLIYRASTLSSGVPARFSGSGSGTEFTLTISSLQSEDFAVYYCQQGYRNSNVDNPFGGGTKVEIK

SEQ ID NO: 73 - protein, 121 aa, synthetic construct
EQQLVETGGGLIQPGGSLRLSCAASGFTINIDYWISWVRQAPGKGLEWIGIIDPGDGTTYFAIWVSGRFTISTDNSKNTVYLQMNSLRAEDTAVYFCAREFSASAYQYDLWGPGTLVTVSS

SEQ ID NO: 74 - protein, 110 aa, synthetic construct
ELVMTQSPATLSVSPGERATLSCQASEDIESYLTWYQQKPGQPPRLLMYRASTLSSGVPARFSASGSGTEFTLTISSLQSEDFAVYYCQQGYRNKDVDNPFGGGTKVEIK

SEQ ID NO: 75 - protein, 121 aa, synthetic construct
EQQLVETGGGLIQPGGSLRLSCAASEFTINIDYWISWVRQAPGKGLEWIGIIDPGDGTTYFAIWVSGRFTISRDTSKNTVYLQMNSLRAEDTAVYFCAREVSASAYQYDLWGPGTLVTVSS

SEQ ID NO: 76 - protein, 110 aa, synthetic construct
ELVMTQSPATLSVSPGERATLSCQASEDIESYLAWYQQKPGQPPRLLIYFTSNLASGVPARFSGSGSGTEYTLTISSLQSEDFAVYYCQQGYRNSDVHNPFGGGTKVEIK

(19) United States
(12) Patent Application Publication
(10) Pub. No.: US 2026/0209346 A1
(43) Pub. Date: Jul. 23, 2026

(54) Anti-CD79b Antibodies and Uses Thereof

(71) Applicant: LTZ Therapeutics Inc., Redwood City, CA (US)

(72) Inventors: Jianhui Zhou, Redwood City, CA (US); Jinze Li, Redwood City, CA (US); Zhentao Sheng, Redwood City, CA (US); Jie Hu, Redwood City, CA (US); Martin Treder, Redwood City, CA (US); Su Huang, Redwood City, CA (US); Vivekananda Kedage, Redwood City, CA (US)

(21) Appl. No.: 19/142,497

(22) PCT Filed: Dec. 22, 2023
(86) PCT No.: PCT/US2023/085696

§ 371 Date: Jun. 23, 2025

Related U.S. Application Data
(60) Provisional application No. 63/435,896, filed on Dec. 29, 2022.

(51) Int. Cl. C07K 16/28; A61K 47/68; A61P 35/02; C07K 14/725

(52) U.S. Cl. CPC C07K 16/2803; A61K 47/6849; A61P 35/02; C07K 14/7051; C07K 2317/24; C07K 2317/31; C07K 2317/622

(57) ABSTRACT

This disclosure relates to anti-CD79b (Cluster of Differentiation 79B) antibodies, antigen-binding fragments thereof, antibody-drug conjugate (ADC) derived therefrom, and the uses thereof.

### CROSS-REFERENCE TO RELATED APPLICATIONS

This application claims the benefit of U.S. Provisional Application Ser. No. 63/435,896, filed Dec. 29, 2022. The disclosure of the prior application is considered part of the disclosure of this application, and is incorporated in its entirety into this application.

### SEQUENCE LISTING

This application contains a Sequence Listing that has been submitted electronically as an XML file named “55679-0002WO1 Sequence list.XML.” The XML file, created on Dec. 6, 2023, is 69,659 bytes in size. The material in the XML file is hereby incorporated by reference in its entirety.

### TECHNICAL FIELD

This disclosure relates to anti-CD79b (Cluster of Differentiation 79B) antibodies, antigen-binding fragments thereof, antibody-drug conjugate (ADC) derived therefrom, and the uses thereof.

### BACKGROUND

CD79 is the signaling component of the B-cell receptor and acts as a covalent heterodimer containing CD79a and CD79b. CD79b contains an extracellular immunoglobulin (Ig) domain, a transmembrane domain, and an intracellular signaling domain, an immunoreceptor tyrosine-based activation motif (ITAM) domain. CD79b expression has been detected on the surface of almost all non-Hodgkin lymphoma (NHL), acute lymphoblastic leukemia (ALL) and chronic lymphocytic leukemia (CLL) patients.

CD79b is an attractive therapeutic target for B-cell malignancies for various reasons. Firstly, not only the broad expression in B-cell malignancies, but also the unaltered expression after the loss of CD19 or CD20 make this receptor an attractive alternative for targeted treatment beyond CD19 or CD20 targeted treatment. Secondly, when the B-cell receptor is cross-linked, it is targeted to the major histocompatibility complex class II compartment, a lysosome-like compartment, as part of class II antigen presentation by B cells.

Considering the important role of CD79b in cancer, there is a need to develop therapeutic agents targeting CD79b.

### SUMMARY

This disclosure relates to anti-CD79b antibodies, antigen-binding fragment thereof, and the uses thereof.

In one aspect, the disclosure is related to an antibody or antigen-binding fragment thereof that binds to CD79b (Cluster of Differentiation 79B) comprising:
a heavy chain variable region (VH) comprising complementarity determining regions (CDRs) 1, 2, and 3, wherein the VH CDR1 region comprises an amino acid sequence that is at least 80% identical to a selected VH CDR1 amino acid sequence, the VH CDR2 region comprises an amino acid sequence that is at least 80% identical to a selected VH CDR2 amino acid sequence, and the VH CDR3 region comprises an amino acid sequence that is at least 80% identical to a selected VH CDR3 amino acid sequence; and
a light chain variable region (VL) comprising CDRs 1, 2, and 3, wherein the VL CDR1 region comprises an amino acid sequence that is at least 80% identical to a selected VL CDR1 amino acid sequence, the VL CDR2 region comprises an amino acid sequence that is at least 80% identical to a selected VL CDR2 amino acid sequence, and the VL CDR3 region comprises an amino acid sequence that is at least 80% identical to a selected VL CDR3 amino acid sequence,
wherein the selected VH CDRs 1, 2, and 3 amino acid sequences and the selected VL CDRs, 1, 2, and 3 amino acid sequences are one of the following:
(1) the selected VH CDRs 1, 2, 3 amino acid sequences are set forth in SEQ ID NOs: 9, 11, 13, respectively, and the selected VL CDRs 1, 2, 3 amino acid sequences are set forth in SEQ ID NOs: 14-16, respectively;
(2) the selected VH CDRs 1, 2, 3 amino acid sequences are set forth in SEQ ID NOs: 19, 21, 23, respectively, and the selected VL CDRs 1, 2, 3 amino acid sequences are set forth in SEQ ID NOs: 24-26, respectively;
(3) the selected VH CDRs 1, 2, 3 amino acid sequences are set forth in SEQ ID NOs: 29, 31, 33, respectively, and the selected VL CDRs 1, 2, 3 amino acid sequences are set forth in SEQ ID NOs: 34-36, respectively;
(4) the selected VH CDRs 1, 2, 3 amino acid sequences are set forth in SEQ ID NOs: 39, 41, 43, respectively, and the selected VL CDRs 1, 2, 3 amino acid sequences are set forth in SEQ ID NOs: 44-46, respectively;
(5) the selected VH CDRs 1, 2, 3 amino acid sequences are set forth in SEQ ID NOs: 49, 51, 53, respectively, and the selected VL CDRs 1, 2, 3 amino acid sequences are set forth in SEQ ID NOs: 54-56, respectively;
(6) the selected VH CDRs 1, 2, 3 amino acid sequences are set forth in SEQ ID NOs: 59, 61, 63, respectively, and the selected VL CDRs 1, 2, 3 amino acid sequences are set forth in SEQ ID NOs: 64-66, respectively;
(7) the selected VH CDRs 1, 2, 3 amino acid sequences are set forth in SEQ ID NOs: 10, 12, 13, respectively, and the selected VL CDRs 1, 2, 3 amino acid sequences are set forth in SEQ ID NOs: 14-16, respectively;
(8) the selected VH CDRs 1, 2, 3 amino acid sequences are set forth in SEQ ID NOs: 20, 22, 23, respectively, and the selected VL CDRs 1, 2, 3 amino acid sequences are set forth in SEQ ID NOs: 24-26, respectively;
(9) the selected VH CDRs 1, 2, 3 amino acid sequences are set forth in SEQ ID NOs: 30, 32, 33, respectively, and the selected VL CDRs 1, 2, 3 amino acid sequences are set forth in SEQ ID NOs: 34-36, respectively;
(10) the selected VH CDRs 1, 2, 3 amino acid sequences are set forth in SEQ ID NOs: 40, 42, 43, respectively, and the selected VL CDRs 1, 2, 3 amino acid sequences are set forth in SEQ ID NOs: 44-46, respectively;
(11) the selected VH CDRs 1, 2, 3 amino acid sequences are set forth in SEQ ID NOs: 50, 52, 53, respectively, and the selected VL CDRs 1, 2, 3 amino acid sequences are set forth in SEQ ID NOs: 54-56, respectively; and
(12) the selected VH CDRs 1, 2, 3 amino acid sequences are set forth in SEQ ID NOs: 60, 62, 63, respectively, and the selected VL CDRs 1, 2, 3 amino acid sequences are set forth in SEQ ID NOs: 64-66, respectively.

In some embodiments, the VH comprises CDRs 1, 2, 3 with the amino acid sequences set forth in SEQ ID NOs: 9, 11, 13, respectively, and the VL comprises CDRs 1, 2, 3 with the amino acid sequences set forth in SEQ ID NOs: 14-16, respectively, according to Kabat definition.

In some embodiments, the VH comprises CDRs 1, 2, 3 with the amino acid sequences set forth in SEQ ID NOs: 19, 21, 23, respectively, and the VL comprises CDRs 1, 2, 3 with the amino acid sequences set forth in SEQ ID NOs: 24-26, respectively, according to Kabat definition.

In some embodiments, the VH comprises CDRs 1, 2, 3 with the amino acid sequences set forth in SEQ ID NOs: 29, 31, 33, respectively, and the VL comprises CDRs 1, 2, 3 with the amino acid sequences set forth in SEQ ID NOs: 34-36, respectively, according to Kabat definition.

In some embodiments, the VH comprises CDRs 1, 2, 3 with the amino acid sequences set forth in SEQ ID NOs: 39, 41, 43, respectively, and the VL comprises CDRs 1, 2, 3 with the amino acid sequences set forth in SEQ ID NOs: 44-46, respectively, according to Kabat definition.

In some embodiments, the VH comprises CDRs 1, 2, 3 with the amino acid sequences set forth in SEQ ID NOs: 49, 51, 53, respectively, and the VL comprises CDRs 1, 2, 3 with the amino acid sequences set forth in SEQ ID NOs: 54-56, respectively, according to Kabat definition.

In some embodiments, the VH comprises CDRs 1, 2, 3 with the amino acid sequences set forth in SEQ ID NOs: 59, 61, 63, respectively, and the VL comprises CDRs 1, 2, 3 with the amino acid sequences set forth in SEQ ID NOs: 64-66, respectively, according to Kabat definition.

In some embodiments, the VH comprises CDRs 1, 2, 3 with the amino acid sequences set forth in SEQ ID NOs: 10, 12, 13, respectively, and the VL comprises CDRs 1, 2, 3 with the amino acid sequences set forth in SEQ ID NOs: 14-16, respectively, according to Chothia definition.

In some embodiments, the VH comprises CDRs 1, 2, 3 with the amino acid sequences set forth in SEQ ID NOs: 20, 22, 23, respectively, and the VL comprises CDRs 1, 2, 3 with the amino acid sequences set forth in SEQ ID NOs: 24-26, respectively, according to Chothia definition.

In some embodiments, the VH comprises CDRs 1, 2, 3 with the amino acid sequences set forth in SEQ ID NOs: 30, 32, 33, respectively, and the VL comprises CDRs 1, 2, 3 with the amino acid sequences set forth in SEQ ID NOs: 34-36, respectively, according to Chothia definition.

In some embodiments, the VH comprises CDRs 1, 2, 3 with the amino acid sequences set forth in SEQ ID NOs: 40, 42, 43, respectively, and the VL comprises CDRs 1, 2, 3 with the amino acid sequences set forth in SEQ ID NOs: 44-46, respectively, according to Chothia definition.

In some embodiments, the VH comprises CDRs 1, 2, 3 with the amino acid sequences set forth in SEQ ID NOs: 50, 52, 53, respectively, and the VL comprises CDRs 1, 2, 3 with the amino acid sequences set forth in SEQ ID NOs: 54-56, respectively, according to Chothia definition.

In some embodiments, the VH comprises CDRs 1, 2, 3 with the amino acid sequences set forth in SEQ ID NOs: 60, 62, 63, respectively, and the VL comprises CDRs 1, 2, 3 with the amino acid sequences set forth in SEQ ID NOs: 64-66, respectively, according to Chothia definition.

In some embodiments, the antibody or antigen-binding fragment thereof specifically binds to human, mouse, monkey, or dog CD79b.

In some embodiments, the antibody or antigen-binding fragment thereof is a humanized antibody or antigen-binding fragment thereof, a single-chain variable fragment (scFv), a one-armed antibody, and/or a multi-specific antibody (e.g., a bispecific antibody).

In some embodiments, the antibody or antigen-binding fragment thereof comprises a human IgG1 constant region, a human IgG2 constant region, or a human IgG4 constant region.

In one aspect, the disclosure is related to a nucleic acid comprising a polynucleotide encoding a polypeptide comprising:
(1) an immunoglobulin heavy chain or a fragment thereof comprising a VH comprising complementarity determining regions (CDRs) 1, 2, and 3 comprising the amino acid sequences set forth in SEQ ID NOs: 9, 11, 13, respectively, and wherein the VH, when paired with a light chain variable region (VL) comprising the amino acid sequence set forth in SEQ ID NO: 8 binds to CD79b;
(2) an immunoglobulin light chain or a fragment thereof comprising a VL comprising complementarity determining regions (CDRs) 1, 2, and 3 comprising the amino acid sequences set forth in SEQ ID NOs: 14-16, respectively, and wherein the VL, when paired with a VH comprising the amino acid sequence set forth in SEQ ID NO: 7 binds to CD79b;
(3) an immunoglobulin heavy chain or a fragment thereof comprising a VH comprising complementarity determining regions (CDRs) 1, 2, and 3 comprising the amino acid sequences set forth in SEQ ID NOs: 19, 21, 23, respectively, and wherein the VH, when paired with a light chain variable region (VL) comprising the amino acid sequence set forth in SEQ ID NO: 18 binds to CD79b;
(4) an immunoglobulin light chain or a fragment thereof comprising a VL comprising complementarity determining regions (CDRs) 1, 2, and 3 comprising the amino acid sequences set forth in SEQ ID NOs: 24-26, respectively, and wherein the VL, when paired with a VH comprising the amino acid sequence set forth in SEQ ID NO: 17 binds to CD79b;
(5) an immunoglobulin heavy chain or a fragment thereof comprising a VH comprising complementarity determining regions (CDRs) 1, 2, and 3 comprising the amino acid sequences set forth in SEQ ID NOs: 29, 31, 33, respectively, and wherein the VH, when paired with a light chain variable region (VL) comprising the amino acid sequence set forth in SEQ ID NO: 28 binds to CD79b;
(6) an immunoglobulin light chain or a fragment thereof comprising a VL comprising CDRs 1, 2, and 3 comprising the amino acid sequences set forth in SEQ ID NOs: 34-36, respectively, and wherein the VL, when paired with a VH comprising the amino acid sequence set forth in SEQ ID NO: 27 binds to CD79b,
(7) an immunoglobulin heavy chain or a fragment thereof comprising a VH comprising complementarity determining regions (CDRs) 1, 2, and 3 comprising the amino acid sequences set forth in SEQ ID NOs: 39, 41, 43, respectively, and wherein the VH, when paired with a light chain variable region (VL) comprising the amino acid sequence set forth in SEQ ID NO: 38 binds to CD79b;
(8) an immunoglobulin light chain or a fragment thereof comprising a VL comprising CDRs 1, 2, and 3 comprising the amino acid sequences set forth in SEQ ID NOs: 44-46, respectively, and wherein the VL, when paired with a VH comprising the amino acid sequence set forth in SEQ ID NO: 37 binds to CD79b;
(9) an immunoglobulin heavy chain or a fragment thereof comprising a VH comprising complementarity determining regions (CDRs) 1, 2, and 3 comprising the amino acid sequences set forth in SEQ ID NOs: 49, 51, 53, respectively, and wherein the VH, when paired with a light chain variable region (VL) comprising the amino acid sequence set forth in SEQ ID NO: 48 binds to CD79b;
(10) an immunoglobulin light chain or a fragment thereof comprising a VL comprising CDRs 1, 2, and 3 comprising the amino acid sequences set forth in SEQ ID NOs: 54-56, respectively, and wherein the VL, when paired with a VH comprising the amino acid sequence set forth in SEQ ID NO: 47 binds to CD79b;
(11) an immunoglobulin heavy chain or a fragment thereof comprising a VH comprising complementarity determining regions (CDRs) 1, 2, and 3 comprising the amino acid sequences set forth in SEQ ID NOs: 59, 61, 63, respectively, and wherein the VH, when paired with a light chain variable region (VL) comprising the amino acid sequence set forth in SEQ ID NO: 58 binds to CD79b;
(12) an immunoglobulin light chain or a fragment thereof comprising a VL comprising CDRs 1, 2, and 3 comprising the amino acid sequences set forth in SEQ ID NOs: 64-66, respectively, and wherein the VL, when paired with a VH comprising the amino acid sequence set forth in SEQ ID NO: 57 binds to CD79b;
(13) an immunoglobulin heavy chain or a fragment thereof comprising a VH comprising complementarity determining regions (CDRs) 1, 2, and 3 comprising the amino acid sequences set forth in SEQ ID NOs: 10, 12, 13, respectively, and wherein the VH, when paired with a light chain variable region (VL) comprising the amino acid sequence set forth in SEQ ID NO: 8 binds to CD79b;
(14) an immunoglobulin heavy chain or a fragment thereof comprising a VH comprising complementarity determining regions (CDRs) 1, 2, and 3 comprising the amino acid sequences set forth in SEQ ID NOs: 20, 22, 23, respectively, and wherein the VH, when paired with a light chain variable region (VL) comprising the amino acid sequence set forth in SEQ ID NO: 18 binds to CD79b;
(15) an immunoglobulin heavy chain or a fragment thereof comprising a VH comprising complementarity determining regions (CDRs) 1, 2, and 3 comprising the amino acid sequences set forth in SEQ ID NOs: 30, 32, 33, respectively, and wherein the VH, when paired with a light chain variable region (VL) comprising the amino acid sequence set forth in SEQ ID NO: 28 binds to CD79b;
(16) an immunoglobulin heavy chain or a fragment thereof comprising a VH comprising complementarity determining regions (CDRs) 1, 2, and 3 comprising the amino acid sequences set forth in SEQ ID NOs: 40, 42, 43, respectively, and wherein the VH, when paired with a light chain variable region (VL) comprising the amino acid sequence set forth in SEQ ID NO: 38 binds to CD79b;
(17) an immunoglobulin heavy chain or a fragment thereof comprising a VH comprising complementarity determining regions (CDRs) 1, 2, and 3 comprising the amino acid sequences set forth in SEQ ID NOs: 50, 52, 53, respectively, and wherein the VH, when paired with a light chain variable region (VL) comprising the amino acid sequence set forth in SEQ ID NO: 48 binds to CD79b;
(18) an immunoglobulin heavy chain or a fragment thereof comprising a VH comprising complementarity determining regions (CDRs) 1, 2, and 3 comprising the amino acid sequences set forth in SEQ ID NOs: 60, 62, 63, respectively, and wherein the VH, when paired with a light chain variable region (VL) comprising the amino acid sequence set forth in SEQ ID NO: 58 binds to CD79b;
(19) an immunoglobulin heavy chain or a fragment thereof comprising a VH comprising complementarity determining regions (CDRs) 1, 2, and 3 comprising the amino acid sequences set forth in SEQ ID NOs: 19, 21, 23, respectively, and wherein the VH, when paired with a light chain variable region (VL) comprising the amino acid sequence set forth in SEQ ID NO: 70 binds to CD79b;
(20) an immunoglobulin light chain or a fragment thereof comprising a VL comprising complementarity determining regions (CDRs) 1, 2, and 3 comprising the amino acid sequences set forth in SEQ ID NOs: 24-26, respectively, and wherein the VL, when paired with a VH comprising the amino acid sequence set forth in SEQ ID NO: 69 binds to CD79b;
(21) an immunoglobulin heavy chain or a fragment thereof comprising a VH comprising complementarity determining regions (CDRs) 1, 2, and 3 comprising the amino acid sequences set forth in SEQ ID NOs: 20, 22, 23, respectively, and wherein the VH, when paired with a light chain variable region (VL) comprising the amino acid sequence set forth in SEQ ID NO: 70 binds to CD79b;
(22) an immunoglobulin heavy chain or a fragment thereof comprising a VH comprising complementarity determining regions (CDRs) 1, 2, and 3 comprising the amino acid sequences set forth in SEQ ID NOs: 39, 41, 43, respectively, and wherein the VH, when paired with a light chain variable region (VL) comprising the amino acid sequence set forth in SEQ ID NO: 72 binds to CD79b
(23) an immunoglobulin light chain or a fragment thereof comprising a VL comprising CDRs 1, 2, and 3 comprising the amino acid sequences set forth in SEQ ID NOs: 44-46, respectively, and wherein the VL, when paired with a VH comprising the amino acid sequence set forth in SEQ ID NO: 71 binds to CD79b
(24) an immunoglobulin heavy chain or a fragment thereof comprising a VH comprising complementarity determining regions (CDRs) 1, 2, and 3 comprising the amino acid sequences set forth in SEQ ID NOs: 40, 42, 43, respectively, and wherein the VH, when paired with a light chain variable region (VL) comprising the amino acid sequence set forth in SEQ ID NO: 72 binds to CD79b;
(25) an immunoglobulin heavy chain or a fragment thereof comprising a VH comprising complementarity determining regions (CDRs) 1, 2, and 3 comprising the amino acid sequences set forth in SEQ ID NOs: 49, 51, 53, respectively, and wherein the VH, when paired with a light chain variable region (VL) comprising the amino acid sequence set forth in SEQ ID NO: 73 binds to CD79b;
(26) an immunoglobulin light chain or a fragment thereof comprising a VL comprising CDRs 1, 2, and 3 comprising the amino acid sequences set forth in SEQ ID NOs: 54-56, respectively, and wherein the VL, when paired with a VH comprising the amino acid sequence set forth in SEQ ID NO: 74 binds to CD79b;
(27) an immunoglobulin heavy chain or a fragment thereof comprising a VH comprising complementarity determining regions (CDRs) 1, 2, and 3 comprising the amino acid sequences set forth in SEQ ID NOs: 50, 52, 53, respectively, and wherein the VH, when paired with a light chain variable region (VL) comprising the amino acid sequence set forth in SEQ ID NO: 73 binds to CD79b
(28) an immunoglobulin heavy chain or a fragment thereof comprising a VH comprising complementarity determining regions (CDRs) 1, 2, and 3 comprising the amino acid sequences set forth in SEQ ID NOs: 59, 61, 63, respectively, and wherein the VH, when paired with a light chain variable region (VL) comprising the amino acid sequence set forth in SEQ ID NO: 76 binds to CD79b;
(29) an immunoglobulin light chain or a fragment thereof comprising a VL comprising CDRs 1, 2, and 3 comprising the amino acid sequences set forth in SEQ ID NOs: 64-66, respectively, and wherein the VL, when paired with a VH comprising the amino acid sequence set forth in SEQ ID NO: 75 binds to CD79b;
(30) an immunoglobulin heavy chain or a fragment thereof comprising a VH comprising complementarity determining regions (CDRs) 1, 2, and 3 comprising the amino acid sequences set forth in SEQ ID NOs: 60, 62, 63, respectively, and wherein the VH, when paired with a light chain variable region (VL) comprising the amino acid sequence set forth in SEQ ID NO: 76 binds to CD79b.

In some embodiments, the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 14-16, respectively.

In some embodiments, the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 24-26, respectively.

In some embodiments, the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 34-36, respectively.

In some embodiments, the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 44-46, respectively.

In some embodiments, the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 54-56, respectively.

In some embodiments, the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 64-66, respectively.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 9, 11, 13, respectively.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 19, 21, 23, respectively.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 29, 31, 33, respectively.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 39, 41, 43, respectively.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 49, 51, 53, respectively.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 59, 61, 63, respectively.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 10, 12, 13, respectively.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 20, 22, 23, respectively.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 30, 32, 33, respectively.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 40, 42, 43, respectively.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 50, 52, 53, respectively.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 60, 62, 63, respectively.

In some embodiments, the VH when paired with a VL specifically binds to human, mouse, monkey, or dog CD79b, or the VL when paired with a VH specifically binds to human, mouse, monkey, or dog CD79b.

In some embodiments, the immunoglobulin heavy chain or the fragment thereof comprises a human immunoglobulin heavy chain fragment (e.g., a human IgG1 heavy chain CH1, CH2, and/or CH3, a human IgG2 heavy chain CH1, CH2, and/or CH3, or a human IgG4 heavy chain CH1, CH2, and/or CH3), and the immunoglobulin light chain or the fragment thereof comprises a human immunoglobulin light chain constant region.

In some embodiments, the nucleic acid encodes a single-chain variable fragment (scFv), a one-armed antibody, a multi-specific antibody (e.g., a bispecific antibody), or a chimeric antigen receptor (CAR).

In some embodiments, the nucleic acid is cDNA.

In one aspect, the disclosure is related to a vector comprising one or more of the nucleic acids described herein.

In one aspect, the disclosure is related to a vector comprising two of the nucleic acids described herein, wherein the vector encodes the VL region and the VH region that together bind to CD79b.

In one aspect, the disclosure is related to a pair of vectors, wherein each vector comprises one of the nucleic acids described herein, wherein together the pair of vectors encodes the VL region and the VH region that together bind to CD79b.

In one aspect, the disclosure is related to a cell comprising the vector described herein, or the pair of vectors described herein.

In some embodiments, the cell is a CHO cell.

In one aspect, the disclosure is related to a cell comprising one or more of the nucleic acids described herein.

In one aspect, the disclosure is related to a cell comprising two of the nucleic acids described herein.

In some embodiments, the two nucleic acids together encode the VL region and the VH region that together bind to CD79b.

In one aspect, the disclosure is related to a method of producing an antibody or an antigen-binding fragment thereof, the method comprising
(a) culturing the cell described herein under conditions sufficient for the cell to produce the antibody or the antigen-binding fragment; and
(b) collecting the antibody or the antigen-binding fragment produced by the cell.

In one aspect, the disclosure is related to an antibody or antigen-binding fragment thereof that binds to CD79b comprising
a heavy chain variable region (VH) comprising an amino acid sequence that is at least 90% identical to a selected VH sequence, and a light chain variable region (VL) comprising an amino acid sequence that is at least 90% identical to a selected VL sequence, wherein the selected VH sequence and the selected VL sequence are one of the following:
(1) the selected VH sequence is SEQ ID NO: 7, and the selected VL sequence is SEQ ID NO: 8;
(2) the selected VH sequence is SEQ ID NO: 17 or 69, and the selected VL sequence is SEQ ID NO: 18 or 70;
(3) the selected VH sequence is SEQ ID NO: 27, and the selected VL sequence is SEQ ID NO: 28;
(4) the selected VH sequence is SEQ ID NO: 37 or 71, and the selected VL sequence is SEQ ID NO: 38 or 72;
(5) the selected VH sequence is SEQ ID NO: 47 or 73, and the selected VL sequence is SEQ ID NO: 48 or 74;
(6) the selected VH sequence is SEQ ID NO: 57 or 75, and the selected VL sequence is SEQ ID NO: 58 or 76.

In some embodiments, the VH comprises the sequence of SEQ ID NO: 7 and the VL comprises the sequence of SEQ ID NO: 8.

In some embodiments, the VH comprises the sequence of SEQ ID NO: 17 and the VL comprises the sequence of SEQ ID NO: 18.

In some embodiments, the VH comprises the sequence of SEQ ID NO: 27 and the VL comprises the sequence of SEQ ID NO: 28.

In some embodiments, the VH comprises the sequence of SEQ ID NO: 37 and the VL comprises the sequence of SEQ ID NO: 38.

In some embodiments, the VH comprises the sequence of SEQ ID NO: 47 and the VL comprises the sequence of SEQ ID NO: 48.

In some embodiments, the VH comprises the sequence of SEQ ID NO: 57 and the VL comprises the sequence of SEQ ID NO: 58.

In some embodiments, the VH comprises the sequence of SEQ ID NO: 69 and the VL comprises the sequence of SEQ ID NO: 70.

In some embodiments, the VH comprises the sequence of SEQ ID NO: 71 and the VL comprises the sequence of SEQ ID NO: 72.

In some embodiments, the VH comprises the sequence of SEQ ID NO: 73 and the VL comprises the sequence of SEQ ID NO: 74.

In some embodiments, the VH comprises the sequence of SEQ ID NO: 75 and the VL comprises the sequence of SEQ ID NO: 76.

In one aspect, the disclosure is related to an antibody or antigen-binding fragment thereof that binds to CD79b comprising
a heavy chain variable region (VH) comprising VH CDR1, VH CDR2, and VH CDR3 that are identical to VH CDR1, VH CDR2, and VH CDR3 of a selected VH sequence; and a light chain variable region (VL) comprising VL CDR1, VL CDR2, and VL CDR3 that are identical to VL CDR1, VL CDR2, and VL CDR3 of a selected VL sequence, wherein the selected VH sequence and the selected VL sequence are one of the following:
(1) the selected VH sequence is SEQ ID NO: 7, and the selected VL sequence is SEQ ID NO: 8;
(2) the selected VH sequence is SEQ ID NO: 17 or 69, and the selected VL sequence is SEQ ID NO: 18 or 70;
(3) the selected VH sequence is SEQ ID NO: 27, and the selected VL sequence is SEQ ID NO: 28;
(4) the selected VH sequence is SEQ ID NO: 37 or 71, and the selected VL sequence is SEQ ID NO: 38 or 72;
(5) the selected VH sequence is SEQ ID NO: 47 or 73, and the selected VL sequence is SEQ ID NO: 48 or 74; and
(6) the selected VH sequence is SEQ ID NO: 57 or 75, and the selected VL sequence is SEQ ID NO: 58 or 76.

In some embodiments, the antibody or antigen-binding fragment thereof specifically binds to human, mouse, monkey, or dog CD79b.

In some embodiments, the antibody or antigen-binding fragment thereof is a humanized antibody or antigen-binding fragment thereof, a chimeric antibody, a single-chain variable fragment (scFv), a one-armed antibody, and/or a multi-specific antibody (e.g., a bispecific antibody).

In some embodiments, the antibody or antigen-binding fragment comprises a human IgG1 Fc, a human IgG2 Fc, or a human IgG4 Fc.

In one aspect, the disclosure is related to an antibody or antigen-binding fragment thereof that cross-competes with the antibody or antigen-binding fragment thereof described herein.

In some embodiments, the antibody or antigen-binding fragment thereof comprises a fragment crystallizable region (Fc region).

In one aspect, the disclosure is related to a chimeric antigen receptor (CAR) comprising the antibody or antigen-binding fragment thereof described herein.

In one aspect, the disclosure is related to an antibody-drug conjugate comprising the antibody or antigen-binding fragment thereof described herein covalently bound to a therapeutic agent.

In some embodiments, the therapeutic agent is a cytotoxic or cytostatic agent.

In one aspect, the disclosure is related to a method of treating a subject having cancer, the method comprising administering a therapeutically effective amount of a composition comprising the antibody or antigen-binding fragment thereof described herein, the CAR described herein, or the antibody-drug conjugate described herein, to the subject.

In some embodiments, the cancer is lymphoma, leukemia, breast cancer, stomach cancer, pancreatic cancer, prostate cancer, cervical cancer, endometrial cancer, ovarian cancer or urothelial cancer.

In some embodiments, the cancer is Non-Hodgkin lymphoma (NHL), Diffuse large B-cell lymphoma (DLBCL), B acute lymphoblastic leukemia (B-ALL), chronic lymphocytic leukemia (CLL), B-cell prolymphocytic leukemia (PLL), splenic lymphoma with villous lymphocytes (SLVL), hairy cell leukemia (HCL), follicular lymphoma (FL) or mantle-cell (MCL) lymphoma.

In some embodiments, the subject is further treated with an effective amount of an anti-4-1BB antibody, an anti-OX40 antibody, an anti-PD-1 antibody, an anti-CTLA4 antibody, or an anti-CD40 antibody.

In one aspect, the disclosure is related to a method of decreasing the rate of tumor growth, the method comprising contacting a tumor cell with an effective amount of a composition comprising an antibody or antigen-binding fragment thereof described herein, the CAR described herein, or the antibody-drug conjugate described herein.

In one aspect, the disclosure is related to a method of killing a tumor cell, the method comprising contacting a tumor cell with an effective amount of a composition comprising the antibody or antigen-binding fragment thereof described herein, the CAR described herein, or the antibody-drug conjugate described herein.

In one aspect, the disclosure is related to a method of increasing immune response in a subject, the method comprising administering to the subject an effective amount of a composition comprising the antibody or antigen-binding fragment thereof described herein, the CAR described herein, or the antibody-drug conjugate described herein.

In one aspect, the disclosure is related to a method of treating a subject having autoimmune disease, the method comprising administering a therapeutically effective amount of a composition comprising a composition comprising the antibody or antigen-binding fragment thereof described herein, the CAR described herein, or the antibody-drug conjugate described herein.

In some embodiments, the autoimmune disease is selected from rheumatoid arthritis, psoriasis, multiple sclerosis, immune thrombocytopenic purpura, myasthenia gravis, neuromyelitis optica, IgG4-related diseases, systemic Lupus Erythematosus, lupus nephritis, giant cell arteritis, takayasu disease, cold agglutinin disease, warm autoimmune hemolytic anemia, and anti-neutrophil cytoplasmic antibody (ANCA) associated vasculitides, tranulomatosis with polyangiitis (GPA) (Wegener's Granulomatosis), Microscopic Polyangiitis (MPA), inflammatory bowel disease (IBD) or autoreactive pancreatitis.

In some embodiments, the autoimmune disease is multiple sclerosis, systemic lupus erythematosus, rheumatoid arthritis, inflammatory bowel disease (IBD) or autoreactive pancreatitis.

In one aspect, the disclosure is related to a pharmaceutical composition comprising the antibody or antigen-binding fragment thereof described herein, and a pharmaceutically acceptable carrier.

In one aspect, the disclosure is related to a pharmaceutical composition comprising the antibody drug conjugate described herein, and a pharmaceutically acceptable carrier.

As used herein, the term “cancer” refers to cells having the capacity for autonomous growth. Examples of such cells include cells having an abnormal state or condition characterized by rapidly proliferating cell growth. The term is meant to include cancerous growths, e.g., tumors; oncogenic processes, metastatic tissues, and malignantly transformed cells, tissues, or organs, irrespective of histopathologic type or stage of invasiveness. Also included are malignancies of the various organ systems, such as respiratory, cardiovascular, renal, reproductive, hematological, neurological, hepatic, gastrointestinal, and endocrine systems; as well as adenocarcinomas which include malignancies such as most colon cancers, renal-cell carcinoma, prostate cancer and/or testicular tumors, non-small cell carcinoma of the lung, and cancer of the small intestine. Cancer that is “naturally arising” includes any cancer that is not experimentally induced by implantation of cancer cells into a subject, and includes, for example, spontaneously arising cancer, cancer caused by exposure of a patient to a carcinogen(s), cancer resulting from insertion of a transgenic oncogene or knockout of a tumor suppressor gene, and cancer caused by infections, e.g., viral infections. The term “carcinoma” is art recognized and refers to malignancies of epithelial or endocrine tissues. The term also includes carcinosarcomas, which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation. The term “hematopoietic neoplastic disorders” includes diseases involving hyperplastic/neoplastic cells of hematopoietic origin. A hematopoietic neoplastic disorder can arise from myeloid, lymphoid or erythroid lineages, or precursor cells thereof.

As used herein, the term “antibody” refers to any antigen-binding molecule that contains at least one (e.g., one, two, three, four, five, or six) complementary determining region (CDR) (e.g., any of the three CDRs from an immunoglobulin light chain or any of the three CDRs from an immunoglobulin heavy chain) and is capable of specifically binding to an epitope. Non-limiting examples of antibodies include: monoclonal antibodies, polyclonal antibodies, multi-specific antibodies (e.g., bi-specific antibodies), single-chain antibodies, chimeric antibodies, human antibodies, and humanized antibodies. In some embodiments, an antibody can contain an Fc region of a human antibody. The term antibody also includes derivatives, e.g., bi-specific antibodies, single-chain antibodies, and multi-specific antibodies formed from antibody fragments.

As used herein, the term “antigen-binding fragment” refers to a portion of a full-length antibody, wherein the portion of the antibody is capable of specifically binding to an antigen. In some embodiments, the antigen-binding fragment contains at least one variable domain (e.g., a variable domain of a heavy chain or a variable domain of light chain). Non-limiting examples of antibody fragments include, e.g., Fab, Fab′, F(ab′)₂, and Fv fragments.

As used herein, the term “chimeric antibody” refers to an antibody that contains a sequence present in at least two different antibodies (e.g., antibodies from two different mammalian species such as a human and a non-human antibody). A non-limiting example of a chimeric antibody is an antibody containing the variable domain sequences (e.g., all or part of a light chain and/or heavy chain variable domain sequence) of a non-human (e.g., mouse, rabbit) antibody and the constant domains of a human antibody. Additional examples of chimeric antibodies are described herein and are known in the art.

As used herein, the term “humanized antibody” refers to a non-human antibody which contains minimal sequence derived from a non-human (e.g., mouse, rabbit) immunoglobulin and contains sequences derived from a human immunoglobulin. In non-limiting examples, humanized antibodies are human antibodies (recipient antibody) in which hypervariable (e.g., CDR) region residues of the recipient antibody are replaced by hypervariable (e.g., CDR) region residues from a non-human antibody (e.g., a donor antibody), e.g., a mouse, rat, or rabbit antibody, having the desired specificity, affinity, and capacity. In some embodiments, the Fv framework residues of the human immunoglobulin are replaced by corresponding non-human (e.g., mouse, rabbit) immunoglobulin residues. In some embodiments, humanized antibodies may contain residues which are not found in the recipient antibody or in the donor antibody. These modifications can be made to further refine antibody performance. In some embodiments, the humanized antibody contains substantially all of at least one, and typically two, variable domains, in which all or substantially all of the hypervariable loops (CDRs) correspond to those of a non-human (e.g., mouse, rabbit) immunoglobulin and all or substantially all of the framework regions are those of a human immunoglobulin. The humanized antibody can also contain at least a portion of an immunoglobulin constant region (Fc), typically, that of a human immunoglobulin. Humanized antibodies can be produced using molecular biology methods known in the art. Non-limiting examples of methods for generating humanized antibodies are described herein.

As used herein, the terms “subject” and “patient” are used interchangeably throughout the specification and describe an animal, human or non-human, to whom treatment according to the methods of the present invention is provided. Veterinary and non-veterinary applications are contemplated by the present invention. Human patients can be adult humans or juvenile humans (e.g., humans below the age of 18 years old). In addition to humans, patients include but are not limited to mice, rats, hamsters, guinea-pigs, rabbits, ferrets, cats, dogs, and primates. Included are, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

As used herein, when referring to an antibody, the phrases “specifically binding” and “specifically binds” mean that the antibody interacts with its target molecule (e.g., CD79b) preferably to other molecules, because the interaction is dependent upon the presence of a particular structure (i.e., the antigenic determinant or epitope) on the target molecule; in other words, the reagent is recognizing and binding to molecules that include a specific structure rather than to all molecules in general. An antibody that specifically binds to the target molecule may be referred to as a target-specific antibody. For example, an antibody that specifically binds to a CD79b molecule may be referred to as a CD79b-specific antibody or an anti-CD79b antibody.

As used herein, the terms “polypeptide,” “peptide,” and “protein” are used interchangeably to refer to polymers of amino acids of any length of at least two amino acids.

As used herein, the terms “polynucleotide,” “nucleic acid molecule,” and “nucleic acid sequence” are used interchangeably herein to refer to polymers of nucleotides of any length of at least two nucleotides, and include, without limitation, DNA, RNA, DNA/RNA hybrids, and modifications thereof.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 shows ELISA binding affinity of anti-CD79b antibodies to human CD79b recombinant proteins.

FIG. 2 shows ELISA binding affinity of anti-CD79b antibodies to cynomolgus CD79b recombinant proteins.

FIG. 3 show BLI traces of binding of anti-CD79b antibodies to human CD79b recombinant proteins.

FIG. 4 shows relative internalization of anti-CD79b antibodies from the cell surface of Ramos cells.

FIG. 5 shows quantification of anti-CD79b antigen density on malignant B tumor cell and normal B cell surface

FIG. 6 shows binding affinities of anti-CD79b antibodies to BJAB cell.

FIG. 7 shows binding affinities of anti-CD79b antibodies to Ramos cell.

FIG. 8 shows binding affinities of anti-CD79b antibodies to Daudi cell.

FIG. 9 shows binding affinities of anti-CD79b antibodies to SU-DHL-4 cell.

FIG. 10 shows binding affinities of anti-CD79b antibodies to Nalm-6 cell.

FIG. 11 shows binding affinities of anti-CD79b antibodies to Donor 2890.

FIG. 12 shows binding affinities of anti-CD79b antibodies to Donor 2235.

FIG. 13 shows binding affinities of anti-CD79b antibodies to Donor 889.

FIG. 14 shows binding affinities of anti-CD79b antibodies to Donor 356.

FIG. 15 shows binding affinities of anti-CD79b antibodies to CLL Donor 5716.

FIG. 16 shows binding affinities of anti-CD79b antibodies to CLL Donor 0255.

FIGS. 17A-17B show binding of anti-CD79b antibodies to both long and short isoform of CD79b.

FIG. 18 shows ELISA binding of humanized anti-CD79b antibodies to recombinant human CD79b ECD.

FIG. 19 shows binding of humanized anti-CD79b antibodies to cell lines expressing endogenous CD79b.

FIG. 20 lists the CDR sequences of anti-CD79b antibodies as defined by Kabat definition.

FIG. 21 lists the CDR sequences of anti-CD79b antibodies as defined by Chothia definition.

FIG. 22 lists selected amino acid sequences discussed in the disclosure.

### DETAILED DESCRIPTION

The B lymphocyte antigen receptor is a multimeric complex that includes the antigen-specific component, surface immunoglobulin (Ig). Surface Ig non-covalently associates with two other proteins, CD79a and CD79b, which are necessary for expression and function of the B-cell antigen receptor. CD79 is the signaling component of the B-cell receptor and acts as a covalent heterodimer containing CD79a (i.e., Ig-alpha or MB1) and CD79b (i.e., Ig-beta or B29). CD79b contains an extracellular immunoglobulin (Ig) domain, a transmembrane domain, and an intracellular signaling domain, an immunoreceptor tyrosine-based activation motif (ITAM) domain. CD79b expression has been detected on the surface of almost all non-Hodgkin lymphoma (NHL), acute lymphoblastic leukemia (ALL) and chronic lymphocytic leukemia (CLL) patients.

The present disclosure provides examples of antibodies, antigen-binding fragment thereof, that bind to CD79b.

### CD79b

CD79 is composed of CD79a and CD79b components expressed almost exclusively on B cells and B-cell neoplasms. CD79a and CD79b expression precedes immunoglobulin (Ig) heavy-chain gene rearrangement and CD20 expression during B-cell ontogeny and disappears later than CD20 in the late (plasma cell) stage of B-cell differentiation. Therefore, antibodies to CD79a and CD79b are useful in the differential diagnosis of B-cell neoplasms from T-cell neoplasms or myeloid neoplasms, or L and H lymphocyte predominance Hodgkin's lymphoma from classic Hodgkin's lymphoma. In addition, anti-CD79a and anti-CD79b antibodies are useful markers in the diagnosis of precursor B-acute lymphoblastic leukemia (pre-B-ALL) because many of these tumors are negative for other B-cell markers, such as CD20 and CD45RA.

CD79 has been considered as an interesting therapeutic target for antibody as it is physiologically and exclusively expressed on mature B-cell and in the vast majority of B-cell NHLs, including DLBCLs (from 90 to 100%), but also B acute lymphoblastic leukemia (B-ALL), chronic lymphocytic leukemia (CLL), B-cell prolymphocytic leukemia (PLL), splenic lymphoma with villous lymphocytes (SLVL), hairy cell leukemia (HCL), follicular lymphoma (FL) and mantle-cell (MCL) lymphoma.

CD79, in association with a cell surface immunoglobulin (sIg) for antigen recognition, constitutes the B-cell antigen receptor (BCR) complex that plays a critical role in B-cell maturation and activation. CD79 consists of α (CD79a) and β (CD79b) heterodimers, that functions as the signaling component of the BCR. Both subunits of CD79 contain a single extracellular Ig domain, a transmembrane domain, and an intracellular signaling domain that initiate the BCR signaling after antigen binding, ultimately leading to B-cell activation, antigen presentation, cytokine production and cell proliferation and differentiation.

Antigen engagement of the BCR induces its internalization and processing to the major histocompatibility complex class II (MHCII) compartment, a lysosome-like compartment, for the class II antigen presentation by B cells. This intracellular trafficking is of particular interest as the drug is directly delivered to the targeted cells into the lysosomal compartments, enhancing cytotoxic activity, and it allows for the use of more stable linkers that are cleaved in the MHCII compartment.

A detailed review of CD79b and its functions can be found in Chu, Peiguo G., and Daniel A. Arber. “CD79: a review.” Applied Immunohistochemistry & Molecular Morphology 9.2 (2001): 97-106; Bourbon, Estelle, and Gilles Salles. “Polatuzumab vedotin: an investigational anti-CD79b antibody drug conjugate for the treatment of diffuse large B-cell lymphoma.” Expert Opinion on Investigational Drugs 29.10 (2020): 1079-1088; each of which is incorporated by reference in its entirety.

The present disclosure provides several anti-CD79b antibodies, antigen-binding fragments thereof, and methods of using these anti-CD79b antibodies and antigen-binding fragments to inhibit tumor growth, treat cancers, and to treat autoimmune diseases.

### Antibodies and Antigen Binding Fragments

The present disclosure provides anti-CD79b antibodies and antigen-binding fragments thereof. In general, antibodies (also called immunoglobulins) are made up of two classes of polypeptide chains, light chains and heavy chains. A non-limiting antibody of the present disclosure can be an intact, four immunoglobulin chain antibody comprising two heavy chains and two light chains. The heavy chain of the antibody can be of any isotype including IgM, IgG, IgE, IgA, or IgD or sub-isotype including IgG1, IgG2, IgG2a, IgG2b, IgG3, IgG4, IgE1, IgE2, etc. The light chain can be a kappa light chain or a lambda light chain. An antibody can comprise two identical copies of a light chain and two identical copies of a heavy chain. The heavy chains, which each contain one variable domain (or variable region, VH) and multiple constant domains (or constant regions), bind to one another via disulfide bonding within their constant domains to form the “stem” of the antibody. The light chains, which each contain one variable domain (or variable region, VL) and one constant domain (or constant region), each bind to one heavy chain via disulfide binding. The variable region of each light chain is aligned with the variable region of the heavy chain to which it is bound. The variable regions of both the light chains and heavy chains contain three hypervariable regions sandwiched between more conserved framework regions (FR).

These hypervariable regions, known as the complementary determining regions (CDRs), form loops that comprise the principle antigen binding surface of the antibody. The four framework regions largely adopt a beta-sheet conformation and the CDRs form loops connecting, and in some cases forming part of, the beta-sheet structure. The CDRs in each chain are held in close proximity by the framework regions and, with the CDRs from the other chain, contribute to the formation of the antigen-binding region.

Methods for identifying the CDR regions of an antibody by analyzing the amino acid sequence of the antibody are well known, and a number of definitions of the CDRs are commonly used. The Kabat definition is based on sequence variability, and the Chothia definition is based on the location of the structural loop regions. These methods and definitions are described in, e.g., Martin, “Protein sequence and structure analysis of antibody variable domains,” Antibody engineering, Springer Berlin Heidelberg, 2001. 422-439; Abhinandan, et al. “Analysis and improvements to Kabat and structurally correct numbering of antibody variable domains,” Molecular immunology 45.14 (2008): 3832-3839; Wu, T. T. and Kabat, E. A. (1970) J. Exp. Med. 132: 211-250; Martin et al., Methods Enzymol. 203:121-53 (1991); Morea et al., Biophys Chem. 68(1-3):9-16 (October 1997); Morea et al., J Mol Biol. 275(2):269-94 (January 1998); Chothia et al., Nature 342(6252):877-83 (December 1989); Ponomarenko and Bourne, BMC Structural Biology 7:64 (2007); each of which is incorporated herein by reference in its entirety.

The CDRs are important for recognizing an epitope of an antigen. As used herein, an “epitope” is the smallest portion of a target molecule capable of being specifically bound by the antigen binding domain of an antibody. The minimal size of an epitope may be about three, four, five, six, or seven amino acids, but these amino acids need not be in a consecutive linear sequence of the antigen's primary structure, as the epitope may depend on an antigen's three-dimensional configuration based on the antigen's secondary and tertiary structure.

In some embodiments, the antibody is an intact immunoglobulin molecule (e.g., IgG1, IgG2a, IgG2b, IgG3, IgM, IgD, IgE, IgA). The IgG subclasses (IgG1, IgG2, IgG3, and IgG4) are highly conserved, differ in their constant region, particularly in their hinges and upper CH2 domains. The sequences and differences of the IgG subclasses are known in the art, and are described, e.g., in Vidarsson, et al, “IgG subclasses and allotypes: from structure to effector functions.” Frontiers in immunology 5 (2014); Irani, et al. “Molecular properties of human IgG subclasses and their implications for designing therapeutic monoclonal antibodies against infectious diseases.” Molecular immunology 67.2 (2015): 171-182; Shakib, Farouk, ed. The human IgG subclasses: molecular analysis of structure, function and regulation. Elsevier, 2016; each of which is incorporated herein by reference in its entirety.

The antibody can also be an immunoglobulin molecule that is derived from any species (e.g., human, rodent, mouse, camelid, rabbit). Antibodies disclosed herein also include, but are not limited to, polyclonal, monoclonal, monospecific, polyspecific antibodies, and chimeric antibodies that include an immunoglobulin binding domain fused to another polypeptide. The term “antigen binding domain” or “antigen binding fragment” is a portion of an antibody that retains specific binding activity of the intact antibody, i.e., any portion of an antibody that is capable of specific binding to an epitope on the intact antibody's target molecule. It includes, e.g., Fab, Fab′, F(ab′)2, and variants of these fragments. Thus, in some embodiments, an antibody or an antigen binding fragment thereof can be, e.g., a scFv, a Fv, a Fd, a bispecific antibody, a single-chain antibody molecule, a multi-specific antibody formed from antibody fragments, and any polypeptide that includes a binding domain which is, or is homologous to, an antibody binding domain. Non-limiting examples of antigen binding domains include, e.g., the heavy chain and/or light chain CDRs of an intact antibody, the heavy and/or light chain variable regions of an intact antibody, full length heavy or light chains of an intact antibody, or an individual CDR from either the heavy chain or the light chain of an intact antibody.

In some embodiments, the antigen binding fragment can form a part of a chimeric antigen receptor (CAR). In some embodiments, the scFV has one heavy chain variable domain, and one light chain variable domain. In some embodiments, the scFV has two heavy chain variable domains, and two light chain variable domains.

### Anti-CD79b Antibodies and Antigen-Binding Fragments

The disclosure provides antibodies and antigen-binding fragments thereof that specifically bind to CD79b (e.g., human CD79b). The antibodies and antigen-binding fragments described herein are capable of binding to CD79b. These antibodies can be agonists or antagonists to CD79b mediated BCR signaling. In some embodiments, the antibodies and antigen-binding fragments can bind to the extracellular domains of human CD79b.

The disclosure provides e.g., anti-CD79b antibodies 22D10, 23D8, 29C3, 44G2, 48H10, 57B9, the chimeric antibodies thereof, and the humanized antibodies thereof.

The CDR sequences for 22D10, and 22D10 derived antibodies (e.g., humanized antibodies) include CDRs of the heavy chain variable domain, SEQ ID NOs: 9, 11, 13, and CDRs of the light chain variable domain, SEQ ID NOs: 14-16 as defined by Kabat definition. The CDRs can also be defined by Chothia definition. Under the Chothia definition, the CDR sequences of the heavy chain variable domain are set forth in SEQ ID NOs: 10, 12, 13 and CDR sequences of the light chain variable domain are set forth in SEQ ID NOs: 14-16.

Similarly, the CDR sequences for 23D8, and 23D8 derived antibodies include CDRs of the heavy chain variable domain, SEQ ID NOs: 19, 21, 23, and CDRs of the light chain variable domain, SEQ ID NOs: 24-26, as defined by Kabat definition. Under Chothia definition, the CDR sequences of the heavy chain variable domain are set forth in SEQ ID NOs: 20, 22, 23, and CDRs of the light chain variable domain are set forth in SEQ ID NOs: 24-26.

The CDR sequences for 29C3, and 29C3 derived antibodies include CDRs of the heavy chain variable domain, SEQ ID NOs: 29, 31, 33, and CDRs of the light chain variable domain, SEQ ID NOs: 34-36, as defined by Kabat definition. Under Chothia definition, the CDR sequences of the heavy chain variable domain are set forth in SEQ ID NOs: 30, 32, 33, and CDRs of the light chain variable domain are set forth in SEQ ID NOs: 34-36.

The CDR sequences for 44G2, and 44G2 derived antibodies include CDRs of the heavy chain variable domain, SEQ ID NOs: 39, 41, 43, and CDRs of the light chain variable domain, SEQ ID NOs: 44-46, as defined by Kabat definition. Under Chothia definition, the CDR sequences of the heavy chain variable domain are set forth in SEQ ID NOs: 40, 42, 43, and CDRs of the light chain variable domain are set forth in SEQ ID NOs: 44-46.

The CDR sequences for 48H10, and 48H10 derived antibodies include CDRs of the heavy chain variable domain, SEQ ID NOs: 49, 51, 53, and CDRs of the light chain variable domain, SEQ ID NOs: 54-56, as defined by Kabat definition. Under Chothia definition, the CDR sequences of the heavy chain variable domain are set forth in SEQ ID NOs: 50, 52, 53, and CDRs of the light chain variable domain are set forth in SEQ ID NOs: 54-56.

The CDR sequences for 57B9, and 57B9 derived antibodies include CDRs of the heavy chain variable domain, SEQ ID NOs: 59, 61, 63, and CDRs of the light chain variable domain, SEQ ID NOs: 64-66, as defined by Kabat definition. Under Chothia definition, the CDR sequences of the heavy chain variable domain are set forth in SEQ ID NOs: 60, 62, 63, and CDRs of the light chain variable domain are set forth in SEQ ID NOs: 64-66.

The amino acid sequence for the heavy chain variable region of 22D10 antibody is set forth in SEQ ID NO: 7. The amino acid sequence for the light chain variable region of 22D10 antibody is set forth in SEQ ID NO: 8.

The amino acid sequence for the heavy chain variable region of 23D8 antibody is set forth in SEQ ID NO: 17. The amino acid sequence for the light chain variable region of 23D8 antibody is set forth in SEQ ID NO: 18.

The amino acid sequence for the heavy chain variable region of 29C3 antibody is set forth in SEQ ID NO: 27. The amino acid sequence for the light chain variable region of 29C3 antibody is set forth in SEQ ID NO: 28.

The amino acid sequence for the heavy chain variable region of 44G2 antibody is set forth in SEQ ID NO: 37. The amino acid sequence for the light chain variable region of 44G2 antibody is set forth in SEQ ID NO: 38.

The amino acid sequence for the heavy chain variable region of 48H10 antibody is set forth in SEQ ID NO: 47. The amino acid sequence for the light chain variable region of 48H10 antibody is set forth in SEQ ID NO: 48.

The amino acid sequence for the heavy chain variable region of 57B9 antibody is set forth in SEQ ID NO: 57. The amino acid sequence for the light chain variable region of 57B9 antibody is set forth in SEQ ID NO: 58.

In some embodiments, the anti-CD79b antibodies are humanized antibodies. In some embodiments, the anti-CD79b antibodies are humanized 23D8, 44G2, 48H10, or 57B9.

The amino acid sequence for the heavy chain variable region of the humanized 23D8 antibody is set forth in SEQ ID NO: 69. The amino acid sequence for the light chain variable region of 23D8 antibody is set forth in SEQ ID NO: 70.

The amino acid sequence for the heavy chain variable region of the humanized 44G2 antibody is set forth in SEQ ID NO: 71. The amino acid sequence for the light chain variable region of 44G2 antibody is set forth in SEQ ID NO: 72.

The amino acid sequence for the heavy chain variable region of the humanized 48H10 antibody is set forth in SEQ ID NO: 73. The amino acid sequence for the light chain variable region of 48H10 antibody is set forth in SEQ ID NO: 74.

The amino acid sequence for the heavy chain variable region of the humanized 57B9 antibody is set forth in SEQ ID NO: 75. The amino acid sequence for the light chain variable region of 57B9 antibody is set forth in SEQ ID NO: 76.

The amino acid sequences for heavy chain variable regions and light variable regions of the modified antibodies are also provided. In some embodiments, the heavy chain variable region is at least 80%, 85%, 90%, 91%, 92%, 93%, 94%, 95%, 96%, 97%, 98%, or 99% identical to any one of SEQ ID NOs: 7, 17, 27, 37, 47, 57, 69, 71, 73, and 75. In some embodiments, the light chain variable region is at least 80%, 85%, 90%, 91%, 92%, 93%, 94%, 95%, 96%, 97%, 98%, or 99% identical to any one of SEQ ID NOs: 8, 18, 28, 38, 48, 58, 70, 72, 74 and 76. The heavy chain variable region sequence can be paired with the corresponding light chain variable region sequence, and together they bind to CD79b.

Humanization percentage means the percentage identity of the heavy chain or light chain variable region sequence as compared to human antibody sequences in International Immunogenetics Information System (IMGT) database. In some embodiments, humanization percentage is greater than 80%, 81%, 82%, 83%, 84%, 85%, 86%, 87%, 88% 89%, 90%, 91%, 92%, 93%, 94%, or 95%. A detailed description regarding how to determine humanization percentage and how to determine top hits is known in the art, and is described, e.g., in Jones, et al. “The INNs and outs of antibody nonproprietary names.” MAbs. Vol. 8. No. 1. Taylor & Francis, 2016, which is incorporated herein by reference in its entirety. A high humanization percentage often has various advantages, e.g., more safe and more effective in humans, more likely to be tolerated by a human subject, and/or less likely to have side effects. In some embodiments, the variable regions are fully human, e.g., derived from human heavy chain immunoglobulin locus sequences (e.g., recombination of human IGHV, human IGHD, and human IGHJ genes), and/or human kappa chain immunoglobulin locus sequences (e.g., recombination of human IGKV and human IGKJ genes).

Furthermore, in some embodiments, the antibodies or antigen-binding fragments thereof described herein can also contain one, two, or three heavy chain variable region CDRs selected from the group of SEQ ID NOs: 9, 11, 13, SEQ ID NOs: 19, 21, 23, SEQ ID NOs: 29, 31, 33, SEQ ID NOs: 39, 41, 43, SEQ ID NOs: 49, 51, 53, SEQ ID NOs: 59, 61, 63, SEQ ID NOs: 10, 12, 13, SEQ ID NOs: 20, 22, 23, SEQ ID NOs: 30, 32, 33, SEQ ID NOs: 40, 42, 43, SEQ ID NOs: 50, 52, 53, SEQ ID NOs: 60, 62, 63; and/or one, two, or three light chain variable region CDRs selected from the group of SEQ ID NOs: 14-16, SEQ ID NOs: 24-26, SEQ ID NOs: 34-36, SEQ ID NOs: 44-46, SEQ ID NOs: 54-56 and SEQ ID NOs: 64-66.

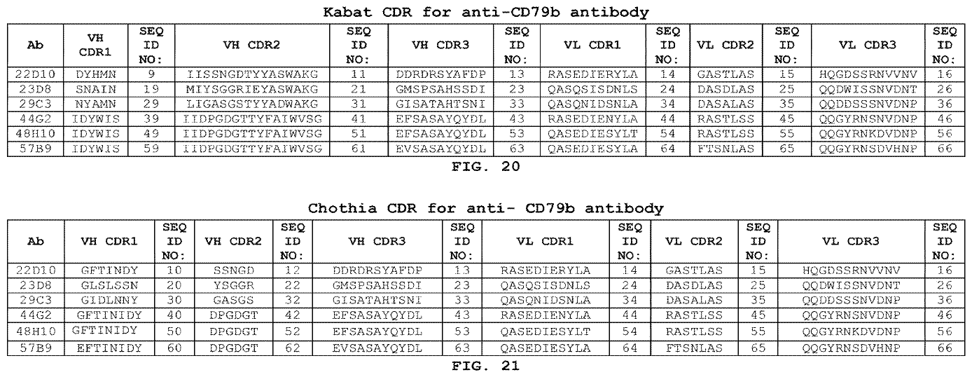

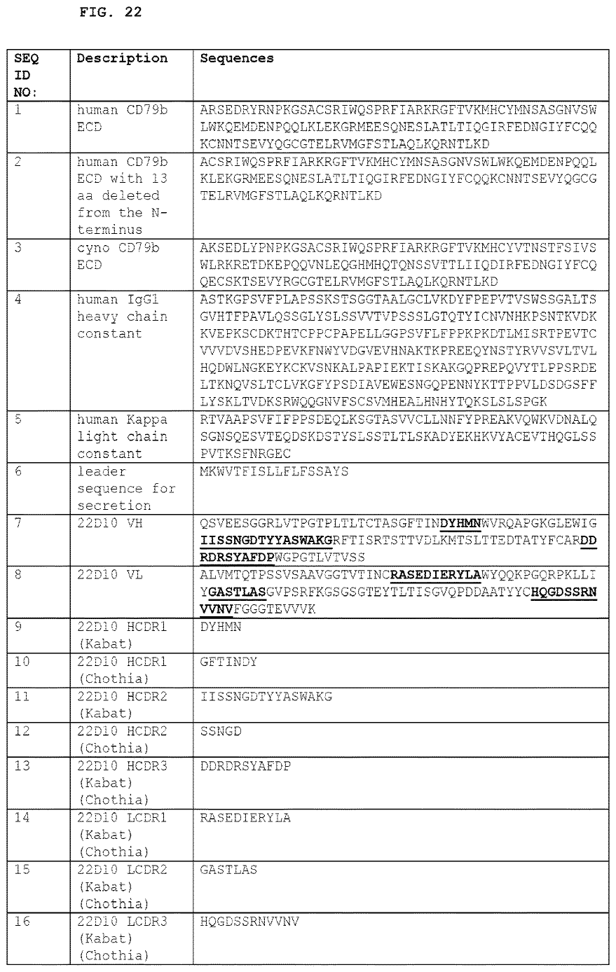

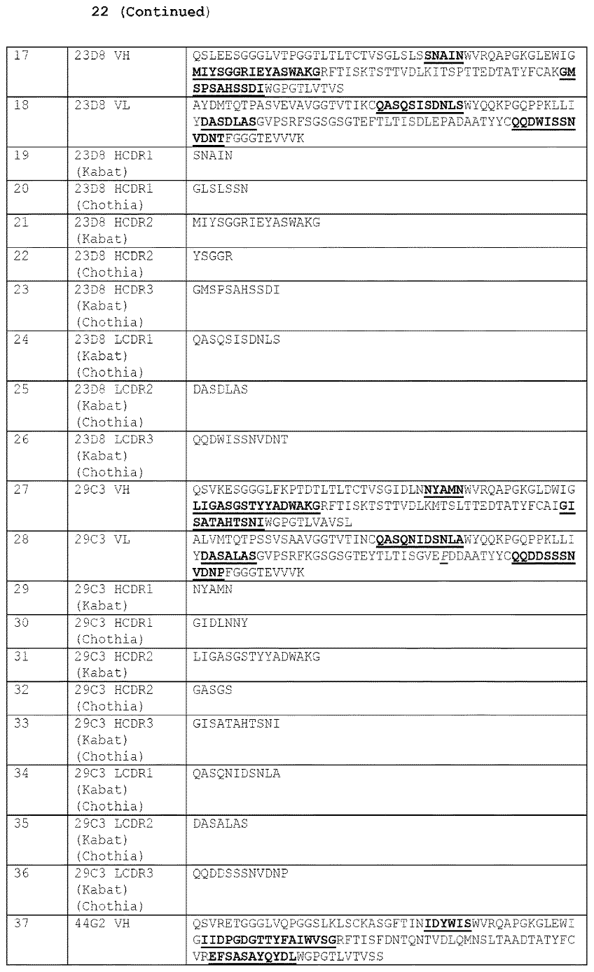

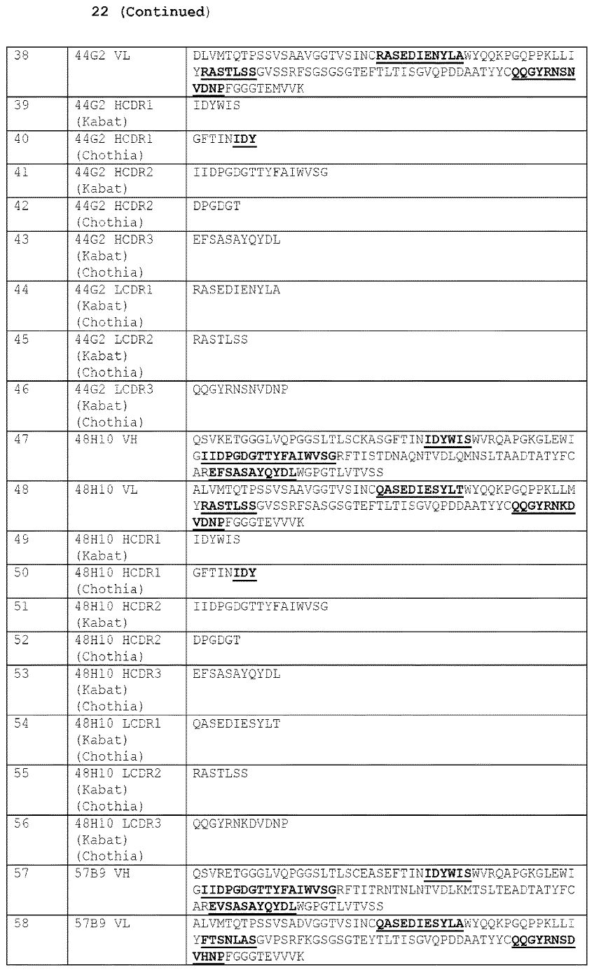

In some embodiments, the antibodies can have a heavy chain variable region (VH) comprising complementarity determining regions (CDRs) 1, 2, 3, wherein the CDR1 region comprises or consists of an amino acid sequence that is at least 80%, 85%, 90%, or 95% identical to a selected VH CDR1 amino acid sequence, the CDR2 region comprises or consists of an amino acid sequence that is at least 80%, 85%, 90%, or 95% identical to a selected VH CDR2 amino acid sequence, and the CDR3 region comprises or consists of an amino acid sequence that is at least 80%, 85%, 90%, or 95% identical to a selected VH CDR3 amino acid sequence. In some embodiments, the antibody can have a light chain variable region (VL) comprising CDRs 1, 2, 3, wherein the CDR1 region comprises or consists of an amino acid sequence that is at least 80%, 85%, 90%, or 95% identical to a selected VL CDR1 amino acid sequence, the CDR2 region comprises or consists of an amino acid sequence that is at least 80%, 85%, 90%, or 95% identical to a selected VL CDR2 amino acid sequence, and the CDR3 region comprises or consists of an amino acid sequence that is at least 80%, 85%, 90%, or 95% identical to a selected VL CDR3 amino acid sequence. The selected VH CDRs 1, 2, 3 amino acid sequences and the selected VL CDRs, 1, 2, 3 amino acid sequences are shown in FIG. 20(Kabat CDR) and FIG. 21 (Chothia CDR).

In some embodiments, the antibody or an antigen-binding fragment described herein can contain a heavy chain variable domain containing one, two, or three of the CDRs of SEQ ID NO: 9 with zero, one or two amino acid insertions, deletions, or substitutions; SEQ ID NO: 11 with zero, one or two amino acid insertions, deletions, or substitutions; SEQ ID NO: 13 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 19 with zero, one or two amino acid insertions, deletions, or substitutions; SEQ ID NO: 21 with zero, one or two amino acid insertions, deletions, or substitutions; SEQ ID NO: 23 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 29 with zero, one or two amino acid insertions, deletions, or substitutions; SEQ ID NO: 31 with zero, one or two amino acid insertions, deletions, or substitutions; SEQ ID NO: 33 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 39 with zero, one or two amino acid insertions, deletions, or substitutions; SEQ ID NO: 41 with zero, one or two amino acid insertions, deletions, or substitutions; SEQ ID NO: 43 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 49 with zero, one or two amino acid insertions, deletions, or substitutions; SEQ ID NO: 51 with zero, one or two amino acid insertions, deletions, or substitutions; SEQ ID NO: 53 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 59 with zero, one or two amino acid insertions, deletions, or substitutions; SEQ ID NO: 61 with zero, one or two amino acid insertions, deletions, or substitutions; SEQ ID NO: 63 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 10 with zero, one or two amino acid insertions, deletions, or substitutions; SEQ ID NO: 12 with zero, one or two amino acid insertions, deletions, or substitutions; SEQ ID NO: 13 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 20 with zero, one or two amino acid insertions, deletions, or substitutions; SEQ ID NO: 22 with zero, one or two amino acid insertions, deletions, or substitutions; SEQ ID NO: 23 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 30 with zero, one or two amino acid insertions, deletions, or substitutions; SEQ ID NO: 32 with zero, one or two amino acid insertions, deletions, or substitutions; SEQ ID NO: 33 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 40 with zero, one or two amino acid insertions, deletions, or substitutions; SEQ ID NO: 42 with zero, one or two amino acid insertions, deletions, or substitutions; SEQ ID NO: 43 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 50 with zero, one or two amino acid insertions, deletions, or substitutions; SEQ ID NO: 52 with zero, one or two amino acid insertions, deletions, or substitutions; SEQ ID NO: 53 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 60 with zero, one or two amino acid insertions, deletions, or substitutions; SEQ ID NO: 62 with zero, one or two amino acid insertions, deletions, or substitutions; SEQ ID NO: 63 with zero, one or two amino acid insertions, deletions, or substitutions.

In some embodiments, the antibody or an antigen-binding fragment described herein can contain a light chain variable domain containing one, two, or three of the CDRs of SEQ ID NO: 14 with zero, one or two amino acid insertions, deletions, or substitutions; SEQ ID NO: 15 with zero, one or two amino acid insertions, deletions, or substitutions; SEQ ID NO: 16 with zero, one or two amino acid insertions, deletions, or substitutions.

In some embodiments, the antibody or an antigen-binding fragment described herein can contain a light chain variable domain containing one, two, or three of the CDRs of SEQ ID NO: 24 with zero, one or two amino acid insertions, deletions, or substitutions; SEQ ID NO: 25 with zero, one or two amino acid insertions, deletions, or substitutions; SEQ ID NO: 26 with zero, one or two amino acid insertions, deletions, or substitutions.

In some embodiments, the antibody or an antigen-binding fragment described herein can contain a light chain variable domain containing one, two, or three of the CDRs of SEQ ID NO: 34 with zero, one or two amino acid insertions, deletions, or substitutions; SEQ ID NO: 35 with zero, one or two amino acid insertions, deletions, or substitutions; SEQ ID NO: 36 with zero, one or two amino acid insertions, deletions, or substitutions.

In some embodiments, the antibody or an antigen-binding fragment described herein can contain a light chain variable domain containing one, two, or three of the CDRs of SEQ ID NO: 44 with zero, one or two amino acid insertions, deletions, or substitutions; SEQ ID NO: 45 with zero, one or two amino acid insertions, deletions, or substitutions; SEQ ID NO: 46 with zero, one or two amino acid insertions, deletions, or substitutions.

In some embodiments, the antibody or an antigen-binding fragment described herein can contain a light chain variable domain containing one, two, or three of the CDRs of SEQ ID NO: 54 with zero, one or two amino acid insertions, deletions, or substitutions; SEQ ID NO: 55 with zero, one or two amino acid insertions, deletions, or substitutions; SEQ ID NO: 56 with zero, one or two amino acid insertions, deletions, or substitutions.

In some embodiments, the antibody or an antigen-binding fragment described herein can contain a light chain variable domain containing one, two, or three of the CDRs of SEQ ID NO: 64 with zero, one or two amino acid insertions, deletions, or substitutions; SEQ ID NO: 65 with zero, one or two amino acid insertions, deletions, or substitutions; SEQ ID NO: 66 with zero, one or two amino acid insertions, deletions, or substitutions.

The insertions, deletions, and substitutions can be within the CDR sequence, or at one or both terminal ends of the CDR sequence. In some embodiments, the CDR is determined based on Kabat definition. In some embodiments, the CDR is determined based on Chothia definition. In some embodiments, the CDR is determined based on a combination of Kabat definition and Chothia definition.

The disclosure also provides antibodies or antigen-binding fragments thereof that bind to CD79b. The antibodies or antigen-binding fragments thereof contain a heavy chain variable region (VH) comprising or consisting of an amino acid sequence that is at least 80%, 85%, 90%, 95%, 96%, 97%, 98%, or 99% identical to a selected VH sequence, and a light chain variable region (VL) comprising or consisting of an amino acid sequence that is at least 80%, 85%, 90%, 95%, 96%, 97%, 98%, or 99% identical to a selected VL sequence. In some embodiments, the selected VH sequence is SEQ ID NO: 7, and the selected VL sequence is SEQ ID NO: 8. In some embodiments, the selected VH sequence is SEQ ID NO: 17 and the selected VL sequence is SEQ ID NO: 18. In some embodiments, the selected VH sequence is SEQ ID NO: 27, and the selected VL sequence is SEQ ID NO: 28. In some embodiments, the selected VH sequence is SEQ ID NO: 37, and the selected VL sequence is SEQ ID NO: 38. In some embodiments, the selected VH sequence is SEQ ID NO: 47, and the selected VL sequence is SEQ ID NO: 48. In some embodiments, the selected VH sequence is SEQ ID NO: 57, and the selected VL sequence is SEQ ID NO: 58. In some embodiments, the selected VH sequence is SEQ ID NO: 69, and the selected VL sequence is SEQ ID NO: 70. In some embodiments, the selected VH sequence is SEQ ID NO: 71, and the selected VL sequence is SEQ ID NO: 72. In some embodiments, the selected VH sequence is SEQ ID NO: 73, and the selected VL sequence is SEQ ID NO: 74. In some embodiments, the selected VH sequence is SEQ ID NO: 75, and the selected VL sequence is SEQ ID NO: 76.

The disclosure also provides antibodies or antigen-binding fragments thereof that can compete with the antibodies described herein. In some aspects, the antibodies or antigen-binding fragments can bind to the same epitope as the antibodies described herein.

The present disclosure also provides an antibody or antigen-binding fragment thereof that cross-competes with any antibody or antigen-binding fragment as described herein. The cross-competing assay is known in the art, and is described e.g., in Moore et al., “Antibody cross-competition analysis of the human immunodeficiency virus type 1 gp120 exterior envelope glycoprotein.” Journal of virology 70.3 (1996): 1863-1872, which is incorporated herein reference in its entirety. In one aspect, the present disclosure also provides an antibody or antigen-binding fragment thereof that binds to the same epitope or region as any antibody or antigen-binding fragment as described herein. The epitope binning assay is known in the art, and is described e.g., in Estep et al. “High throughput solution-based measurement of antibody-antigen affinity and epitope binning.” MAbs. Vol. 5. No. 2. Taylor & Francis, 2013, which is incorporated herein reference in its entirety.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For example, the comparison of sequences and determination of percent identity between two sequences can be accomplished using a Blossum 62 scoring matrix with a gap penalty of 12, a gap extend penalty of 4, and a frameshift gap penalty of 5.

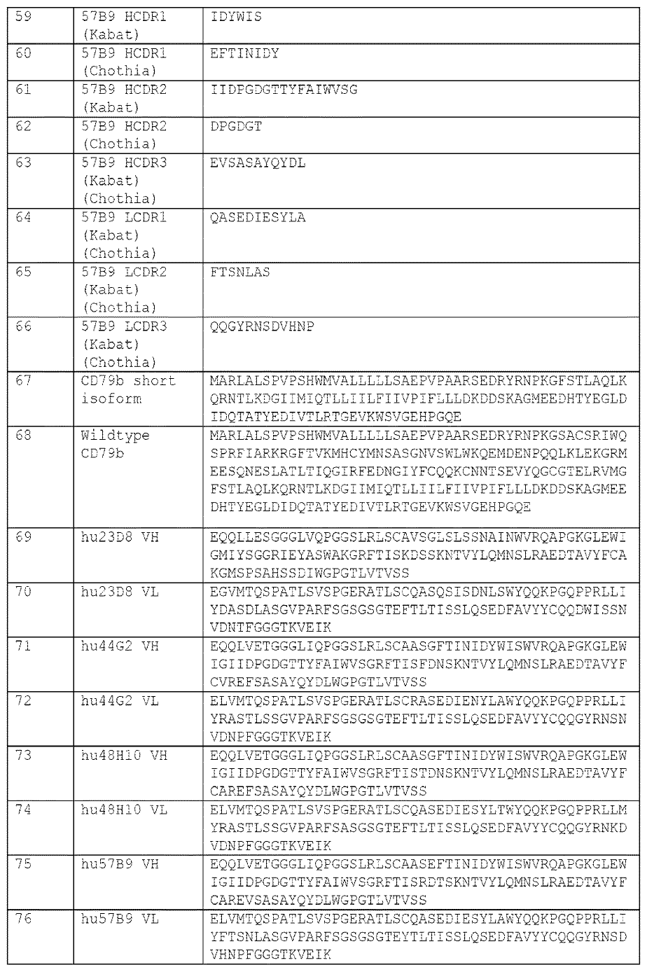

The disclosure also provides nucleic acid comprising a polynucleotide encoding a polypeptide comprising an immunoglobulin heavy chain or an immunoglobulin light chain. The immunoglobulin heavy chain or immunoglobulin light chain comprises CDRs as shown in FIG. 20 or FIG. 21, or have sequences as shown in FIG. 22. When the polypeptides are paired with corresponding polypeptide (e.g., a corresponding heavy chain variable region or a corresponding light chain variable region), the paired polypeptides bind to CD79b (e.g., human CD79b).

The anti-CD79b antibodies and antigen-binding fragments can also be antibody variants (including derivatives and conjugates) of antibodies or antibody fragments and multi-specific (e.g., bi-specific) antibodies or antibody fragments. Additional antibodies provided herein are polyclonal, monoclonal, multimeric, multi-specific (e.g., bi-specific), humanized antibodies, chimeric antibodies (e.g., human-mouse chimera), single-chain antibodies, intracellularly-made antibodies (i.e., intrabodies), and antigen-binding fragments thereof. The antibodies or antigen-binding fragments thereof can be of any type (e.g., IgG, IgE, IgM, IgD, IgA, and IgY), class (e.g., IgG1, IgG2, IgG3, IgG4, IgA1, and IgA2), or subclass. In some embodiments, the antibody or antigen-binding fragment thereof is an IgG antibody or antigen-binding fragment thereof.

Fragments of antibodies are suitable for use in the methods provided so long as they retain the desired affinity and specificity of the full-length antibody. Thus, a fragment of an antibody that binds to CD79b will retain an ability to bind to CD79b. An Fv fragment is an antibody fragment which contains a complete antigen recognition and binding site. This region consists of a dimer of one heavy and one light chain variable domain in tight association, which can be covalent in nature, for example in scFv. It is in this configuration that the three CDRs of each variable domain interact to define an antigen binding site on the surface of the VH-VL dimer. Collectively, the six CDRs or a subset thereof confer antigen binding specificity to the antibody. However, even a single variable domain (or half of an Fv comprising only three CDRs specific for an antigen) can have the ability to recognize and bind antigen, although usually at a lower affinity than the entire binding site.

Single-chain Fv or (scFv) antibody fragments comprise the VH and VL domains (or regions) of antibody, wherein these domains are present in a single polypeptide chain. Generally, the scFv polypeptide further comprises a polypeptide linker between the VH and VL domains, which enables the scFv to form the desired structure for antigen binding.

The Fab fragment contains a variable and constant domain of the light chain and a variable domain and the first constant domain (CH1) of the heavy chain. F(ab′)2 antibody fragments comprise a pair of Fab fragments which are generally covalently linked near their carboxy termini by hinge cysteines between them. Other chemical couplings of antibody fragments are also known in the art.

Antibodies and antibody fragments of the present disclosure can be modified in the Fc region to provide desired effector functions or serum half-life. In some embodiments, the Fc region can be modified to silence or decrease complement-dependent cytotoxicity (CDC) or antibody-dependent cellular cytotoxicity (ADCC).

In some embodiments, the multi-specific antibody is a bi-specific antibody. Bi-specific antibodies can be made by engineering the interface between a pair of antibody molecules to maximize the percentage of heterodimers that are recovered from recombinant cell culture. For example, the interface can contain at least a part of the CH3 domain of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers. This method is described, e.g., in WO 96/27011, which is incorporated by reference in its entirety.

Any of the antibodies or antigen-binding fragments described herein may be conjugated to a stabilizing molecule (e.g., a molecule that increases the half-life of the antibody or antigen-binding fragment thereof in a subject or in solution). Non-limiting examples of stabilizing molecules include: a polymer (e.g., a polyethylene glycol) or a protein (e.g., serum albumin, such as human serum albumin). The conjugation of a stabilizing molecule can increase the half-life or extend the biological activity of an antibody or an antigen-binding fragment in vitro (e.g., in tissue culture or when stored as a pharmaceutical composition) or in vivo (e.g., in a human).

In some embodiments, the antibodies or antigen-binding fragments described herein can be conjugated to a therapeutic agent. The antibody-drug conjugate comprising the antibody or antigen-binding fragment thereof can covalently or non-covalently bind to a therapeutic agent. In some embodiments, the therapeutic agent is a cytotoxic or cytostatic agent (e.g., cytochalasin B, gramicidin D, ethidium bromide, emetine, mitomycin, etoposide, tenoposide, vincristine, vinblastine, colchicin, doxorubicin, daunorubicin, dihydroxy anthracin, maytansinoids such as DM-1 and DM-4, dione, mitoxantrone, mithramycin, actinomycin D, 1-dehydrotestosterone, glucocorticoids, procaine, tetracaine, lidocaine, propranolol, puromycin, epirubicin, and cyclophosphamide and analogs).

In some embodiments, the antibody or antigen-binding fragment thereof described herein recognizes an endogenous CD79b or a recombinant CD79b. In some embodiments, the antibody or antigen-binding fragment thereof described herein recognizes human CD79b (e.g., the extracellular region of human CD79b).

### Antibody Drug Conjugates (ADC)

The antibodies, the antigen-binding fragments thereof, or the antigen-binding protein constructs (e.g., bispecific antibodies) described herein can be conjugated to a therapeutic agent (a drug). The therapeutic agent can be covalently or non-covalently bind to the antibody or antigen-binding fragment or the antigen binding protein construct (e.g., a bispecific antibody).

In some embodiments, the therapeutic agent is a cytotoxic or cytostatic agent (e.g., monomethyl auristatin E, monomethyl auristatin F, cytochalasin B, gramicidin D, ethidium bromide, emetine, mitomycin, etoposide, tenoposide, vincristine, vinblastine, colchicin, doxorubicin, daunorubicin, dihydroxy anthracin, maytansinoids such as DM-1 and DM-4, dione, mitoxantrone, mithramycin, actinomycin D, 1-dehydrotestosterone, glucocorticoids, procaine, tetracaine, lidocaine, propranolol, puromycin, epirubicin, and cyclophosphamide and analogs). Useful classes of cytotoxic, cytostatic, or immunomodulatory agents include, for example, antitubulin agents, DNA minor groove binders, DNA replication inhibitors, and alkylating agents.

In some embodiments, the therapeutic agent can include, but not limited to, cytotoxic reagents, such as chemo-therapeutic agents, immunotherapeutic agents and the like, antiviral agents or antimicrobial agents. In some embodiments, the therapeutic agent to be conjugated can be selected from, but not limited to, MMAE (monomethyl auristatin E), MMAD (monomethyl auristatin D), or MMAF (monomethyl auristatin F).

In some embodiments, the therapeutic agent is an auristatin, such as auristatin E (also known in the art as a derivative of dolastatin-10) or a derivative thereof. The auristatin can be, for example, an ester formed between auristatin E and a keto acid. For example, auristatin E can be reacted with paraacetyl benzoic acid or benzoylvaleric acid to produce AEB and AEVB, respectively. Other typical auristatins include AFP, MMAF, and MMAE. The synthesis and structure of exemplary auristatins are described in U.S. Patent Application Publication No. 2003-0083263; International Patent Publication No. WO 04/010957, International Patent Publication No. WO 02/088172, and U.S. Pat. Nos. 7,498,298, 6,884,869, 6,323,315; 6,239,104; 6,034,065; 5,780,588; 5,665,860; 5,663,149; 5,635,483; 5,599,902; 5,554,725; 5,530,097; 5,521,284; 5,504,191; 5,410,024; 5,138,036; 5,076,973; 4,986,988; 4,978,744; 4,879,278; 4,816,444; and 4,486,414, each of which is incorporated by reference herein in its entirety and for all purposes.

Auristatins have been shown to interfere with microtubule dynamics and nuclear and cellular division and have anticancer activity. Auristatins bind tubulin and can exert a cytotoxic or cytostatic effect on cancer cell. There are a number of different assays, known in the art, which can be used for determining whether an auristatin or resultant antibody-drug conjugate exerts a cytostatic or cytotoxic effect on a desired cell.

In some embodiments, the therapeutic agent is a chemotherapeutic agent. Examples of chemotherapeutic agents include alkylating agents such as thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfornithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK7; razoxane; sizofiran; spirogermanium; tenuazonic acid; triaziquone; 2′,2′,2′-trichlorotriethylamine; urethan; vindesine; dacarbazine; mannomustine; mitobronitol; mitolactol; pipobroman; gacytosine; arabinoside (“Ara-C”); cyclophosphamide; taxanes, e.g. paclitaxel (TAXOL®, Bristol-Myers Squibb Oncology, Princeton, N.J.) and doxetaxel (TAXOTERE®, Rhone-Poulenc Rorer, Antony, France); chlorambucil; gemcitabine; 6-thioguanine; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above. A detailed description of the chemotherapeutic agents can be found in, e.g., US20180193477A1, which is incorporated by reference in its entirety.

In some embodiments, the antigen-binding construct is coupled to the drug via a cleavable linker e.g. a SPBD linker or a maleimidocaproyl-valine-citrulline-p-aminobenzyloxycarbonyl (VC) linker. In some embodiments, the antigen-binding construct is coupled to the drug via a non-cleavable linker e.g. a MCC linker formed using SMCC or sulfo-SMCC. Selection of an appropriate linker for a given ADC can be readily made by the skilled person having knowledge of the art and taking into account relevant factors, such as the site of attachment to the antigen binding construct, any structural constraints of the drug and the hydrophobicity of the drug (see, for example, review in Nolting, Chapter 5, Antibody-Drug Conjugates: Methods in Molecular Biology, 2013, Ducry (Ed.), Springer). A number of specific linker-toxin combinations have been described and may be used with the antigen binding constructs described herein to prepare ADCs in certain embodiments. Examples include, but are not limited to, cleavable peptide-based linkers with auristatins such as MMAE and MMAF, camptothecins such as SN-38, duocarmycins and PBD dimers; non-cleavable MC-based linkers with auristatins MMAF and MMAE; acid-labile hydrazone-based linkers with calicheamicins and doxorubicin; disulfide-based linkers with maytansinoids such as DM1 and DM4, and bis-maleimido-trioxyethylene glycol (BMPEO)-based linkers with maytansinoid DM1. Some these therapeutic agents and linkers are described, e.g., in Peters & Brown, (2015) Biosci. Rep. e00225; Dosio et al., (2014) Recent Patents on Anti-Cancer Drug Discovery 9:35-65; US Patent Publication No. US 2015/0374847, and US20180193477A1; which are incorporated herein by reference in the entirety.

Depending on the desired drug and selected linker, those skilled in the art can select suitable method for coupling them together. For example, some conventional coupling methods, such as amine coupling methods, can be used to form the desired drug-linker complex which still contains reactive groups for conjugating to the antibodies through covalent linkage. In some embodiments, a drug-maleimide complex (i.e., maleimide linking drug) can be used for the payload bearing reactive group in the present disclosure. Most common reactive group capable of bonding to thiol group in ADC preparation is maleimide. Additionally, organic bromides, iodides also are frequently used.

The ADC can be prepared by one of several routes known in the art, employing organic chemistry reactions, conditions, and reagents known to those skilled in the art (see, for example, Bioconjugate Techniques (G. T. Hermanson, 2013, Academic Press). For example, conjugation can be achieved by (1) reaction of a nucleophilic group or an electrophilic group of an antibody with a bivalent linker reagent, to form antibody-linker intermediate Ab-L, via a covalent bond, followed by reaction with an activated drug moiety D; or (2) reaction of a nucleophilic group or an electrophilic group of a drug moiety with a linker reagent, to form drug-linker intermediate D-L, via a covalent bond, followed by reaction with the nucleophilic group or an electrophilic group of an antibody. Conjugation methods (1) and (2) can be employed with a variety of antibodies, drug moieties, and linkers to prepare the ADCs described here. Various prepared linkers, linker components and toxins are commercially available or may be prepared using standard synthetic organic chemistry techniques. These methods are described e.g., in March's Advanced Organic Chemistry (Smith & March, 2006, Sixth Ed., Wiley); Toki et al., (2002) J. Org. Chem. 67:1866-1872; Frisch et al., (1997) Bioconj. Chem. 7:180-186; Bioconjugate Techniques (G. T. Hermanson, 2013, Academic Press); US20210379193A1, and US20180193477A1, which are incorporated herein by reference in the entirety. In addition, a number of pre-formed drug-linkers suitable for reaction with a selected antigen binding construct are also available commercially, for example, linker-toxins comprising DM1, DM4, MMAE, MMAF or Duocarmycin SA are available from Creative BioLabs (Shirley, N.Y.).

Several specific examples of methods of preparing ADCs are known in the art and are described in U.S. Pat. No. 8,624,003 (pot method), U.S. Pat. No. 8,163,888 (one-step), and U.S. Pat. No. 5,208,020 (two-step method), and US20180193477A1, which are incorporated herein by reference in the entirety. Other methods are known in the art and include those described in Antibody-Drug Conjugates: Methods in Molecular Biology, 2013, Ducry (Ed.), Springer.

Drug loading is represented by the number of drug moieties per antibody in a molecule of ADC. For some antibody-drug conjugates, the drug loading may be limited by the number of attachment sites on the antibody. For example, where the attachment is a cysteine thiol, as in certain exemplary embodiments described herein, the drug loading may range from 0 to 8 drug moieties per antibody. In certain embodiments, higher drug loading, e.g. p & 5, may cause aggregation, insolubility, toxicity, or loss of cellular permeability of certain antibody-drug conjugates. In certain embodiments, the average drug loading for an antibody-drug conjugate ranges from 1 to about 8; from about 2 to about 6; or from about 3 to about 5. Indeed, it has been shown that for certain antibody-drug conjugates, the optimal ratio of drug moieties per antibody can be around 4. In some embodiments, the drug-to-antibody ratio (DAR) is about or at least 1, 2, 3, 4, 5, 6, 7, or 8. In some embodiments, the average DAR in the composition is about 1~about 2, about 2~about 3, about 3~about 4, about 3~about 5, about 4~about 5, about 5~about 6, about 6~about 7, or about 7~about 8.

### Antibody and ADC Characteristics

The antibodies or antigen-binding fragments thereof as described herein or ADC derived therefrom can be an agonist or antagonist. In some embodiments, by binding to CD79b, the antibody can inhibit CD79b mediated BCR signaling.

In some implementations, the antibody (or antigen-binding fragments thereof) or ADC derived therefrom specifically binds to CD79b (e.g., human CD79b, monkey CD79b (e.g., rhesus macaques, Macaca fascicularis), dog CD79b, mouse CD79b) with a dissociation rate (koff) of less than 0.1 s⁻¹, less than 0.01 s⁻¹, less than 0.001 s⁻¹, less than 0.0001 s⁻¹, less than 0.00001 s⁻¹, less than 0.000001 s⁻¹ or less than 0.0000001 s⁻¹. In some embodiments, the dissociation rate (koff) is greater than 0.01 s⁻¹, greater than 0.001 s⁻¹, greater than 0.0001 s⁻¹, greater than 0.00001 s⁻¹, greater than 0.000001 s⁻¹, greater than 0.0000001 s⁻¹ or greater than 0.00000001 s⁻¹.

In some embodiments, kinetic association rates (kon) is greater than 1×10²/Ms, greater than 1×10³/Ms, greater than 1×10⁴/Ms, greater than 1×10⁵/Ms, or greater than 1×10⁶/Ms. In some embodiments, kinetic association rates (kon) is less than 1×10⁵/Ms, less than 1×10⁶/Ms, or less than 1×10⁷/Ms.

Affinities can be deduced from the quotient of the kinetic rate constants (KD=koff/kon). In some embodiments, KD is less than 1×10⁻⁶ M, less than 1×10⁻⁷ M, less than 1×10⁻⁸ M, less than 1×10⁻⁹ M, less than 1×10⁻¹⁰ M, less than 1×10⁻¹¹ M, less than 1×10⁻¹² M, less than 1×10⁻¹³ M or less than 1×10⁻¹⁴ M. In some embodiments, the KD is less than 50 nM, 30 nM, 20 nM, 15 nM, 10 nM, 9 nM, 8 nM, 7 nM, 6 nM, 5 nM, 4 nM, 3 nM, 2 nM, or 1 nM. In some embodiments, KD is greater than 1×10⁻⁷M, greater than 1×10⁻⁸ M, greater than 1×10⁻⁹ M, greater than 1×10⁻¹⁰ M, greater than 1×10⁻¹¹ M, greater than 1×10⁻¹² M, greater than 1×10⁻¹³ M, greater than 1×10⁻¹⁴ M.

General techniques for measuring the affinity of an antibody for an antigen include, e.g., BLI, ELISA, RIA, flow cytometry, and surface plasmon resonance (SPR). In some embodiments, the antibody or antigen-binding fragment thereof as described herein or ADC derived therefrom binds to human CD79b, monkey CD79b, dog CD79b, and/or mouse CD79b. In some embodiments, the antibody does not bind to human CD79b, monkey CD79b, dog CD79b, and/or mouse CD79b.

Furthermore, an alternatively spliced isoform has been described for CD79b in Chronic lymphocyte Leukemia. This isoform (also referred to as the “short isoform”) encodes 125 amino acids (SEQ ID NO: 67) and differs from the 229 amino acids of the wild type (also referred to as the “long isoform”) (SEQ ID NO: 68) through a deletion of exon 3 which essentially encodes most of the extracellular domain. In some embodiments, the anti-CD79b antibodies described herein can bind to both isoforms of CD79b. In some embodiments, the anti-CD79b antibodies bind to a CD79b isoform (e.g., SEQ ID NO: 67). In some embodiments, the anti-CD79b antibodies do not bind to a CD79b isoform (e.g., SEQ ID NO: 67). In some embodiments, the anti-CD79b antibodies bind to the wildtype CD79b (e.g., SEQ ID NO: 68). In some embodiments, the antibodies as described herein bind to the extracellular domain of CD79b.

In some embodiments, the anti-CD79b antibodies bind to a CD79b variant (e.g., SEQ ID NO: 2). In some embodiments, the anti-CD79b antibodies do not bind to a CD79b variant (e.g., SEQ ID NO: 2). In some embodiments, the anti-CD79b antibodies bind to an epitope located within amino acids 1-13 of the ECD of CD79b (e.g., SEQ ID NO: 1).

In some embodiments, the antibody or antigen-binding fragment thereof as described herein or ADC derived therefrom is added to Ramos cells to test the internalization rate. In some embodiments, at various time points (e.g., 1 h, 3 h, or 6 h), the antibody or antigen-binding fragment thereof as described herein or ADC derived therefrom has an internalization rate of above 5%, above 10%, above 15%, above 20%, above 25%, above 30%, above 35%, above 40%, above 45%, above 50%, above 55%, above 60%, above 65%, above 70%, above 75%, above 80%, above 85%, above 90%, above 91%, above 92%, above 93%, above 94%, above 95%, above 96%, above 97%, or above 98%. In some embodiments, the antibody or antigen-binding fragment thereof as described herein or ADC derived therefrom has a slower internalization rate comparing to polatuzumab.

In some embodiments, thermal stabilities are determined. The antibody or antigen-binding fragment thereof as described herein or ADC derived therefrom can have a Tm greater than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C. In some embodiments, Tm is less than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C.

As IgG can be described as a multi-domain protein, the melting curve sometimes shows two transitions, with a first denaturation temperature, Tm 1, and a second denaturation temperature Tm 2. The presence of these two peaks often indicate the denaturation of the Fc domains (Tm 1) and Fab domains (Tm 2), respectively. Thus, in some embodiments, the antibodies or antigen binding fragments as described herein has a Tm 1 greater than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C. In some embodiments, the antibodies or antigen binding fragments as described herein has a Tm 2 greater than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C.

In some embodiments, Tm, Tm 1, Tm 2 are less than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C.

In some embodiments, the antibody or antigen-binding fragment thereof as described herein or ADC derived therefrom can bind to human or monkey CD79b, as measured by ELISA. In some embodiments, the antibodies or antigen binding fragments as described herein or ADC derived therefrom can bind to human or monkey CD79b with a IC50 of less than 200 nM, less than 100 nM, less than 50 nM, less than 10 nM, less than 5 nM, less than 1 nM, less than 0.75 nM, less than 0.5 nM, or less than 0.25 nM.

In some embodiments, the antibody or antigen-binding fragment thereof as described herein or ADC derived therefrom can bind to the same epitope of CD79b. In some embodiments, the antibodies or antigen-binding fragments thereof as described herein or ADC derived therefrom can bind to different epitopes of CD79b.

In some embodiments, the antibody or antigen-binding fragment thereof as described herein or ADC derived therefrom can bind to malignant B cell lines with both high and low CD79b antigen densities (e.g., BJAB, Ramos, Daudi, SU-DHL-4 and Nalm-6), as determined by flow cytometry. In some embodiments, the EC50 is less than 200 nM, less than 100 nM, less than 50 nM, less than 10 nM, less than 5 nM, less than 1 nM, less than 0.75 nM, less than 0.5 nM, or less than 0.25 nM.

In some embodiments, the antibody or antigen-binding fragment thereof as described herein or ADC derived therefrom can bind to human B cells with higher cell surface binding affinities than Polatuzumab. In some embodiments, the EC50 is less than 200 nM, less than 100 nM, less than 50 nM, less than 10 nM, less than 5 nM, less than 1 nM, less than 0.75 nM, less than 0.5 nM, or less than 0.25 nM.

In some embodiments, the antibody or antigen-binding fragment thereof as described herein or ADC derived therefrom can bind to cell surface CD79b on Chronic Lymphocytic Leukemia (CLL) patient's B lymphocytes. In some embodiments, the binding is more potent than polatuzumab.

In some embodiments, the ADC described herein has an average drug-to-antibody ratio (DAR) of higher than 3, higher than 3.2, higher than 3.4, higher than 3.6, higher than 3.8, higher than 4, higher than 4.2, higher than 4.4, or higher than 4.6, as determined by HPLC. In some embodiments, the ADC described herein has an average DAR of lower than 3, lower than 3.2, lower than 3.4, lower than 3.6, lower than 3.8, lower than 4, lower than 4.2, lower than 4.4 or lower than 4.6, as determined by HPLC. In some embodiments, the DAR is about 4.

In some embodiments, the antibody or antigen-binding fragment thereof as described herein or ADC derived therefrom has a tumor growth inhibition percentage (TGI %) that is greater than 10%, 20%, 30%, 40%, 50%, 60%, 70%, 80%, 90%, 100%, 110%, 120%, 130%, 140%, 150%, 160%, 170%, 180%, 190%, or 200%. In some embodiments, the antibody or antigen-binding fragment thereof as described herein or ADC derived therefrom has a tumor growth inhibition percentage that is less than 60%, 70%, 80%, 90%, 100%, 110%, 120%, 130%, 140%, 150%, 160%, 170%, 180%, 190%, or 200%. The TGI % can be determined, e.g., at 3, 4, 5, 6, 7, 8, 9, 10, 11, 12, 13, 14, 15, 16, 17, 18, 19, 20, 21, 22, 23, 24, 25, 26, 27, 28, 29, or 30 days after the treatment starts, or 1, 2, 3, 4, 5, 6, 7, 8, 9, 10, 11, or 12 months after the treatment starts. As used herein, the tumor growth inhibition percentage (TGI %) is calculated using the following formula:

TGI (%)=[1−(Ti−T0)/(Vi−V0)]×100

Ti is the average tumor volume in the treatment group on day i. T0 is the average tumor volume in the treatment group on day zero. Vi is the average tumor volume in the control group on day i. V0 is the average tumor volume in the control group on day zero.

In some embodiments, the antibody or antigen-binding fragment thereof as described herein or ADC derived therefrom can bind to tumor cells that express CD79b. In some embodiments, the antibodies or antigen binding fragments as described herein or ADC derived therefrom can induce complement-dependent cytotoxicity (CDC) and/or antibody dependent cellular cytoxicity (ADCC), and kill the tumor cell.

In some embodiments, the antibody or antigen-binding fragment thereof as described herein or ADC derived therefrom has a functional Fc region. In some embodiments, effector function of a functional Fc region is antibody-dependent cell-mediated cytotoxicity (ADCC). In some embodiments, effector function of a functional Fc region is phagocytosis. In some embodiments, effector function of a functional Fc region is ADCC and phagocytosis.

In some embodiments, the Fc region is human IgG1, human IgG2, human IgG3, or human IgG4. In some embodiments, the antibody is a humanized IgG1 antibody, optionally with SI mutations, LALA mutations, N297A mutation, YTE mutations, and/or FLAA mutations. In some embodiments, the antibody is a humanized IgG4 antibody, optionally with SI mutations, LALA mutations, N297A mutation, YTE mutations, and/or FLAA mutations.

In some embodiments, the antibodies or antigen binding fragments as described herein or ADC derived therefrom do not have a functional Fe region. For example, the antibodies or antigen binding fragments are Fab, Fab′, F(ab′)₂, and Fv fragments. In some embodiments, the Fc region has LALA mutations (L234A and L235A mutations according to EU numbering), or LALA-PG mutations (L234A, L235A, P329G mutations according to EU numbering). In some embodiments, the Fc region has FLAA mutations (F234A and L235A according to EU numbering). In some embodiments, the Fc has SI mutations (S239D and 1332E mutations according to EU numbering). In some embodiments, the Fc has N297A mutation according to EU numbering. In some embodiments, the Fc has YTE mutations (M252Y, S254T and T256E according to EU numbering).

### Methods of Making Anti-CD79b Antibodies

An isolated fragment of human CD79b (e.g., CD79b extracellular domain) can be used as an immunogen to generate antibodies using standard techniques for polyclonal and monoclonal antibody preparation. Polyclonal antibodies can be raised in animals by multiple injections (e.g., subcutaneous or intraperitoneal injections) of an antigenic peptide or protein. In some embodiments, the antigenic peptide or protein is injected with at least one adjuvant. In some embodiments, the antigenic peptide or protein can be conjugated to an agent that is immunogenic in the species to be immunized. Animals can be injected with the antigenic peptide or protein more than one time (e.g., twice, three times, or four times).

The full-length polypeptide or protein (or the extracellular region thereof) can be used or, alternatively, antigenic peptide fragments thereof can be used as immunogens. The antigenic peptide of a protein comprises at least 8 (e.g., at least 10, 15, 20, or 30) amino acid residues of the amino acid sequence of CD79b and encompasses an epitope of the protein such that an antibody raised against the peptide forms a specific immune complex with the protein. As described above, the full length sequence of human CD79b is known in the art. In some embodiments, an Fc-tagged or His-tagged human CD79b protein is used as the immunogen. In some embodiments, the extracellular domain (ECD) of human CD79b is used as the immunogen.

An immunogen typically is used to prepare antibodies by immunizing a suitable subject (e.g., human or transgenic animal expressing at least one human immunoglobulin locus). An appropriate immunogenic preparation can contain, for example, a recombinantly-expressed or a chemically-synthesized polypeptide (e.g., a fragment of human CD79b). The preparation can further include an adjuvant, such as Freund's complete or incomplete adjuvant, or a similar immunostimulatory agent.

Polyclonal antibodies can be prepared as described above by immunizing a suitable subject with a CD79b polypeptide, or an antigenic peptide thereof (e.g., part of CD79b) as an immunogen. The antibody titer in the immunized subject can be monitored over time by standard techniques, such as with an enzyme-linked immunosorbent assay (ELISA) using the immobilized CD79b polypeptide or peptide. If desired, the antibody molecules can be isolated from the mammal (e.g., from the blood) and further purified by well-known techniques, such as protein A of protein G chromatography to obtain the IgG fraction. At an appropriate time after immunization, e.g., when the specific antibody titers are highest, antibody-producing cells can be obtained from the subject and used to prepare monoclonal antibodies by standard techniques, such as the hybridoma technique originally described by Kohler et al. (Nature 256:495-497, 1975), the human B cell hybridoma technique (Kozbor et al., Immunol. Today 4:72, 1983), the EBV-hybridoma technique (Cole et al., Monoclonal Antibodies and Cancer Therapy, Alan R. Liss, Inc., pp. 77-96, 1985), or trioma techniques. The technology for producing hybridomas is well known (see, generally, Current Protocols in Immunology, 1994, Coligan et al. (Eds.), John Wiley & Sons, Inc., New York, NY). Hybridoma cells producing a monoclonal antibody are detected by screening the hybridoma culture supernatants for antibodies that bind the polypeptide or epitope of interest, e.g., using a standard ELISA assay.

Variants of the antibodies or antigen-binding fragments described herein can be prepared by introducing appropriate nucleotide changes into the DNA encoding a human, humanized, or chimeric antibody, or antigen-binding fragment thereof described herein, or by peptide synthesis. Such variants include, for example, deletions, insertions, or substitutions of residues within the amino acids sequences that make-up the antigen-binding site of the antibody or an antigen-binding domain. In a population of such variants, some antibodies or antigen-binding fragments will have increased affinity for the target protein, e.g., CD79b. Any combination of deletions, insertions, and/or combinations can be made to arrive at an antibody or antigen-binding fragment thereof that has increased binding affinity for the target. The amino acid changes introduced into the antibody or antigen-binding fragment can also alter or introduce new post-translational modifications into the antibody or antigen-binding fragment, such as changing (e.g., increasing or decreasing) the number of glycosylation sites, changing the type of glycosylation site (e.g., changing the amino acid sequence such that a different sugar is attached by enzymes present in a cell), or introducing new glycosylation sites.

Antibodies disclosed herein can be derived from any species of animal, including mammals. Non-limiting examples of native antibodies include antibodies derived from humans, primates (e.g., monkeys and apes), rabbits, cows, pigs, horses, sheep, camelids (e.g., camels and llamas), chicken, goats, and rodents (e.g., rats, mice, hamsters and rabbits), including transgenic animals genetically engineered to produce human antibodies.

Human and humanized antibodies include antibodies having variable and constant regions derived from (or having the same amino acid sequence as those derived from) human germline immunoglobulin sequences. Human antibodies may include amino acid residues not encoded by human germline immunoglobulin sequences (e.g., mutations introduced by random or site-specific mutagenesis in vitro or by somatic mutation in vivo), for example in the CDRs.

A humanized antibody, typically has a human framework (FR) grafted with non-human CDRs. Thus, a humanized antibody has one or more amino acid sequence introduced into it from a source which is non-human. These non-human amino acid residues are often referred to as “import” residues, which are typically taken from an “import” variable domain. Humanization can be essentially performed by e.g., substituting rodent CDRs or CDR sequences for the corresponding sequences of a human antibody. These methods are described in e.g., Jones et al., Nature, 321:522-525 (1986); Riechmann et al., Nature, 332:323-327 (1988); Verhoeyen et al., Science, 239:1534-1536 (1988); each of which is incorporated by reference herein in its entirety. Accordingly, “humanized” antibodies are chimeric antibodies wherein substantially less than an intact human V domain has been substituted by the corresponding sequence from a non-human species. In practice, humanized antibodies are typically non-human antibodies in which some CDR residues and some FR residues are substituted by residues from analogous sites in human antibodies.

The choice of human VH and VL domains to be used in making the humanized antibodies is very important for reducing immunogenicity. According to the so-called “best-fit” method, the sequence of the V domain of a non-human antibody is screened against the entire library of known human-domain sequences. The human sequence which is closest to that of the non-human animal is then accepted as the human FR for the humanized antibody (Sims et al., J. Immunol., 151:2296 (1993); Chothia et al., J. Mol. Biol., 196:901 (1987)).

It is further important that antibodies be humanized with retention of high specificity and affinity for the antigen and other favorable biological properties. To achieve this goal, humanized antibodies can be prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved.

Ordinarily, amino acid sequence variants of the human, humanized, or chimeric anti-CD79b antibody will contain an amino acid sequence having at least 75%, 80%, 85%, 90%, 95%, 96%, 97%, 98%, or 99% percent identity with a sequence present in the light or heavy chain of the original antibody.

Identity or homology with respect to an original sequence is usually the percentage of amino acid residues present within the candidate sequence that are identical with a sequence present within the human, humanized, or chimeric anti-CD79b antibody or fragment, after aligning the sequences and introducing gaps, if necessary, to achieve the maximum percent sequence identity, and not considering any conservative substitutions as part of the sequence identity.

Additional modifications to the anti-CD79b antibodies or antigen-binding fragments can be made. For example, a cysteine residue(s) can be introduced into the Fc region, thereby allowing interchain disulfide bond formation in this region. The homodimeric antibody thus generated may have any increased half-life in vitro and/or in vivo. Homodimeric antibodies with increased half-life in vitro and/or in vivo can also be prepared using heterobifunctional cross-linkers as described, for example, in Wolff et al. (Cancer Res. 53:2560-2565, 1993). Alternatively, an antibody can be engineered which has dual Fc regions (see, for example, Stevenson et al., Anti-Cancer Drug Design 3:219-230, 1989).

In some embodiments, a covalent modification can be made to the anti-CD79b antibody or antigen-binding fragment thereof. These covalent modifications can be made by chemical or enzymatic synthesis, or by enzymatic or chemical cleavage. Other types of covalent modifications of the antibody or antibody fragment are introduced into the molecule by reacting targeted amino acid residues of the antibody or fragment with an organic derivatization agent that is capable of reacting with selected side chains or the N- or C-terminal residues.

In some embodiments, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues; or position 314 in Kabat numbering); however, Asn297 may also be located about 3 amino acids upstream or downstream of position 297, i.e., between positions 294 and 300, due to minor sequence variations in antibodies. Such fucosylation variants may have improved ADCC function. In some embodiments, to reduce glycan heterogeneity, the Fc region of the antibody can be further engineered to replace the Asparagine at position 297 with Alanine (N297A).

In some embodiments, to facilitate production efficiency by avoiding Fab-arm exchange, the Fc region of the antibodies was further engineered to replace the serine at position 228 (EU numbering) of IgG4 with proline (S228P). A detailed description regarding S228 mutation is described, e.g., in Silva et al. “The S228P mutation prevents in vivo and in vitro IgG4 Fab-arm exchange as demonstrated using a combination of novel quantitative immunoassays and physiological matrix preparation.” Journal of Biological Chemistry 290.9 (2015): 5462-5469, which is incorporated by reference in its entirety.

### Recombinant Vectors

The present disclosure also provides recombinant vectors (e.g., an expression vectors) that include an isolated polynucleotide disclosed herein (e.g., a polynucleotide that encodes a polypeptide disclosed herein), host cells into which are introduced the recombinant vectors (i.e., such that the host cells contain the polynucleotide and/or a vector comprising the polynucleotide), and the production of recombinant antibody polypeptides or fragments thereof by recombinant techniques.

As used herein, a “vector” is any construct capable of delivering one or more polynucleotide(s) of interest to a host cell when the vector is introduced to the host cell. An “expression vector” is capable of delivering and expressing the one or more polynucleotide(s) of interest as an encoded polypeptide in a host cell into which the expression vector has been introduced. Thus, in an expression vector, the polynucleotide of interest is positioned for expression in the vector by being operably linked with regulatory elements such as a promoter, enhancer, and/or a poly-A tail, either within the vector or in the genome of the host cell at or near or flanking the integration site of the polynucleotide of interest such that the polynucleotide of interest will be translated in the host cell introduced with the expression vector.

A vector can be introduced into the host cell by methods known in the art, e.g., electroporation, chemical transfection (e.g., DEAE-dextran), transformation, transfection, and infection and/or transduction (e.g., with recombinant virus). Thus, non-limiting examples of vectors include viral vectors (which can be used to generate recombinant virus), naked DNA or RNA, plasmids, cosmids, phage vectors, and DNA or RNA expression vectors associated with cationic condensing agents.

In some implementations, a polynucleotide disclosed herein (e.g., a polynucleotide that encodes a polypeptide disclosed herein) is introduced using a viral expression system (e.g., vaccinia or other pox virus, retrovirus, or adenovirus), which may involve the use of a non-pathogenic (defective), replication competent virus, or may use a replication defective virus. In the latter case, viral propagation generally will occur only in complementing virus packaging cells. Suitable systems are disclosed, for example, in Fisher-Hoch et al., 1989, Proc. Natl. Acad. Sci. USA 86:317-321; Flexner et al., 1989, Ann. N.Y. Acad Sci. 569:86-103, Flexner et al., 1990, Vaccine, 8:17-21; U.S. Pat. Nos. 4,603,112, 4,769,330, and 5,017,487; WO 89/01973; U.S. Pat. No. 4,777,127; GB 2,200,651; EP 0,345,242; WO 91/02805; Berkner-Biotechniques, 6:616-627, 1988; Rosenfeld et al., 1991, Science, 252:431-434; Kolls et al., 1994, Proc. Natl. Acad. Sci. USA, 91:215-219; Kass-Eisler et al., 1993, Proc. Natl. Acad. Sci. USA, 90:11498-11502; Guzman et al., 1993, Circulation, 88:2838-2848; and Guzman et al., 1993, Cir. Res., 73:1202-1207. Techniques for incorporating DNA into such expression systems are well known to those of ordinary skill in the art. The DNA may also be “naked,” as described, for example, in Ulmer et al., 1993, Science, 259:1745-1749, and Cohen, 1993, Science, 259:1691-1692. The uptake of naked DNA may be increased by coating the DNA onto biodegradable beads that are efficiently transported into the cells.

For expression, the DNA insert comprising an antibody-encoding or polypeptide-encoding polynucleotide disclosed herein can be operatively linked to an appropriate promoter (e.g., a heterologous promoter), such as the phage lambda PL promoter, the E. coli lac, trp and tac promoters, the SV40 early and late promoters and promoters of retroviral LTRs, to name a few. Other suitable promoters are known to the skilled artisan. The expression constructs can further contain sites for transcription initiation, termination and, in the transcribed region, a ribosome binding site for translation. The coding portion of the mature transcripts expressed by the constructs may include a translation initiating at the beginning and a termination codon (UAA, UGA, or UAG) appropriately positioned at the end of the polypeptide to be translated.

As indicated, the expression vectors can include at least one selectable marker. Such markers include dihydrofolate reductase or neomycin resistance for eukaryotic cell culture and tetracycline or ampicillin resistance genes for culturing in E. coli and other bacteria. Representative examples of appropriate hosts include, but are not limited to, bacterial cells, such as E. coli, Streptomyces, and Salmonella typhimurium cells; fungal cells, such as yeast cells; insect cells such as Drosophila S2 and Spodoptera Sf9 cells; animal cells such as CHO, COS, Bowes melanoma, and HK 293 cells; and plant cells. Appropriate culture mediums and conditions for the host cells described herein are known in the art.

Non-limiting vectors for use in bacteria include pQE70, pQE60 and pQE-9, available from Qiagen; pBS vectors, Phagescript vectors, Bluescript vectors, pNH8A, pNH16a, pNH18A, pNH46A, available from Stratagene; and ptrc99a, pKK223-3, pKK233-3, pDR540, pRIT5 available from Pharmacia. Non-limiting eukaryotic vectors include pWLNEO, pSV2CAT, pOG44, pXT1 and pSG available from Stratagene; and pSVK3, pBPV, pMSG and pSVL available from Pharmacia. Other suitable vectors will be readily apparent to the skilled artisan.

Non-limiting bacterial promoters suitable for use include the E. coli lacI and lacZ promoters, the T3 and T7 promoters, the gpt promoter, the lambda PR and PL promoters and the trp promoter. Suitable eukaryotic promoters include the CMV immediate early promoter, the HSV thymidine kinase promoter, the early and late SV40 promoters, the promoters of retroviral LTRs, such as those of the Rous sarcoma virus (RSV), and metallothionein promoters, such as the mouse metallothionein-I promoter.

In the yeast Saccharomyces cerevisiae, a number of vectors containing constitutive or inducible promoters such as alpha factor, alcohol oxidase, and PGH may be used. For reviews, see Ausubel et al. (1989) Current Protocols in Molecular Biology, John Wiley & Sons, New York, N.Y, and Grant et al., Methods Enzymol., 153: 516-544 (1997).

Introduction of the construct into the host cell can be effected by calcium phosphate transfection, DEAE-dextran mediated transfection, cationic lipid-mediated transfection, electroporation, transduction, infection or other methods. Such methods are described in many standard laboratory manuals, such as Davis et al., Basic Methods In Molecular Biology (1986), which is incorporated herein by reference in its entirety.

Transcription of DNA encoding an antibody of the present disclosure by higher eukaryotes may be increased by inserting an enhancer sequence into the vector. Enhancers are cis-acting elements of DNA, usually about from 10 to 300 bp that act to increase transcriptional activity of a promoter in a given host cell-type. Examples of enhancers include the SV40 enhancer, which is located on the late side of the replication origin at base pairs 100 to 270, the cytomegalovirus early promoter enhancer, the polyoma enhancer on the late side of the replication origin, and adenovirus enhancers.

For secretion of the translated protein into the lumen of the endoplasmic reticulum, into the periplasmic space or into the extracellular environment, appropriate secretion signals may be incorporated into the expressed polypeptide. The signals may be endogenous to the polypeptide or they may be heterologous signals.

The polypeptide (e.g., antibody) can be expressed in a modified form, such as a fusion protein (e.g., a GST-fusion) or with a histidine-tag, and may include not only secretion signals, but also additional heterologous functional regions. For instance, a region of additional amino acids, particularly charged amino acids, may be added to the N-terminus of the polypeptide to improve stability and persistence in the host cell, during purification, or during subsequent handling and storage. Also, peptide moieties can be added to the polypeptide to facilitate purification. Such regions can be removed prior to final preparation of the polypeptide. The addition of peptide moieties to polypeptides to engender secretion or excretion, to improve stability and to facilitate purification, among others, are familiar and routine techniques in the art.

### Methods of Treatment

The antibodies or antigen-binding fragments thereof of the present disclosure can be used for various therapeutic purposes.

In one aspect, the disclosure provides methods for treating a cancer in a subject, methods of reducing the rate of the increase of volume of a tumor in a subject over time, methods of reducing the risk of developing a metastasis, or methods of reducing the risk of developing an additional metastasis in a subject. In some embodiments, the treatment can halt, slow, retard, or inhibit progression of a cancer. In some embodiments, the treatment can result in the reduction of in the number, severity, and/or duration of one or more symptoms of the cancer in a subject.

In one aspect, the disclosure features methods that include administering a therapeutically effective amount of an antibody or antigen-binding fragment thereof disclosed herein to a subject in need thereof (e.g., a subject having, or identified or diagnosed as having, a cancer), e.g., breast cancer (e.g., triple-negative breast cancer), carcinoid cancer, cervical cancer, endometrial cancer, glioma, head and neck cancer, liver cancer, lung cancer, small cell lung cancer, lymphoma, melanoma, ovarian cancer, pancreatic cancer, prostate cancer, renal cancer, colorectal cancer, gastric cancer, testicular cancer, thyroid cancer, bladder cancer, urethral cancer, or hematologic malignancy. In some embodiments, the cancer is unresectable melanoma or metastatic melanoma, non-small cell lung carcinoma (NSCLC), small cell lung cancer (SCLC), bladder cancer, or metastatic hormone-refractory prostate cancer. In some embodiments, the cancer is NSCLC, ovarian cancer, melanoma, colorectal cancer, breast cancer, a hematological malignancy, head and neck cancer, gastrointestinal cancer, bladder cancer, or bone cancer. In some embodiments, the subject has Hodgkin's lymphoma. In some embodiments, the subject has triple-negative breast cancer (TNBC), gastric cancer, urothelial cancer, Merkel-cell carcinoma, or head and neck cancer. In some embodiments, the cancer is melanoma, pancreatic carcinoma, mesothelioma, hematological malignancies, especially Non-Hodgkin's lymphoma, lymphoma, chronic lymphocytic leukemia, or advanced solid tumors. In some embodiments, the cancer is Lymphoma, breast cancer, stomach cancer, pancreatic cancer, prostate cancer, cervical cancer, endometrial cancer, ovarian cancer or urothelial cancer.

In one aspect, the disclosure provides methods for treating a subject having autoimmune disease, the method comprising administering a therapeutically effective amount of a composition comprising the antibody or antigen-binding fragment thereof, the CAR, or the antibody-drug conjugate described herein to the subject.

In some embodiments, the autoimmune disease is selected from rheumatoid arthritis, psoriasis, multiple sclerosis, immune thrombocytopenic purpura, myasthenia gravis, neuromyelitis optica, IgG4-related diseases, systemic Lupus Erythematosus, lupus nephritis, giant cell arteritis, takayasu disease, cold agglutinin disease, warm autoimmune hemolytic anemia, and anti-neutrophil cytoplasmic antibody (ANCA) associated vasculitides including for example, tranulomatosis with polyangiitis (GPA) (Wegener's Granulomatosis) or Microscopic Polyangiitis (MPA).

In some embodiments, the autoimmune disease is multiple sclerosis, systemic lupus erythematosus or rheumatoid arthritis.

In some embodiments, the compositions and methods disclosed herein can be used for treatment of patients at risk for a cancer. Patients with cancer can be identified with various methods known in the art.

In one aspect, the disclosure provides methods for treating, preventing, or reducing the risk of developing disorders associated with an abnormal or unwanted immune response, e.g., an autoimmune disorder. These autoimmune disorders include, but are not limited to, Alopecia areata, lupus, ankylosing spondylitis, Meniere's disease, antiphospholipid syndrome, mixed connective tissue disease, autoimmune Addison's disease, multiple sclerosis, autoimmune hemolytic anemia, myasthenia gravis, autoimmune hepatitis, pemphigus vulgaris, Behcet's disease, pernicious anemia, bullous pemphigoid, polyarthritis nodosa, cardiomyopathy, polychondritis, celiac sprue-dermatitis, polyglandular syndromes, chronic fatigue syndrome (CFIDS), polymyalgia rheumatica, chronic inflammatory demyelinating, polymyositis and dermatomyositis, chronic inflammatory polyneuropathy, primary agammaglobulinemia, Churg-Strauss syndrome, primary biliary cirrhosis, cicatricial pemphigoid, psoriasis, CREST syndrome, Raynaud's phenomenon, cold agglutinin disease, Reiter's syndrome, Crohn's disease, Rheumatic fever, discoid lupus, rheumatoid arthritis, Cryoglobulinemia sarcoidosis, fibromyalgia, scleroderma, Grave's disease, Sjögren's syndrome, Guillain-Barre, stiff-man syndrome, Hashimoto's thyroiditis, Takayasu arteritis, idiopathic pulmonary fibrosis, temporal arteritis/giant cell arteritis, idiopathic thrombocytopenia purpura (ITP), ulcerative colitis, IgA nephropathy, uveitis, diabetes (e.g., Type I), vasculitis, lichen planus, and vitiligo. The anti-CD79b antibodies or antigen-binding fragments thereof can also be administered to a subject to treat, prevent, or reduce the risk of developing disorders associated with an abnormal or unwanted immune response associated with cell, tissue or organ transplantation, e.g., renal, hepatic, and cardiac transplantation, e.g., graft versus host disease (GVHD), or to prevent allograft rejection. In some embodiments, the subject has dermatological disorders, liver disease (e.g., cirrhosis), Hidradenitis, experimental autoimmune encephalomyelitis. In some embodiments, the subject has renal disease, lupus, Sjogren's syndrome, ulcerative colitics, psoriasis, Hidradenitis suppurativa, Immune Thrombocytopenia (ITP), or other inflammatory arthritis. In some embodiments, the subject has multiple sclerosis or myasthenia gravis. In some embodiments, the subject has Crohn's disease, ulcerative colitis or type 1 diabetes. In some embodiments, the subject has autoimmune thyroid disease, Grave's disease, multiple sclerosis, psoriasis, inflammatory bowel disease (e.g., Crohn's Disease (CD) and ulcerative colitis), rheumatoid arthritis, Sjögren's syndrome, autoimmune nephritis, or systemic lupus erythematosus. In some embodiments, the methods involve administering to the subject an effective amount of a composition comprising the antibody or antigen-binding fragment thereof as described herein.

As used herein, by an “effective amount” is meant an amount or dosage sufficient to effect beneficial or desired results including halting, slowing, retarding, or inhibiting progression of a disease, e.g., an autoimmune disease or a cancer. An effective amount will vary depending upon, e.g., an age and a body weight of a subject to which the antibody, antigen binding fragment, antibody-encoding polynucleotide, vector comprising the polynucleotide, and/or compositions thereof is to be administered, a severity of symptoms and a route of administration, and thus administration can be determined on an individual basis.

An effective amount can be administered in one or more administrations. By way of example, an effective amount of an antibody or an antigen binding fragment is an amount sufficient to ameliorate, stop, stabilize, reverse, inhibit, slow and/or delay progression of an autoimmune disease or a cancer in a patient or is an amount sufficient to ameliorate, stop, stabilize, reverse, slow and/or delay proliferation of a cell (e.g., a biopsied cell, any of the cancer cells described herein, or cell line (e.g., a cancer cell line)) in vitro. As is understood in the art, an effective amount of an antibody or antigen binding fragment may vary, depending on, inter alia, patient history as well as other factors such as the type (and/or dosage) of antibody used.

Effective amounts and schedules for administering the antibodies, antibody-encoding polynucleotides, and/or compositions disclosed herein may be determined empirically, and making such determinations is within the skill in the art. Those skilled in the art will understand that the dosage that must be administered will vary depending on, for example, the mammal that will receive the antibodies, antibody-encoding polynucleotides, and/or compositions disclosed herein, the route of administration, the particular type of antibodies, antibody-encoding polynucleotides, antigen binding fragments, and/or compositions disclosed herein used and other drugs being administered to the mammal. Guidance in selecting appropriate doses for antibody or antigen binding fragment can be found in the literature on therapeutic uses of antibodies and antigen binding fragments, e.g., Handbook of Monoclonal Antibodies, Ferrone et al., eds., Noges Publications, Park Ridge, N.J., 1985, ch. 22 and pp. 303-357; Smith et al., Antibodies in Human Diagnosis and Therapy, Haber et al., eds., Raven Press, New York, 1977, pp. 365-389.

A typical daily dosage of an effective amount of an antibody is 0.01 mg/kg to 100 mg/kg. In some embodiments, the dosage can be less than 100 mg/kg, 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, or 0.1 mg/kg. In some embodiments, the dosage can be greater than 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, 0.1 mg/kg, 0.05 mg/kg, or 0.01 mg/kg. In some embodiments, the dosage is about 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.9 mg/kg, 0.8 mg/kg, 0.7 mg/kg, 0.6 mg/kg, 0.5 mg/kg, 0.4 mg/kg, 0.3 mg/kg, 0.2 mg/kg, or 0.1 mg/kg.

In any of the methods described herein, the at least one antibody, antigen-binding fragment thereof, or pharmaceutical composition (e.g., any of the antibodies, antigen-binding fragments, or pharmaceutical compositions described herein) and, optionally, at least one additional therapeutic agent can be administered to the subject at least once a week (e.g., once a week, twice a week, three times a week, four times a week, once a day, twice a day, or three times a day). In some embodiments, at least two different antibodies and/or antigen-binding fragments are administered in the same composition (e.g., a liquid composition). In some embodiments, at least one antibody or antigen-binding fragment and at least one additional therapeutic agent are administered in the same composition (e.g., a liquid composition). In some embodiments, the at least one antibody or antigen-binding fragment and the at least one additional therapeutic agent are administered in two different compositions (e.g., a liquid composition containing at least one antibody or antigen-binding fragment and a solid oral composition containing at least one additional therapeutic agent). In some embodiments, the at least one additional therapeutic agent is administered as a pill, tablet, or capsule. In some embodiments, the at least one additional therapeutic agent is administered in a sustained-release oral formulation.

In some embodiments, the one or more additional therapeutic agents can be administered to the subject prior to, or after administering the at least one antibody, antigen-binding antibody fragment, or pharmaceutical composition (e.g., any of the antibodies, antigen-binding antibody fragments, or pharmaceutical compositions described herein). In some embodiments, the one or more additional therapeutic agents and the at least one antibody, antigen-binding antibody fragment, or pharmaceutical composition (e.g., any of the antibodies, antigen-binding antibody fragments, or pharmaceutical compositions described herein) are administered to the subject such that there is an overlap in the bioactive period of the one or more additional therapeutic agents and the at least one antibody or antigen-binding fragment (e.g., any of the antibodies or antigen-binding fragments described herein) in the subject.

In some embodiments, the subject can be administered the at least one antibody, antigen-binding antibody fragment, or pharmaceutical composition (e.g., any of the antibodies, antigen-binding antibody fragments, or pharmaceutical compositions described herein) over an extended period of time (e.g., over a period of at least 1 week, 2 weeks, 3 weeks, 1 month, 2 months, 3 months, 4 months, 5 months, 6 months, 7 months, 8 months, 9 months, 10 months, 11 months, 12 months, 1 year, 2 years, 3 years, 4 years, or 5 years). A skilled medical professional may determine the length of the treatment period using any of the methods described herein for diagnosing or following the effectiveness of treatment (e.g., the observation of at least one symptom of cancer). As described herein, a skilled medical professional can also change the identity and number (e.g., increase or decrease) of antibodies or antigen-binding antibody fragments (and/or one or more additional therapeutic agents) administered to the subject and can also adjust (e.g., increase or decrease) the dosage or frequency of administration of at least one antibody or antigen-binding antibody fragment (and/or one or more additional therapeutic agents) to the subject based on an assessment of the effectiveness of the treatment (e.g., using any of the methods described herein and known in the art).

In some embodiments, one or more additional therapeutic agents can be administered to the subject. The additional therapeutic agent can comprise one or more inhibitors selected from the group consisting of an inhibitor of B-Raf, an EGFR inhibitor, an inhibitor of a MEK, an inhibitor of ERK, an inhibitor of K-Ras, an inhibitor of c-Met, an inhibitor of anaplastic lymphoma kinase (ALK), an inhibitor of a phosphatidylinositol 3-kinase (PI3K), an inhibitor of an Akt, an inhibitor of mTOR, a dual PI3K/mTOR inhibitor, an inhibitor of Bruton's tyrosine kinase (BTK), and an inhibitor of Isocitrate dehydrogenase 1 (IDH1) and/or Isocitrate dehydrogenase 2 (IDH2). In some embodiments, the additional therapeutic agent is an inhibitor of indoleamine 2,3-dioxygenase-1) (IDO1) (e.g., epacadostat).

In some embodiments, the additional therapeutic agent can comprise one or more inhibitors selected from the group consisting of an inhibitor of PD-1, an inhibitor of LSD1, an inhibitor of MDM2, an inhibitor of BCL2, an inhibitor of CHK1, an inhibitor of activated hedgehog signaling pathway, and an agent that selectively degrades the estrogen receptor.

In some embodiments, the additional therapeutic agent can comprise one or more therapeutic agents selected from the group consisting of Trabectedin, nab-paclitaxel, Trebananib, Pazopanib, Cediranib, Palbociclib, everolimus, fluoropyrimidine, IFL, regorafenib, Reolysin, Alimta, Zykadia, Sutent, temsirolimus, axitinib, everolimus, sorafenib, Votrient, Pazopanib, IMA-901, AGS-003, cabozantinib, Vinflunine, an Hsp90 inhibitor, Ad-GM-CSF, Temazolomide, IL-2, IFNa, vinblastine, Thalomid, dacarbazine, cyclophosphamide, lenalidomide, azacytidine, lenalidomide, bortezomid, amrubicine, carfilzomib, pralatrexate, and enzastaurin.

In some embodiments, the additional therapeutic agent can comprise one or more therapeutic agents selected from the group consisting of an adjuvant, a TLR agonist, tumor necrosis factor (TNF) alpha, IL-1, HMGB1, an IL-10 antagonist, an IL-4 antagonist, an IL-13 antagonist, an IL-17 antagonist, an HVEM antagonist, an ICOS agonist, a treatment targeting CX3CL1, a treatment targeting CXCL9, a treatment targeting CXCL10, a treatment targeting CCL5, an LFA-1 agonist, an ICAM1 agonist, and a PD-1 agonist.

In some embodiments, carboplatin, nab-paclitaxel, paclitaxel, cisplatin, pemetrexed, gemcitabine, FOLFOX, or FOLFIRI are administered to the subject.

In some embodiments, the additional therapeutic agent is an anti-OX40 antibody, an anti-PD-1 antibody, an anti-PD-L2 antibody, an anti-LAG-3 antibody, an anti-TIGIT antibody, an anti-BTLA antibody, an anti-CTLA4 antibody, an anti-ICOS antibody, an anti-CD27 antibody, an anti-4-1BB antibody, an anti-CD40 antibody, an anti-VEGFR2 antibody, an anti-EGFR antibody, an anti-HER2 antibody, an TIM3 antibody, an CD103 antibody, an TGFBR2 antibody and/or an anti-GITR antibody.

In one aspect, the disclosure provides a combination therapy. In some embodiments, the anti-CD79b antibody or antigen-binding fragment thereof (e.g., any antibody described herein) can be administered together with an anti-CTLA4 antibody.

### Pharmaceutical Compositions and Routes of Administration

Also provided herein are pharmaceutical compositions that contain at least one (e.g., one, two, three, or four) of the antibodies or antigen-binding fragments described herein. Two or more (e.g., two, three, or four) of any of the antibodies or antigen-binding fragments described herein can be present in a pharmaceutical composition in any combination. The pharmaceutical compositions may be formulated in any manner known in the art.

Pharmaceutical compositions are formulated to be compatible with their intended route of administration (e.g., intravenous, intraarterial, intramuscular, intradermal, subcutaneous, or intraperitoneal). The compositions can include a sterile diluent (e.g., sterile water or saline), a fixed oil, polyethylene glycol, glycerine, propylene glycol or other synthetic solvents, antibacterial or antifungal agents, such as benzyl alcohol or methyl parabens, chlorobutanol, phenol, ascorbic acid, thimerosal, and the like, antioxidants, such as ascorbic acid or sodium bisulfite, chelating agents, such as ethylenediaminetetraacetic acid, buffers, such as acetates, citrates, or phosphates, and isotonic agents, such as sugars (e.g., dextrose), polyalcohols (e.g., mannitol or sorbitol), or salts (e.g., sodium chloride), or any combination thereof. Liposomal suspensions can also be used as pharmaceutically acceptable carriers (see, e.g., U.S. Pat. No. 4,522,811). Preparations of the compositions can be formulated and enclosed in ampules, disposable syringes, or multiple dose vials. Where required (as in, for example, injectable formulations), proper fluidity can be maintained by, for example, the use of a coating, such as lecithin, or a surfactant. Absorption of the antibody or antigen-binding fragment thereof can be prolonged by including an agent that delays absorption (e.g., aluminum monostearate and gelatin). Alternatively, controlled release can be achieved by implants and microencapsulated delivery systems, which can include biodegradable, biocompatible polymers (e.g., ethylene vinyl acetate, polyanhydrides, polyglycolic acid, collagen, polyorthoesters, and polylactic acid; Alza Corporation and Nova Pharmaceutical, Inc.).

Compositions containing one or more of any of the antibodies or antigen-binding fragments described herein can be formulated for parenteral (e.g., intravenous, intraarterial, intramuscular, intradermal, subcutaneous, or intraperitoneal) administration in dosage unit form (i.e., physically discrete units containing a predetermined quantity of active compound for ease of administration and uniformity of dosage).

Pharmaceutical compositions for parenteral administration are preferably sterile and substantially isotonic and manufactured under Good Manufacturing Practice (GMP) conditions. Pharmaceutical compositions can be provided in unit dosage form (i.e., the dosage for a single administration). Pharmaceutical compositions can be formulated using one or more physiologically acceptable carriers, diluents, excipients or auxiliaries. The formulation depends on the route of administration chosen. For injection, antibodies can be formulated in aqueous solutions, preferably in physiologically-compatible buffers to reduce discomfort at the site of injection. The solution can contain formulatory agents such as suspending, stabilizing and/or dispersing agents. Alternatively antibodies can be in lyophilized form for constitution with a suitable vehicle, e.g., sterile pyrogen-free water, before use.

Toxicity and therapeutic efficacy of compositions can be determined by standard pharmaceutical procedures in cell cultures or experimental animals (e.g., monkeys). One can, for example, determine the LD50 (the dose lethal to 50% of the population) and the ED50 (the dose therapeutically effective in 50% of the population): the therapeutic index being the ratio of LD50:ED50. Agents that exhibit high therapeutic indices are preferred. Where an agent exhibits an undesirable side effect, care should be taken to minimize potential damage (i.e., reduce unwanted side effects). Toxicity and therapeutic efficacy can be determined by other standard pharmaceutical procedures.

Data obtained from cell culture assays and animal studies can be used in formulating an appropriate dosage of any given agent for use in a subject (e.g., a human). A therapeutically effective amount of the one or more (e.g., one, two, three, or four) antibodies or antigen-binding fragments thereof (e.g., any of the antibodies or antibody fragments described herein) will be an amount that treats the disease in a subject (e.g., kills cancer cells) in a subject (e.g., a human subject identified as having cancer), or a subject identified as being at risk of developing the disease (e.g., a subject who has previously developed cancer but now has been cured), decreases the severity, frequency, and/or duration of one or more symptoms of a disease in a subject (e.g., a human). The effectiveness and dosing of any of the antibodies or antigen-binding fragments described herein can be determined by a health care professional or veterinary professional using methods known in the art, as well as by the observation of one or more symptoms of disease in a subject (e.g., a human). Certain factors may influence the dosage and timing required to effectively treat a subject (e.g., the severity of the disease or disorder, previous treatments, the general health and/or age of the subject, and the presence of other diseases).

Exemplary doses include milligram or microgram amounts of any of the antibodies or antigen-binding fragments described herein per kilogram of the subject's weight (e.g., about 1 g/kg to about 500 mg/kg; about 100 μg/kg to about 500 mg/kg; about 100 g/kg to about 50 mg/kg; about 10 g/kg to about 5 mg/kg; about 10 g/kg to about 0.5 mg/kg; or about 1 g/kg to about 50 μg/kg). While these doses cover a broad range, one of ordinary skill in the art will understand that therapeutic agents, including antibodies and antigen-binding fragments thereof, vary in their potency, and effective amounts can be determined by methods known in the art. Typically, relatively low doses are administered at first, and the attending health care professional or veterinary professional (in the case of therapeutic application) or a researcher (when still working at the development stage) can subsequently and gradually increase the dose until an appropriate response is obtained. In addition, it is understood that the specific dose level for any particular subject will depend upon a variety of factors including the activity of the specific compound employed, the age, body weight, general health, gender, and diet of the subject, the time of administration, the route of administration, the rate of excretion, and the half-life of the antibody or antibody fragment in vivo.

The pharmaceutical compositions can be included in a container, pack, or dispenser together with instructions for administration. The disclosure also provides methods of manufacturing the antibodies or antigen binding fragments thereof for various uses as described herein.

### EXAMPLES

The invention is further described in the following examples, which do not limit the scope of the invention described in the claims.

### Example 1. Generation of Anti-CD79b Antibodies

Rabbit Immunizations with CD79b Antigen

To generate monoclonal antibodies against human CD79b, two New Zealand white rabbits were immunized with human CD79b ECD antigen (SEQ ID NO: 1, aa. 29-159, his-tagged from AcroBiosystems), using either complete Freund's adjuvant or incomplete Freund's adjuvant. Serum titer was monitored by ELISA after the second injection.

### Screening of Single B Cells Producing Monoclonal Antibody to CD79b

After 3-4 immunizations at 200 μg/rabbit/injection, rabbits with good titer (>1:100,000) received a final booster shot and the spleens were harvested 7 days after the final injection.

Isolation of Splenocytes from Rabbits

All the following procedures (except for centrifugation) were performed in a biological safety cabinet. Rabbit spleens were harvested 7 days after the final booster shot. Splenocytes were prepared in a sterile cell strainer placed in the bottom of a 100 mm sterile petri dish containing 20 mL RPMI+1% of Penicillin-Streptomycin (P/S). Using sterile forceps, spleen tissues were transferred to a cell strainer. Specifically, while holding a spleen with forceps, the spleen was cut into small pieces, which were then pressed through the mesh of cell strainer. Tissue debris was washed with 10 mL RPMI+1% P/S. Spleen cells were transferred from the petri dish to a new 50 mL conical tube. RPMI+1% P/S was added to a final volume of 50 ml. Cells were centrifuged at 400×g for 5 min and supernatant was aspirated. 13 mL ACK buffer (Gibco cat #A1049201) was added to re-suspend the cells. The suspended cells were incubated at room temperature for 1 minute. RPMI+1% P/S was added to reach a final volume of 50 ml. Cells were centrifuged at 400×g for 5 min and supernatant was aspirated. The cell pellet was re-suspended in RPMI+10% FBS+1% P/S. The cells were centrifuged at 400×g for 5 min and supernatant was aspirated. The pellet was re-suspended in 15 ml RPMI+10% FBS+1% P/S. The cells were pipetted through 100 μm cell strainer into a 50 mL conical tube to remove cell clumps. Splenocytes were then seeded at desired density (e.g., 4E6 cells/ml) with proper medium for sorting. The remaining splenocytes were frozen in 90% serum+10% DMSO at 6×10⁷ cells/vial (~1.8 ml) at −80° C. overnight. The frozen cells were transferred into liquid nitrogen tank for long term storage.

### Antigen Specific B Cell Sorting

For B cell sorting, freshly isolated or thawed splenocytes (~2×10⁸ splenocytes) were cultured in B-cell culture media (RPMI-1640, 15% FBS, 1×HEPES, 1×2-ME (2-Mercaptoethanol), 1% Penicillin/Streptomycin) overnight before sorting. 96-well B cell feeding plates were prepared accordingly one day before the sorting. On the day of sorting, suspended and loosely attached splenocytes were collected by gently pipetting medium against the culturing surface of flask. The cells were then transferred to a conical tube and centrifuged at 400×g for 3 minutes. The cell pellet was washed with fluorescence-activated cell sorting (FACS) buffer (1×PBS+0.5% BSA) twice. Biotinylated antigen was added at 5 μg/ml (final concentration). The mixture was incubated at room temperature (RT) for 20 min. The staining mixture was then centrifuged at 400×g for 3 min and the cells were re-suspended in FACS buffer. The cells were transferred into a 1.5 ml amber Eppendorf tube. The staining antibody mixtures were then added to the cells. The staining mixture was incubated at 4° C. for 15-30 min, and then centrifuged at 400×g for 3 min. The cell pellet was washed twice with FACS buffer. The washed cell pellet was re-suspended at ~10⁷ cells/ml in 1×PBS+1% FBS. Using Fluorescence-activated cell sorting, antigen specific single B cells were sorted into 96-well plates (20 plates for each rabbit). 96-well B cell culture plates with sorted B cells were cultured in 37° C. with 5% CO₂ for 12 days.

### Screening of Single B Cell Culture

On day 8 post sorting, 15 μL of B cell culture supernatant was collected from each well for antigen specific ELISA. Briefly, the B cell culture supernatants were transferred to a CD79b extracellular domain (ECD) coated, then blocked 384-well plate. The cells were incubated at room temperature for 1 hour, and washed 3× with PBS plus 0.05% Tween-20. Antibody secreted were detected using goat anti-rabbit IgG HR+TMB substrate. B cell supernatants that met the cut off OD450 (>0.5 or 3× higher than the pre-immune serum) were then selected. Using FACS, the selected cells were (1) counter-screened against a CD79b variant with N-terminal deletion (deleting the first 13 amino acid residues from the N-terminus, SEQ ID NO: 2); (2) screened against Cynomolgus CD79b (SEQ ID NO: 3) for cross-species binding, and (3) screened against Daudi cells for cell surface binding.

Initial ELISA screening of supernatants from 40 plates (96-well plate) sorted B cell were screened for CD79b specific antibodies. Approximately 230 CD79b antigen specific positive B cell clones were identified.

On day 12 post sorting, the B cell culture plates were centrifuged at 400×g for 3 min. Supernatants from positive clones (with OD greater than selected cutoff from antigen specific ELISA) were collected and the cell pellets were preserved in 100 μL DNA/RNA shield (Zymo Cat #R1100-250) in 250 μL PCR tubes. Collected supernatants were subjected to additional tests as described below.

Screening of CD79b Specific Antibodies that Bind to Cell Surface Expressed CD79b

CD79b is a member of the B cell receptor complex. It is anticipated that not all antibody clones that bind to CD79b in ELISA would also bind to cell surface expressed CD79b due to epitope accessibility. The CD79b specific B cell clones identified by ELISA were subjected to cell surface binding assay with Daudi cells by FACS. Briefly, 50 μl of the B cell supernatant (1:10 dilution in FACS buffer, 1×PBS+0.5% BSA) was incubated with 50,000 Daudi cells (plated in a 96 well plate) on ice for 1 hour, and washed 2× with 150 μl of ice cold FACS buffer. 100 μl of anti-rabbit-PE secondary antibody (Biolegend) was added, mixed and incubated on ice for 30 minutes. The cells were washed 3× with 150 μl of FACS buffer and finally re-suspend in 100 μl of FACS buffer. 5,000 cells from each staining were collected in a Flow Cytometer for analysis. Of the 230 or so ELISA positive B cell clones, 12 clones were shown to bind to CD79b on cell surface.

Recovery of VH and VL Gene Sequences from Selected Anti-CD79b Clones

DNA fragments encoding the heavy chain variable domain (VH) and light chain variable domain (VL) from B cell clones showing high cell surface binding affinity to CD79b were amplified via 5′ RACE (rapid amplification of cDNA ends), TOPO cloned, and sequenced.

### Example 2. Generation of Chimeric Expression Constructs

To express rabbit/human chimeric IgG1 antibodies, constant regions of the heavy chain (CH1-CH3, SEQ ID NO: 4) of human IgG1 and the constant region of human Kappa light chain (CL-kappa, SEQ ID NO: 5) were synthesized and cloned into pcDNA3.4 vectors (Invitrogen) respectively. The heavy chain cloning vector, pcDNA3.4-huIgG1-Hc, was digested with EcoRI/NheI for the cloning of VH fragment. The light chain cloning vector, pcDNA3.4-huKappa-Lc, was digested with EcoRI/BsiWI for the cloning of VL fragment. Recombinant rabbit/human chimeric antibody constructs were generated. Specifically, selected VH and VL sequences from rabbit anti-CD79b antibodies (FIG. 22) were obtained by gene synthesis (Integrated DNA technology), and linked to a leader sequence (SEQ ID NO: 6) for secretion with overlapping sequences at both 5′ and 3′ ends for Gibson assembly (NEB NEBuilder® HiFi DNA Assembly) to generate the HC and LC expression plasmids. The assembled plasmids were used to transform competent E. coli (NEB® 5-alpha). The clones with correct sequence were selected based on sequencing results (Elim Biopharm), and were further cultured with LB containing Carbenicillin (100 ug/ml). Plasmids were purified (QIAGEN Plasmid Plus Kits), eluted in Nuclease-free H₂O (Sigma), and stored at −80° C.

### Example 3. Expression and Purification of Chimeric Anti-CD79b Antibodies

Recombinant chimeric antibodies were expressed in CHO cells (ExpiCHO™ Expression System, Gibco) by transfecting the cells with the pcDNA3.4-huIgG1-Hc and pcDNA3.4-huKappa-Lc containing paired VH and VL sequences. ExpiCHO cell was cultured with ExpiCHO expression medium and maintained at 0.3 to 6×10⁶ cells/ml, 37° C., 125 rpm, 5% CO₂ and 80% humidity. In a 125 ml baffled flask, 25 ml of fresh ExpiCHO cells (6×10⁶/ml and viability>95%, prepared the day before transfection) were seeded at 3×10⁶/ml. 1 ml of serum-free medium (OptiPRO™ SFM, Gibco) containing both pcDNA3.4-huIgG1-Hc and pcDNA3.4-huKappa-Lc (12 ug each plasmid) was mixed well by pipetting with 1 ml OptiPRO™ SFM containing 80 ul transfection reagent (ExpiFectamine™ CHO Reagent, Gibco). The transfection mixture was then added to the 25 ml ExpiCHO cells and cultured at 37° C. The next day, the transfection culture was transferred to 32° C. incubator after the addition of 150 ul ExpiFectamine™ CHO Enhancer, 6 ml ExpiCHO™ Feed and 1× Penicillin-Streptomycin (Gibco). The cell density and viability of the transfection culture were monitored and the IgG1 antibody titer in the medium was monitored using a Bio-layer interferometry (BLI) instrument with Protein Abiosensor (Gator prime by Gator Bio).

5 days later, the culture medium containing secreted IgG1 antibodies were collected (centrifugation at 2000 g for 10 minutes), filtered (Thermo Scientific™ Nalgene™ Rapid-Flow™ Sterile Disposable Filter) and further purified using a Protein A resin (TOYOPEARL AF-rProtein A Hc-650F) packed gravity-flow column (Bio-Rad). IgG1 antibodies were eluted with 3.5 ml Glycine-HCl (100 mM, pH 2.7), immediately neutralized with 1M Tris-HCl (pH8.5), dialyzed with Thermo Scientific™ Slide-A-Lyzer™ G2 Dialysis Cassettes (20K MWCO) in 1×PBS buffer (Ph 7.2), and stored at 4° C. The concentration of purified IgG1 antibody was determined with NanoDrop™ One/Onec Microvolume UV-Vis Spectrophotometer (Thermo ScientificT^{M}) and the quality of IgG1 antibody was checked by SDS-PAGE gel under both denaturing and non-denaturing conditions.

### Example 4. Binding Affinities of Anti-CD79b Antibodies to Human and Cynomolgus CD79b Recombinant Proteins as Determined by ELISA and BLI

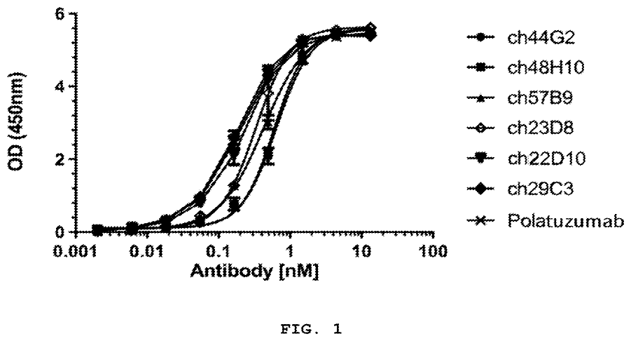

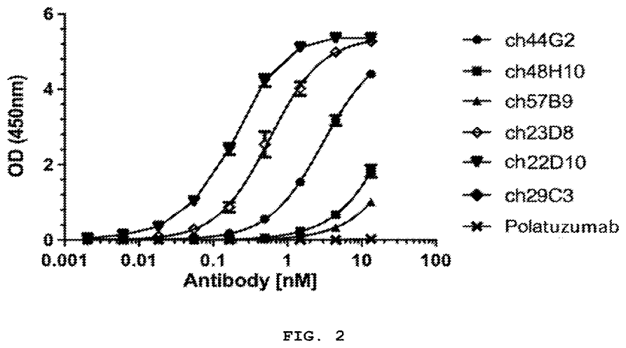

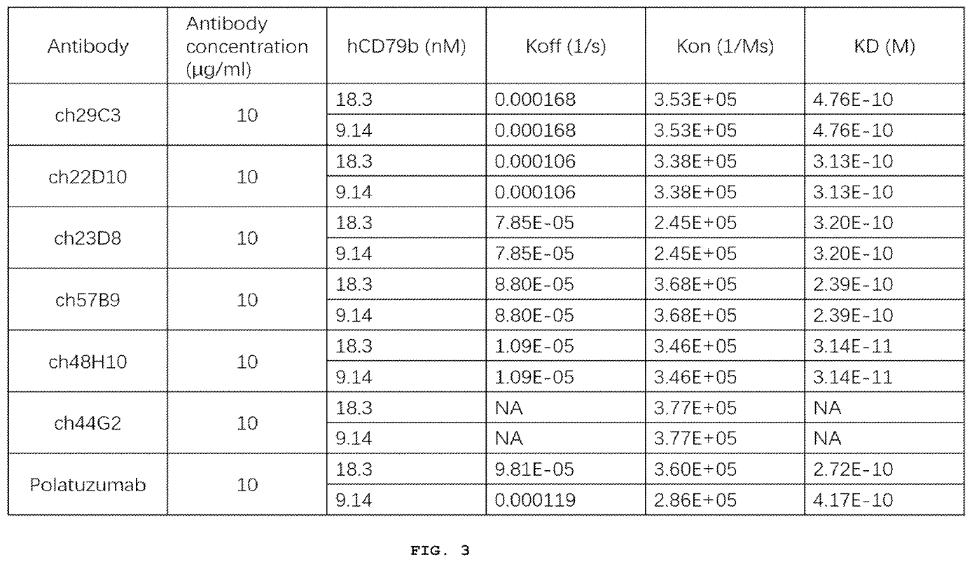

Binding affinities of anti-CD79b antibodies to human and cynomolgus CD79b recombinant proteins were tested by ELISA. 1 ug/ml (in PBS buffer) of human or cynomolgus CD79b recombinant proteins were coated on an ELISA plate overnight. ELISA plates were washed and blocked with blocking buffer (PBS+1% BSA) followed by incubation with serially diluted anti-CD79b primary antibodies. Anti-CD79b antibody binding was quantified using an anti-human IgG HRP secondary antibody (Biolegend Catalog #410902) and HRP substrate. The ELISA binding affinities of anti-CD79b antibodies to human and cynomolgus CD79b recombinant proteins are shown in Table 1, FIG. 1 and FIG. 2.

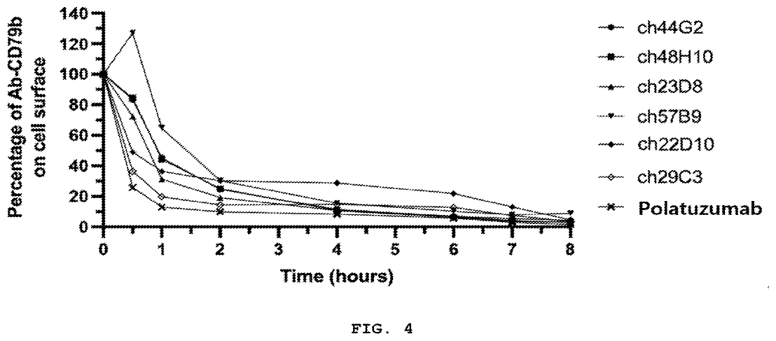

Binding affinities of anti-CD79b antibodies to human CD79b recombinant proteins were tested by BLI. 10 ug/ml of anti-CD79b antibodies were captured by anti-human IgG Fc probes (Gator Bio Catalog #160024). Association and dissociation of serially diluted (146 nM to 0 nM, 2-fold dilution in PBS+0.05% Tween-20) human CD79b recombinant protein were measured. Absolute kD was determined by applying a 1:1 binding model (Global Rmax unlinked). The BLI binding affinities of anti-CD79b antibodies to human CD79b recombinant proteins are shown in Table 1, FIG. 3. Polatuzumab was included for comparison purpose. With respect to ch44G2, according to the BLI data, Kon is 3.77E+05, but koff is not detectable. It suggests ch44G2 has a strong binding affinity with CD79b.

TABLE 1
ELISA IC50 (nM) binding of anti-CD79b antibodies to human and
cynomolgus CD79b recombinant proteins and BLI kD (M) binding
of anti-CD79b antibodies to human recombinant protein

| | ELISA IC50 (nM) | ELISA IC50 (nM) | BLI KD (M) |
|---|---|---|---|
| antibody | Human CD79b | Cynomolgus CD79b | Human CD79b |
| ch29C3 | 0.17 | No binding | 4.76E−10 |
| ch22D10 | 0.22 | 0.19 | 3.13E−10 |
| ch23D8 | 0.33 | 0.57 | 3.20E−10 |
| ch57B9 | 0.42 | 186.5 | 2.39E−10 |
| ch48H10 | 0.60 | 65.32 | 3.14E−11 |
| ch44G2 | 0.63 | 3.0 | Over the range |
| polatuzumab | 0.19 | No binding | 2.72E−10 |

### Example 5. The Internalization of Anti-CD79b Antibodies

The internalization of anti-CD79b antibodies was evaluated. About 50,000 Ramos cells (for each time point) were incubated with 20 ug/ml of labeled anti-CD79b antibodies and incubated at 37° C. At different time points, cells were washed and the presence of anti-CD79b antibody-bound receptor was detected using an anti-human IgG PE secondary antibody (Jackson Research Catalog #109-116-170). For analysis, the mean fluorescence intensity (MFI (PE)) of each antibody was normalized to its respective zero time point (100%) and a decrease in percentage MFI (PE) over time was plotted. As shown in FIG. 4, all the screened anti-CD79b antibodies have slower internalization than polatuzumab. Among them, ch23D8, ch44G2, ch48H10, and ch57B9 exhibit much slower internalization process than other antibodies.

### Example 6. Quantification of Tumor Cell Surface CD79b Antigen Densities

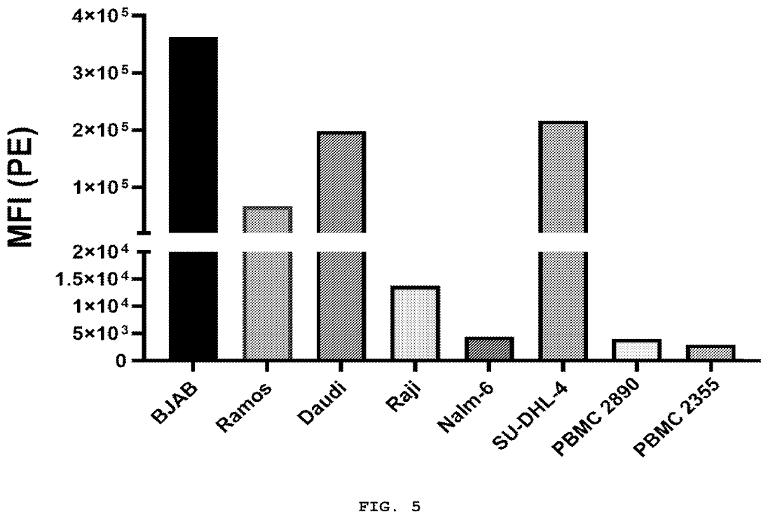

To estimate CD79b antigen expression levels on malignant B tumor cell and normal B cell surface, cell lines and PBMCs from two healthy donors were stained at a saturating concentration of anti-CD79b antibody ch44G2 on ice for 30 min. After being washed with PBS, the cells were stained with a secondary PE-labeled anti-human IgG (Invitrogen, Cat: 12-4998-82) for 30 min on ice. Cells were then washed with PBS and resuspended in FACS buffer for flow cytometry analysis using Cytek northern lights. Results were analyzed and graphed using GraphPad prism software (Version 9.4.1; GraphPad Software Inc.). The quantification of CD79b antigen density on malignant B tumor cell and normal B cell surface are shown in Table 2 and FIG. 5.

TABLE 2
Quantification of CD79b antigen density on malignant B tumor
cell and normal B cell surface

| | | | | | Nalm- | SU-DHL- | PBMC | PBMC |
|---|---|---|---|---|---|---|---|---|
| | BJAB | Ramos | Daudi | Raji | 6 | 4 | 2890 | 2355 |
| MFI(PE) | 362371 | 67352 | 198501 | 13782 | 4460 | 255605 | 4024 | 2939 |

### Example 7. Malignant B Cell Line Binding Assay

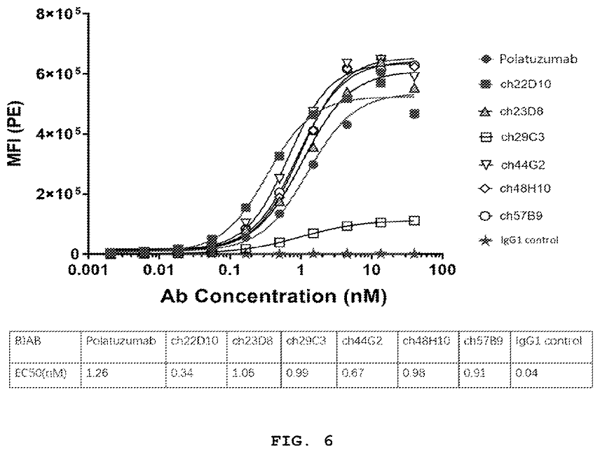

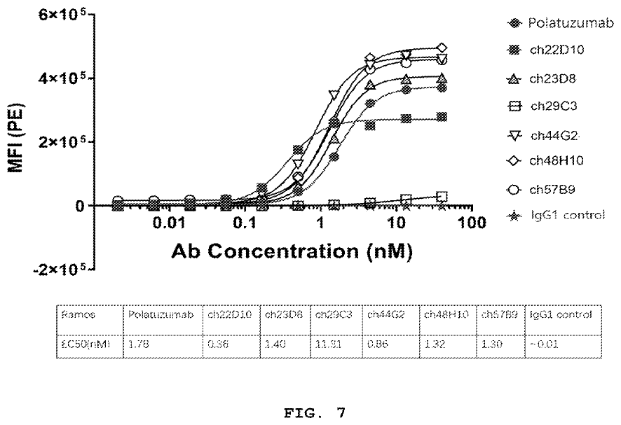

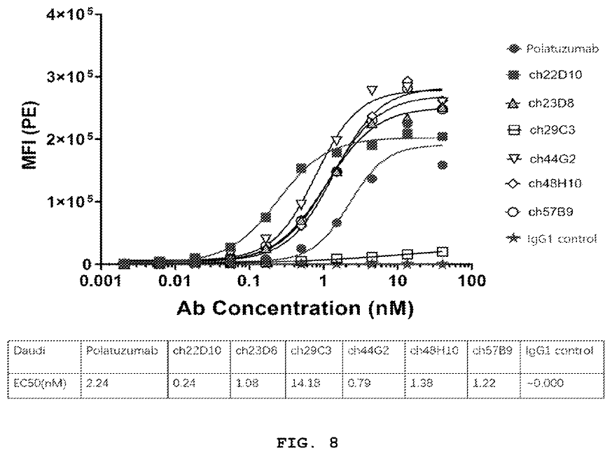

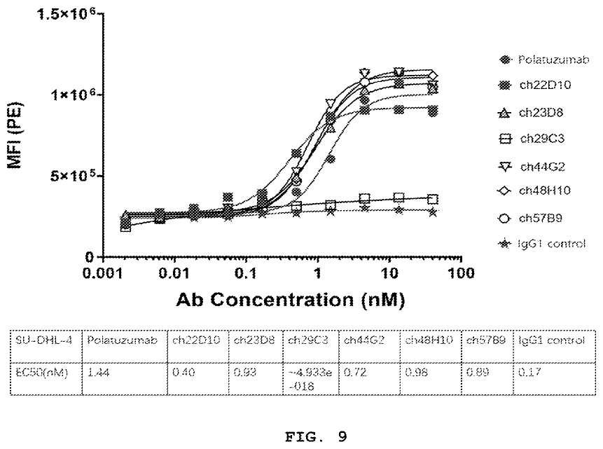

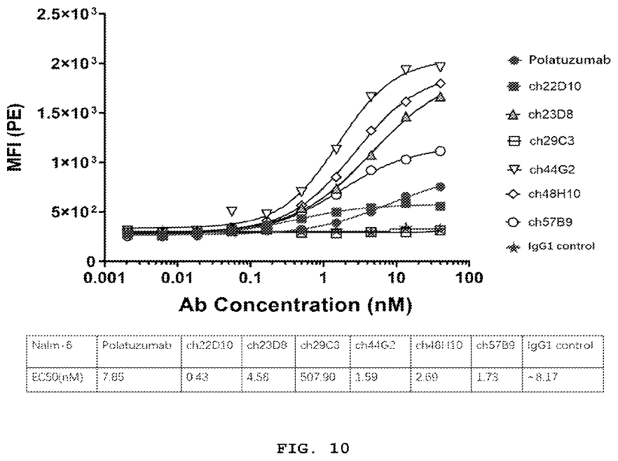

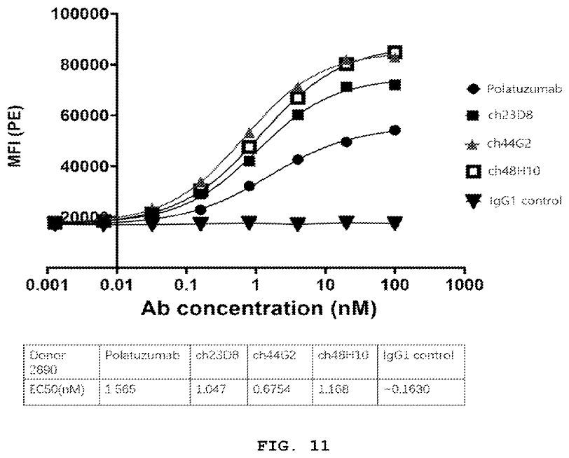

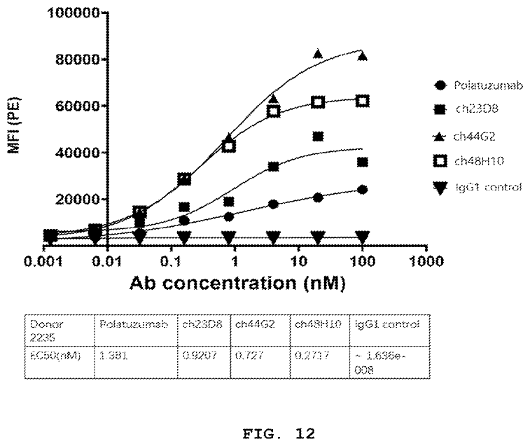

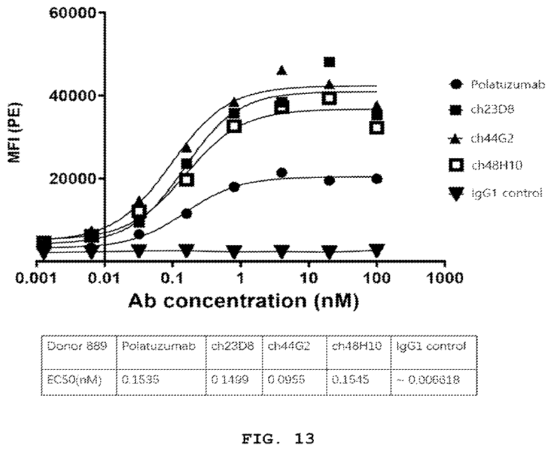

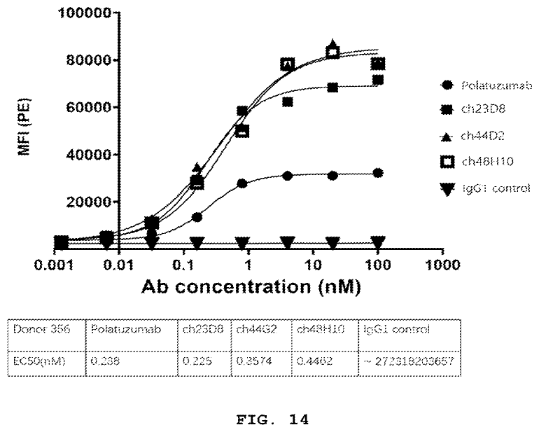

The binding affinities of anti-CD79b antibodies to malignant B cell lines were tested. Polatuzumab and IgG1 were used as the positive control and the negative control, respectively. Malignant B cell lines (BJAB, Ramos, Daudi, SU-DHL-4 and Nalm-6) at 50,000 cells/well in a 96 well plate were incubated with anti-CD79b antibodies (ch22D10, ch23D8, ch29C3, ch44G2, ch48H10, ch57B9 and polatuzumab) and IgG1 (Biolegend, Cat #:403502) at different concentrations (serial 1:3 dilutions from 40 nM) for 30 min on ice, and washed twice with 200 ul PBS. The cells were then incubated with 100 μl anti-human IgG1-PE (Invitrogen, cat: 12-4998-82), 1:1000 dilution, for 30 min on ice, washed with 200 ul PBS for three times before flow cytometry analysis. As shown in FIGS. 6-10, anti-CD79b antibodies ch23D8, ch44G2, ch48H10, and ch57B9 showed higher cell surface binding affinities (lower EC50) than Polatuzumab to BJAB (FIG. 6), Ramos (FIG. 7), Daudi (FIG. 8), SU-DIL-4 (FIG. 9) and Nalm-6 (FIG. 10). The results demonstrated that ch23D8, ch44G2, ch48H10, and ch57B9 can bind to malignant B cell lines with both high and low CD79b antigen densities.

### Example 8. Endogenous B Cell Binding Assay

The binding affinities of anti-CD79b antibodies to endogenous B cells were tested. Polatuzumab and IgG1 control were used as the positive control and the negative control, respectively. PBMCs from 4 donors (Stanford blood center) at 200,000 cells/well in a 96 well plate were incubated with anti-CD79b antibodies (ch23D8, ch44G2, ch48H10 and polatuzumab) and IgG1 (Biolegend, Cat #: 403502) at different concentrations (serial 1:5 dilution from 50 nM) for 30 min on ice, and washed twice with 200 ul PBS. The cells were then incubated with 100 pd mixture of anti-human IgG1-PE (Invitrogen, Cat #: 12-4998-82), 1:1000 dilution; CD19-BV421 (Biolegend, Cat #:302230, 1:400 dilution) for 30 min on ice, and washed with 200 ul PBS for three times before flow cytometry analysis. As shown in FIGS. 11-14, ch44G2, ch48H10, ch23D8 showed higher cell surface binding affinities to endogenous B cells in all 4 different donors than Polatuzumab.

### Example 9. CLL Patients' PBMC Binding Assay

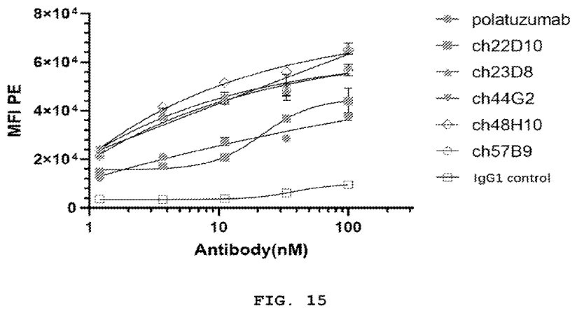

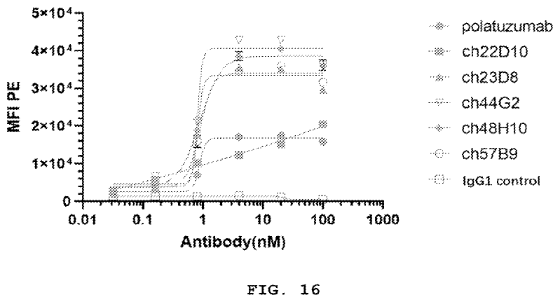

To determine the binding affinities of the anti-CD79b antibodies to cell surface CD79b on Chronic Lymphocytic Leukemia (CLL) patient's B lymphocytes, a cell-based binding assay was conducted. Polatuzumab and IgG1 control were used as positive and negative controls, respectively. CLL patient PBMCs were purchased from the company Bioscience. FACS analysis demonstrated that 95% of B lymphocytes (CD19+) in the PBMCs were positive. Specifically, 2.5×10⁴ PBMC cells in 50 μl FACS buffer (1×PBS+2% BSA+2 mM EDTA) were seeded in each well of a V bottom 96-well plate. Then, 50 μl of serially diluted anti-CD79b antibody (ch22D10, ch23D8, ch29C3, ch44G2, ch48H10, ch57B9 or polatuzumab) or IgG1 was added into each well. The mixture was incubated on ice for 30 minutes, and then washed twice with FACS buffer. The cells were re-suspended in 100 μl FACS buffer containing PE conjugated goat anti-human IgG Fc (1:1000) and anti-human CD19 (BV421), and incubated for 30 minutes on ice in darkness. After washing twice with FACS buffer, the cells were re-suspended in 100 μl of FACS buffer and analyzed with a Cytek cytometer. As shown in FIGS. 15-16, ch23D8, ch44G2, ch48H10 and ch57B9 exhibited more potent binding to CLL patient's B lymphocytes than polatuzumab.

### Example 10. CD79b mAb Binding to Long and Short Isoform of CD79b

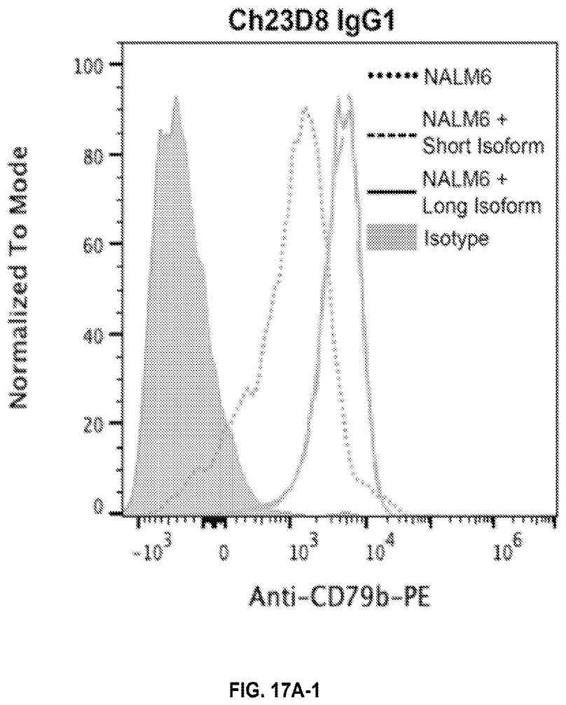

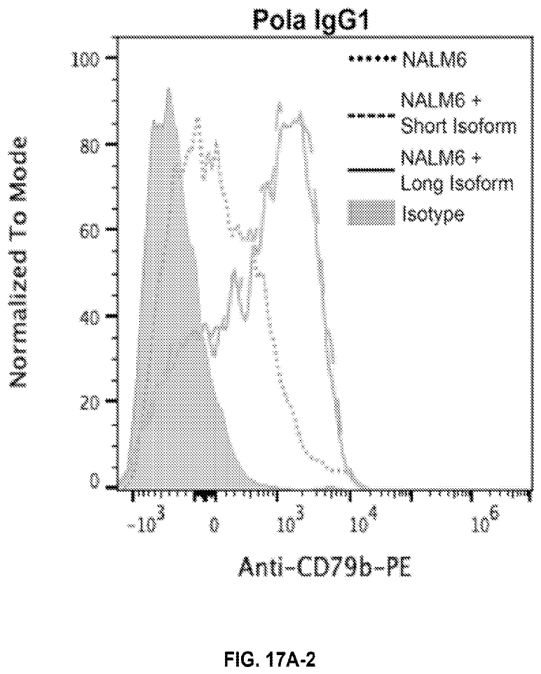

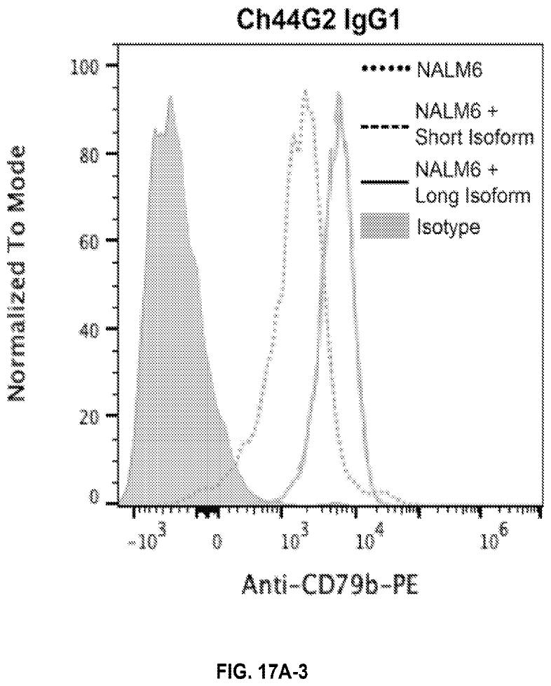

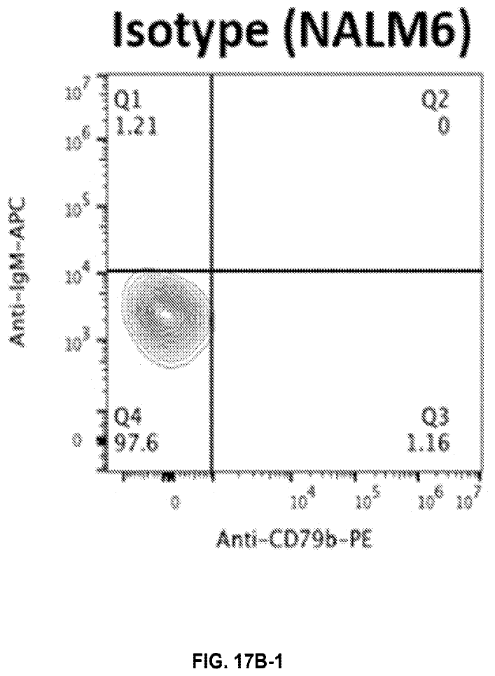

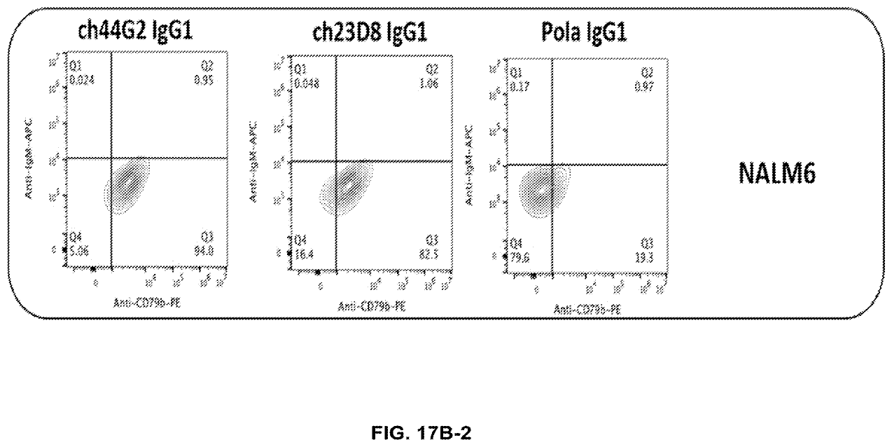

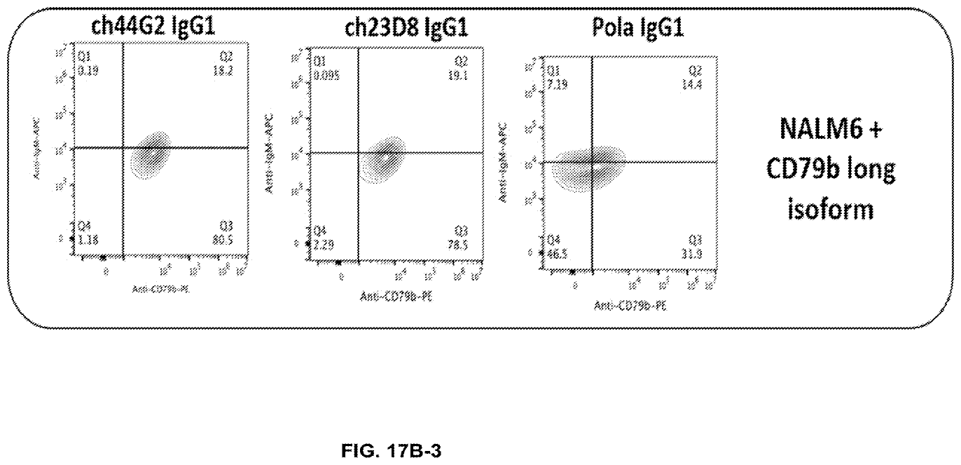

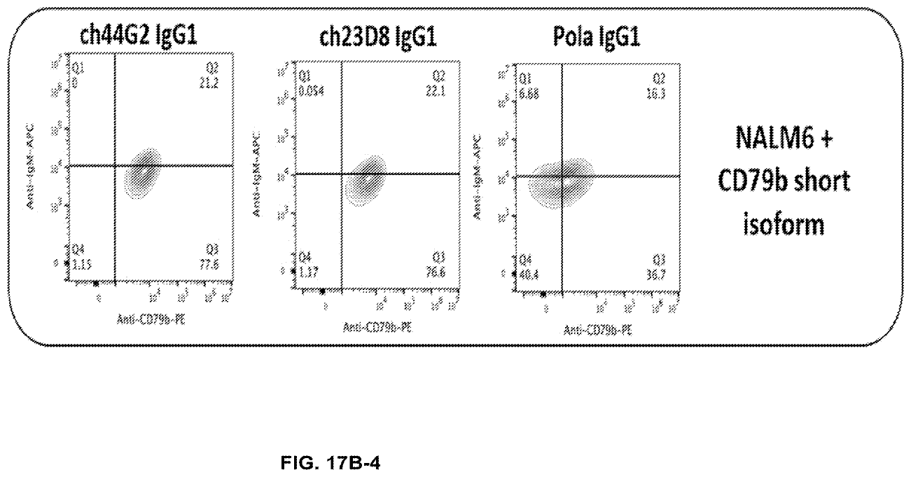

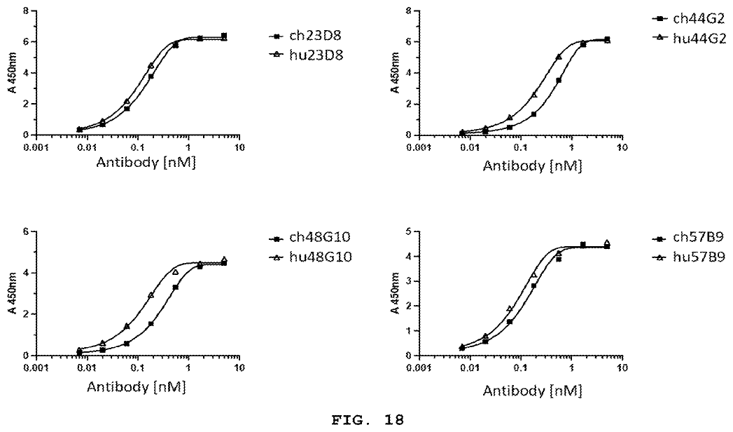

The surface binding of the anti-CD79b antibodies to both short and long isoforms of CD79b was conducted. Polatuzumab was used as positive controls. About 2 million Nalm-6 cells were electroporated with expression plasmids encoding the short (Nalm-6+short isoform) and long isoforms (Nalm-6+long isoform) of CD79b separately. After 48 hours, cells were stained with indicated anti-CD79b antibodies (ch23D8, ch44G2 or polatuzumab) and detected using an anti-IgM-APC (BD bioscience, Catalog #551062). As shown in FIGS. 17A-17B, ch23D8 and ch44G2 have a strong binding affinity with long isoforms CD79b as well as short isoforms CD79b.

### Example 11. Humanization of Anti-CD79b Antibodies

Four rabbit anti-human CD79b monoclonal antibody clones, 23D8, 44G2, 48H10 and 57B9 were humanized by grafting the CDRs of the leading antibodies into selected human germline frameworks closest to the rabbit frameworks identified by IgBLAST (https://www.ncbi.nlm.nih.gov/igblast/) and/or IMGT/DomainGapAlig (https://www.imgt.org/3Dstructure-DB/cgi/DomainGapAlign.cgi) with. Human germline IGHV, IGKV, IGHJ and IGKJ were selected based on sequence similarity within both frameworks and CDRs. Certain human germline framework residues were back mutated to corresponding rabbit residues to maintain canonical loop structure and light chain/heavy chain interface (Padlan Mol. Immunol., 1994, 31:169; Foote and Winter J M B, 1992, 224:487; Padlan Mol. Immunol., 1994, 31:169). By humanization, hu23D8, hu44G2, hu48H10 and hu57B9 humanized antibody were generated.

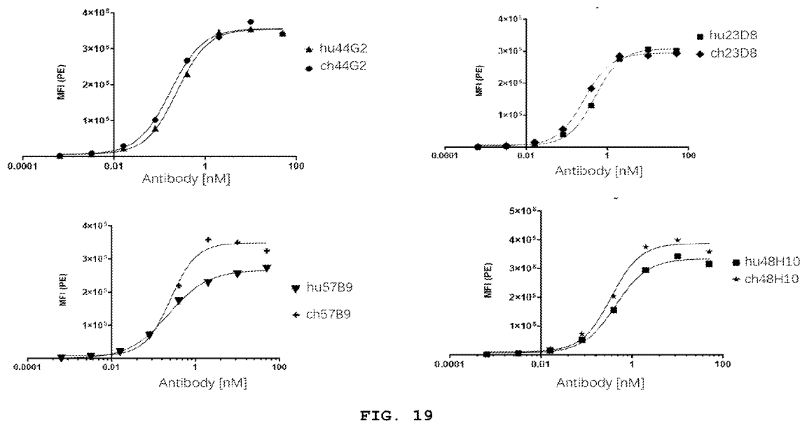

Thermostability of humanized antibody clones were assessed by Nano DSF (Table 3). The humanized antibodies were confirmed to bind to recombinant human CD79b ECD in ELISA (FIG. 18), and cell lines expressing endogenous CD79b (FIG. 19).

The results showed that the humanized anti-CD79b antibodies showed the identical technical effects as the chimeric anti-CD79b antibodies, such as high affinity to CD79b, slow internalization, high binding to malignant B cell lines with both high and low CD79b antigen densities, high cell surface binding affinities to endogenous B cells, high binding to CLL patient's B lymphocytes and strong binding affinity with long isoforms CD79b as well as short isoforms CD79b.

TABLE 3
Thermostability of humanized anti-CD79b antibodies

Nano DSF

| Antibodies | Tm1 (° C.) | Tm2 (° C.) |
|---|---|---|
| ch23D8 | 63.0 | 81.2 |
| ch44G2 | 71.0 | 81.0 |
| ch48H10 | 67.8 | 80.8 |
| ch57B9 | 70.7 | 77.5 |
| hu23D8 | 58.7 | 81.1 |
| hu44G2 | 68.2 | 84.0 |
| hu48H10 | 66.6 | 81.2 |
| hu57B9 | 69.1 | 81.5 |

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An antibody or antigen-binding fragment thereof that binds to CD79b (Cluster of Differentiation 79B) comprising:
a heavy chain variable region (VH) comprising complementarity determining regions (CDRs) 1, 2, and 3, wherein the VH CDR1 region comprises an amino acid sequence that is at least 80% identical to a selected VH CDR1 amino acid sequence, the VH CDR2 region comprises an amino acid sequence that is at least 80% identical to a selected VH CDR2 amino acid sequence, and the VH CDR3 region comprises an amino acid sequence that is at least 80% identical to a selected VH CDR3 amino acid sequence; and
a light chain variable region (VL) comprising CDRs 1, 2, and 3, wherein the VL CDR1 region comprises an amino acid sequence that is at least 80% identical to a selected VL CDR1 amino acid sequence, the VL CDR2 region comprises an amino acid sequence that is at least 80% identical to a selected VL CDR2 amino acid sequence, and the VL CDR3 region comprises an amino acid sequence that is at least 80% identical to a selected VL CDR3 amino acid sequence,
wherein the selected VH CDRs 1, 2, and 3 amino acid sequences and the selected VL CDRs, 1, 2, and 3 amino acid sequences are one of the following:
(1) the selected VH CDRs 1, 2, 3 amino acid sequences are set forth in SEQ ID NOs: 39, 41, 43, respectively, and the selected VL CDRs 1, 2, 3 amino acid sequences are set forth in SEQ ID NOs: 44-46, respectively;
(2) the selected VH CDRs 1, 2, 3 amino acid sequences are set forth in SEQ ID NOs: 9, 11, 13, respectively, and the selected VL CDRs 1, 2, 3 amino acid sequences are set forth in SEQ ID NOs: 14-16, respectively;
(3) the selected VH CDRs 1, 2, 3 amino acid sequences are set forth in SEQ ID NOs: 19, 21, 23, respectively, and the selected VL CDRs 1, 2, 3 amino acid sequences are set forth in SEQ ID NOs: 24-26, respectively;
(4) the selected VH CDRs 1, 2, 3 amino acid sequences are set forth in SEQ ID NOs: 29, 31, 33, respectively, and the selected VL CDRs 1, 2, 3 amino acid sequences are set forth in SEQ ID NOs: 34-36, respectively;
(5) the selected VH CDRs 1, 2, 3 amino acid sequences are set forth in SEQ ID NOs: 49, 51, 53, respectively, and the selected VL CDRs 1, 2, 3 amino acid sequences are set forth in SEQ ID NOs: 54-56, respectively;
(6) the selected VH CDRs 1, 2, 3 amino acid sequences are set forth in SEQ ID NOs: 59, 61, 63, respectively, and the selected VL CDRs 1, 2, 3 amino acid sequences are set forth in SEQ ID NOs: 64-66, respectively;
(7) the selected VH CDRs 1, 2, 3 amino acid sequences are set forth in SEQ ID NOs: 40, 42, 43, respectively, and the selected VL CDRs 1, 2, 3 amino acid sequences are set forth in SEQ ID NOs: 44-46, respectively;
(8) the selected VH CDRs 1, 2, 3 amino acid sequences are set forth in SEQ ID NOs: 10, 12, 13, respectively, and the selected VL CDRs 1, 2, 3 amino acid sequences are set forth in SEQ ID NOs: 14-16, respectively;
(9) the selected VH CDRs 1, 2, 3 amino acid sequences are set forth in SEQ ID NOs: 20, 22, 23, respectively, and the selected VL CDRs 1, 2, 3 amino acid sequences are set forth in SEQ ID NOs: 24-26, respectively;
(10) the selected VH CDRs 1, 2, 3 amino acid sequences are set forth in SEQ ID NOs: 30, 32, 33, respectively, and the selected VL CDRs 1, 2, 3 amino acid sequences are set forth in SEQ ID NOs: 34-36, respectively;
(11) the selected VH CDRs 1, 2, 3 amino acid sequences are set forth in SEQ ID NOs: 50, 52, 53, respectively, and the selected VL CDRs 1, 2, 3 amino acid sequences are set forth in SEQ ID NOs: 54-56, respectively; and
(12) the selected VH CDRs 1, 2, 3 amino acid sequences are set forth in SEQ ID NOs: 60, 62, 63, respectively, and the selected VL CDRs 1, 2, 3 amino acid sequences are set forth in SEQ ID NOs: 64-66, respectively.

2.-13. (canceled)

14. The antibody or antigen-binding fragment thereof of claim 1, wherein the antibody or antigen-binding fragment thereof specifically binds to human, mouse, monkey, or dog CD79b.

15. The antibody or antigen-binding fragment thereof of claim 1, wherein the antibody or antigen-binding fragment thereof is a humanized antibody or antigen-binding fragment thereof, a single-chain variable fragment (scFv), a one-armed antibody, and/or a multi-specific antibody (e.g., a bispecific antibody).

16. The antibody or antigen-binding fragment thereof of claim 1, wherein the antibody or antigen-binding fragment thereof comprises a human IgG1 constant region, a human IgG2 constant region, or a human IgG4 constant region.

17. A nucleic acid comprising a polynucleotide encoding a polypeptide comprising:
(1) an immunoglobulin heavy chain or a fragment thereof comprising a VH comprising complementarity determining regions (CDRs) 1, 2, and 3 comprising the amino acid sequences set forth in SEQ ID NOs: 39, 41, 43, respectively, and wherein the VH, when paired with a light chain variable region (VL) comprising the amino acid sequence set forth in SEQ ID NO: 38 binds to CD79b;
(2) an immunoglobulin light chain or a fragment thereof comprising a VL comprising CDRs 1, 2, and 3 comprising the amino acid sequences set forth in SEQ ID NOs: 44-46, respectively, and wherein the VL, when paired with a VH comprising the amino acid sequence set forth in SEO ID NO: 37 binds to CD79b;
(3) an immunoglobulin heavy chain or a fragment thereof comprising a VH comprising complementarity determining regions (CDRs) 1, 2, and 3 comprising the amino acid sequences set forth in SEQ ID NOs: 9, 11, 13, respectively, and wherein the VH, when paired with a light chain variable region (VL) comprising the amino acid sequence set forth in SEQ ID NO: 8 binds to CD79b;
(4) an immunoglobulin light chain or a fragment thereof comprising a VL comprising complementarity determining regions (CDRs) 1, 2, and 3 comprising the amino acid sequences set forth in SEQ ID NOs: 14-16, respectively, and wherein the VL, when paired with a VH comprising the amino acid sequence set forth in SEQ ID NO: 7 binds to CD79b;
(5) an immunoglobulin heavy chain or a fragment thereof comprising a VH comprising complementarity determining regions (CDRs) 1, 2, and 3 comprising the amino acid sequences set forth in SEQ ID NOs: 19, 21, 23, respectively, and wherein the VH, when paired with a light chain variable region (VL) comprising the amino acid sequence set forth in SEQ ID NO: 18 binds to CD79b;
(6) an immunoglobulin light chain or a fragment thereof comprising a VL comprising complementarity determining regions (CDRs) 1, 2, and 3 comprising the amino acid sequences set forth in SEQ ID NOs: 24-26, respectively, and wherein the VL, when paired with a VH comprising the amino acid sequence set forth in SEQ ID NO: 17 binds to CD79b;
(7) an immunoglobulin heavy chain or a fragment thereof comprising a VH comprising complementarity determining regions (CDRs) 1, 2, and 3 comprising the amino acid sequences set forth in SEQ ID NOs: 29, 31, 33, respectively, and wherein the VH, when paired with a light chain variable region (VL) comprising the amino acid sequence set forth in SEQ ID NO: 28 binds to CD79b;
(8) an immunoglobulin light chain or a fragment thereof comprising a VL comprising CDRs 1, 2, and 3 comprising the amino acid sequences set forth in SEQ ID NOs: 34-36, respectively, and wherein the VL, when paired with a VH comprising the amino acid sequence set forth in SEQ ID NO: 27 binds to CD79b;
(9) an immunoglobulin heavy chain or a fragment thereof comprising a VH comprising complementarity determining regions (CDRs) 1, 2, and 3 comprising the amino acid sequences set forth in SEQ ID NOs: 49, 51, 53, respectively, and wherein the VH, when paired with a light chain variable region (VL) comprising the amino acid sequence set forth in SEQ ID NO: 48 binds to CD79b;
(10) an immunoglobulin light chain or a fragment thereof comprising a VL comprising CDRs 1, 2, and 3 comprising the amino acid sequences set forth in SEQ ID NOs: 54-56, respectively, and wherein the VL, when paired with a VH comprising the amino acid sequence set forth in SEQ ID NO: 47 binds to CD79b;
(11) an immunoglobulin heavy chain or a fragment thereof comprising a VH comprising complementarity determining regions (CDRs) 1, 2, and 3 comprising the amino acid sequences set forth in SEQ ID NOs: 59, 61, 63, respectively, and wherein the VH, when paired with a light chain variable region (VL) comprising the amino acid sequence set forth in SEQ ID NO: 58 binds to CD79b;
(12) an immunoglobulin light chain or a fragment thereof comprising a VL comprising CDRs 1, 2, and 3 comprising the amino acid sequences set forth in SEQ ID NOs: 64-66, respectively, and wherein the VL, when paired with a VH comprising the amino acid sequence set forth in SEQ ID NO: 57 binds to CD79b;
(13) an immunoglobulin heavy chain or a fragment thereof comprising a VH comprising complementarity determining regions (CDRs) 1, 2, and 3 comprising the amino acid sequences set forth in SEQ ID NOs: 40, 42, 43, respectively, and wherein the VH, when paired with a light chain variable region (VL) comprising the amino acid sequence set forth in SEQ ID NO: 38 binds to CD79b;
(14) an immunoglobulin heavy chain or a fragment thereof comprising a VH comprising complementarity determining regions (CDRs) 1, 2, and 3 comprising the amino acid sequences set forth in SEQ ID NOs: 10, 12, 13, respectively, and wherein the VH, when paired with a light chain variable region (VL) comprising the amino acid sequence set forth in SEQ ID NO: 8 binds to CD79b;
(15) an immunoglobulin heavy chain or a fragment thereof comprising a VH comprising complementarity determining regions (CDRs) 1, 2, and 3 comprising the amino acid sequences set forth in SEO ID NOs: 20, 22, 23, respectively, and wherein the VH, when paired with a light chain variable region (VL) comprising the amino acid sequence set forth in SEQ ID NO: 18 binds to CD79b;
(16) an immunoglobulin heavy chain or a fragment thereof comprising a VH comprising complementarity determining regions (CDRs) 1, 2, and 3 comprising the amino acid sequences set forth in SEQ ID NOs: 30, 32, 33, respectively, and wherein the VH, when paired with a light chain variable region (VL) comprising the amino acid sequence set forth in SEQ ID NO: 28 binds to CD79b;
(17) an immunoglobulin heavy chain or a fragment thereof comprising a VH comprising complementarity determining regions (CDRs) 1, 2, and 3 comprising the amino acid sequences set forth in SEQ ID NOs: 50, 52, 53, respectively, and wherein the VH, when paired with a light chain variable region (VL) comprising the amino acid sequence set forth in SEQ ID NO: 48 binds to CD79b;
(18) an immunoglobulin heavy chain or a fragment thereof comprising a VH comprising complementarity determining regions (CDRs) 1, 2, and 3 comprising the amino acid sequences set forth in SEQ ID NOs: 60, 62, 63, respectively, and wherein the VH, when paired with a light chain variable region (VL) comprising the amino acid sequence set forth in SEQ ID NO: 58 binds to CD79b;
(19) an immunoglobulin heavy chain or a fragment thereof comprising a VH comprising complementarity determining regions (CDRs) 1, 2, and 3 comprising the amino acid sequences set forth in SEQ ID NOs: 39, 41, 43, respectively, and wherein the VH, when paired with a light chain variable region (VL) comprising the amino acid sequence set forth in SEQ ID NO: 72 binds to CD79b
(20) an immunoglobulin light chain or a fragment thereof comprising a VL comprising CDRs 1, 2, and 3 comprising the amino acid sequences set forth in SEQ ID NOs: 44-46, respectively, and wherein the VL, when paired with a VH comprising the amino acid sequence set forth in SEQ ID NO: 71 binds to CD79b
(21) an immunoglobulin heavy chain or a fragment thereof comprising a VH comprising complementarity determining regions (CDRs) 1, 2, and 3 comprising the amino acid sequences set forth in SEO ID NOs: 40, 42, 43, respectively, and wherein the VH, when paired with a light chain variable region (VL) comprising the amino acid sequence set forth in SEQ ID NO: 72 binds to CD79b;
(22) an immunoglobulin heavy chain or a fragment thereof comprising a VH comprising complementarity determining regions (CDRs) 1, 2, and 3 comprising the amino acid sequences set forth in SEQ ID NOs: 19, 21, 23, respectively, and wherein the VH, when paired with a light chain variable region (VL) comprising the amino acid sequence set forth in SEQ ID NO: 70 binds to CD79b;
(23) an immunoglobulin light chain or a fragment thereof comprising a VL comprising complementarity determining regions (CDRs) 1, 2, and 3 comprising the amino acid sequences set forth in SEQ ID NOs: 24-26, respectively, and wherein the VL, when paired with a VH comprising the amino acid sequence set forth in SEQ ID NO: 69 binds to CD79b;
(24) an immunoglobulin heavy chain or a fragment thereof comprising a VH comprising complementarity determining regions (CDRs) 1, 2, and 3 comprising the amino acid sequences set forth in SEQ ID NOs: 20, 22, 23, respectively, and wherein the VH, when paired with a light chain variable region (VL) comprising the amino acid sequence set forth in SEQ ID NO: 70 binds to CD79b;
(25) an immunoglobulin heavy chain or a fragment thereof comprising a VH comprising complementarity determining regions (CDRs) 1, 2, and 3 comprising the amino acid sequences set forth in SEQ ID NOs: 49, 51, 53, respectively, and wherein the VH, when paired with a light chain variable region (VL) comprising the amino acid sequence set forth in SEQ ID NO: 73 binds to CD79b;
(26) an immunoglobulin light chain or a fragment thereof comprising a VL comprising CDRs 1, 2, and 3 comprising the amino acid sequences set forth in SEQ ID NOs: 54-56, respectively, and wherein the VL, when paired with a VH comprising the amino acid sequence set forth in SEQ ID NO: 74 binds to CD79b;
(27) an immunoglobulin heavy chain or a fragment thereof comprising a VH comprising complementarity determining regions (CDRs) 1, 2, and 3 comprising the amino acid sequences set forth in SEO ID NOs: 50, 52, 53, respectively, and wherein the VH, when paired with a light chain variable region (VL) comprising the amino acid sequence set forth in SEQ ID NO: 73 binds to CD79b
(28) an immunoglobulin heavy chain or a fragment thereof comprising a VH comprising complementarity determining regions (CDRs) 1, 2, and 3 comprising the amino acid sequences set forth in SEQ ID NOs: 59, 61, 63, respectively, and wherein the VH, when paired with a light chain variable region (VL) comprising the amino acid sequence set forth in SEQ ID NO: 76 binds to CD79b;
(29) an immunoglobulin light chain or a fragment thereof comprising a VL comprising CDRs 1, 2, and 3 comprising the amino acid sequences set forth in SEQ ID NOs: 64-66, respectively, and wherein the VL, when paired with a VH comprising the amino acid sequence set forth in SEQ ID NO: 75 binds to CD79b;
(30) an immunoglobulin heavy chain or a fragment thereof comprising a VH comprising complementarity determining regions (CDRs) 1, 2, and 3 comprising the amino acid sequences set forth in SEQ ID NOs: 60, 62, 63, respectively, and wherein the VH, when paired with a light chain variable region (VL) comprising the amino acid sequence set forth in SEQ ID NO: 76 binds to CD79b.

18.-39. (canceled)

40. A vector comprising one or more of the nucleic acids of claim 17.

41.-44. (canceled)

45. A cell comprising the nucleic acid of claim 17.

46. (canceled)

47. (canceled)

48. A method of producing an antibody or an antigen-binding fragment thereof, the method comprising
(a) culturing the cell of claim 45 under conditions sufficient for the cell to produce the antibody or the antigen-binding fragment; and
(b) collecting the antibody or the antigen-binding fragment produced by the cell.

49. The antibody or antigen-binding fragment thereof of claim 1, comprising
a heavy chain variable region (VH) comprising an amino acid sequence that is at least 90% identical to a selected VH sequence, and a light chain variable region (VL) comprising an amino acid sequence that is at least 90% identical to a selected VL sequence, wherein the selected VH sequence and the selected VL sequence are one of the following:
(1) the selected VH sequence is SEQ ID NO: 37 or 71, and the selected VL sequence is SEQ ID NO: 38 or 72;
(2) the selected VH sequence is SEQ ID NO: 7, and the selected VL sequence is SEQ ID NO: 8;
(3) the selected VH sequence is SEQ ID NO: 17 or 69, and the selected VL sequence is SEQ ID NO: 18 or 70;
(4) the selected VH sequence is SEQ ID NO: 27, and the selected VL sequence is SEQ ID NO: 28;
(5) the selected VH sequence is SEQ ID NO: 47 or 73, and the selected VL sequence is SEQ ID NO: 48 or 74; and
(6) the selected VH sequence is SEQ ID NO: 57 or 75, and the selected VL sequence is SEQ ID NO: 58 or 76.

50.-59. (canceled)

60. An antibody or antigen-binding fragment thereof that binds to CD79b comprising
a heavy chain variable region (VH) comprising VH CDR1, VH CDR2, and VH CDR3 that are identical to VH CDR1, VH CDR2, and VH CDR3 of a selected VH sequence; and a light chain variable region (VL) comprising VL CDR1, VL CDR2, and VL CDR3 that are identical to VL CDR1, VL CDR2, and VL CDR3 of a selected VL sequence, wherein the selected VH sequence and the selected VL sequence are one of the following:
(1) the selected VH sequence is SEQ ID NO: 37 or 71, and the selected VL sequence is SEQ ID NO: 38 or 72;
(2) the selected VH sequence is SEQ ID NO: 7, and the selected VL sequence is SEQ ID NO: 8;
(3) the selected VH sequence is SEQ ID NO: 17 or 69, and the selected VL sequence is SEQ ID NO: 18 or 70;
(4) the selected VH sequence is SEQ ID NO: 27, and the selected VL sequence is SEQ ID NO: 28;
(5) the selected VH sequence is SEQ ID NO: 47 or 73, and the selected VL sequence is SEQ ID NO: 48 or 74; and
(6) the selected VH sequence is SEQ ID NO: 57 or 75, and the selected VL sequence is SEQ ID NO: 58 or 76.

61.-63. (canceled)

64. An antibody or antigen-binding fragment thereof that cross-competes with the antibody or antigen-binding fragment thereof of claim 1.

65. The antibody or antigen-binding fragment thereof of claim 1, wherein the antibody or antigen-binding fragment thereof comprises a fragment crystallizable region (Fc region).

66. A chimeric antigen receptor (CAR) comprising the antibody or antigen-binding fragment thereof of claim 1.

67. An antibody-drug conjugate comprising the antibody or antigen-binding fragment thereof of any one of claim 1 covalently bound to a therapeutic agent.

68. The antibody drug conjugate of claim 67, wherein the therapeutic agent is a cytotoxic or cytostatic agent.

69. A method of treating a subject having cancer, the method comprising administering a therapeutically effective amount of a composition comprising the antibody or antigen-binding fragment thereof of claim 1, to the subject.

70. The method of claim 69, wherein the cancer is lymphoma, leukemia, breast cancer, stomach cancer, pancreatic cancer, prostate cancer, cervical cancer, endometrial cancer, ovarian cancer or urothelial cancer.

71. The method of claim 69, wherein the cancer is Non-Hodgkin lymphoma (NHL), Diffuse large B-cell lymphoma (DLBCL), B acute lymphoblastic leukemia (B-ALL), chronic lymphocytic leukemia (CLL), B-cell prolymphocytic leukemia (PLL), splenic lymphoma with villous lymphocytes (SLVL), hairy cell leukemia (HCL), follicular lymphoma (FL) or mantle-cell (MCL) lymphoma.

72.-75. (canceled)

76. A method of treating a subject having autoimmune disease, the method comprising administering a therapeutically effective amount of a composition comprising the antibody or antigen-binding fragment thereof of claim 1 to the subject.

77. (canceled)

78. (canceled)

79. A pharmaceutical composition comprising the antibody or antigen-binding fragment thereof of claim 1, and a pharmaceutically acceptable carrier.

80. (canceled)
